SEQUENCE LISTING (2 sequences)

SEQ ID NO: 1 - protein, 190 aa, synthetic construct
PPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEY

SEQ ID NO: 2 - protein, 267 aa, synthetic construct
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

(19) United States
(12) Patent Application Publication
(10) Pub. No.: US 2025/0230208 A1
(43) Pub. Date: Jul. 17, 2025

(54) METHODS FOR TREATING EYE DISEASES USING LIPID BINDING PROTEIN-BASED COMPLEXES

(71) Applicant: ABIONYX PHARMA SA, Balma (FR)

(72) Inventors: Cyrille TUPIN, Castelmaurou (FR); Jérôme Martinez, Paris (FR); Frédéric Lallemand, Rambouillet (FR)

(73) Assignee: ABIONYX PHARMA SA, Balma (FR)

(21) Appl. No.: 18/854,155

(22) PCT Filed: Apr. 6, 2023
(86) PCT No.: PCT/IB2023/000181

§ 371 Date: Oct. 4, 2024

Related U.S. Application Data
(60) Provisional application No. 63/328,088, filed on Apr. 6, 2022.

(51) Int. Cl. C07K 14/47; A61K 38/00; A61P 27/02

(52) U.S. Cl. CPC C07K 14/4717; A61P 27/02; A61K 38/00

(57) ABSTRACT

Disclosed herein are methods for treating eye diseases, for example eye diseases associated with lipid accumulation, with lipid binding protein-based complexes such as CER-001; lipid binding protein-based complexes, compositions comprising a lipid binding protein-based complex as a carrier for one or more ophthalmic drugs, and uses thereof.

### 1. CROSS-REFERENCE TO RELATED APPLICATIONS

This application claims priority to U.S. provisional application No. 63/328,088, filed Apr. 6, 2022, the contents of which are incorporated herein in their entireties by reference thereto.

### 2. SEQUENCE LISTING

The instant application contains a Sequence Listing which has been submitted electronically and is hereby incorporated by reference in its entirety. Said electronic copy, created on Mar. 28, 2023, is an XML file named CRN-041B-WO_ST26 and is 3,216 bytes in size.

### 3. BACKGROUND

### 3.1. Eye Diseases

The vertebrate eye is a complex sensory organ consisting of multiple, distinct tissues, each having its own unique biochemical composition, structure, and physiological function. Key among these are the retina, lens, and cornea, working in concert to bring photons of light into the eye, focus them correctly on the retina, and convert their energy into electrochemical signals that are conveyed to the brain where, ultimately, they are processed into a coherent visual image. Defects in any or all of these tissues, whether inborn or acquired, whether through a disease process or by traumatic injury, can compromise vision and, eventually, may result in complete and irreversible blindness. Lipids and lipid-soluble compounds are essential constituents of the cells and tissues that comprise the eye, and defects in their synthesis, intracellular and extracellular transport, and turnover underlie a variety of significant, common, and often severely debilitating eye diseases.

Diseases of the eye can have various causes, for example genetics, infection and aging. Some eye diseases are associated with lipid accumulation in the eye or near the eye, for example, fish-eye disease, dry eye diseases, for example associated with Meibomian gland dysfunction or lacrimal gland dysfunction, blepharitis, uveitis, diseases of the cornea such as lipid keratopathy, dry macular degeneration (dry AMD), Stargardt disease and Leber's idiopathic stellate neuroretinitis.

### 3.2. Lecithin Cholesterol Acyl Transferase

Lecithin cholesterol acyl transferase (LCAT) is an enzyme produced by the liver and is the key enzyme in the reverse cholesterol transport (RCT) pathway. The RCT pathway functions to eliminate cholesterol from most extrahepatic tissues and is crucial to maintaining the structure and function of most cells in the body. ROT consists mainly of three steps: (a) cholesterol efflux, i.e., the initial removal of cholesterol from various pools of peripheral cells; (b) cholesterol esterification by the action of lecithin: cholesterol acyltransferase (LCAT), preventing a re-entry of effluxed cholesterol into cells; and (c) uptake of high density lipoprotein (HDL)-cholesterol and cholesteryl esters to liver cells for hydrolysis, then recycling, storage, excretion in bile or catabolismto bile acids.

LCAT circulates in plasma associated with the HDL fraction. LCAT converts cell-derived cholesterol to cholesteryl esters, which are sequestered in HDL destined for removal (see Jonas 2000, Biochim. Biophys. Acta 1529 (1-3): 245-56). Cholesteryl ester transfer protein CETP) and phospholipid transfer protein (PLTP) contribute to further remodeling of the circulating HDL population. CETP moves cholesteryl esters made by LCAT to other lipoproteins, particularly ApoB-comprising lipoproteins, such as very low density lipoprotein (VLDL) and low density lipoprotein (LDL). PLTP supplies lecithin to HDL. HDL triglycerides are catabolized by the extracellular hepatic triglyceride lipase, and lipoprotein cholesterol is removed by the liver via several mechanisms.

A deficiency of LCAT causes accumulation of unesterified cholesterol in certain body tissues. Cholesterol effluxes from cells as free cholesterol and is transported in HDL as esterified cholesterol. LCAT is the enzyme that esterifies the free cholesterol on HDL to cholesterol ester and allows the maturation of HDL. LCAT deficiency does not allow for HDL maturation resulting in its rapid catabolism of circulating apoA-1 and apoA-2. The remaining form of HDL resembles nascent HDL. Subjects with LCAT deficiency (both full and partial) have low HDL cholesterol.

Familial LCAT deficiency is a rare genetic disorder in which sufferers lack LCAT activity and are of risk of progressive chronic kidney disease and in some cases renal failure. Fish eye disease is a partial LCAT deficiency in which LCAT cannot esterify, or make the acid into an alkyl, cholesterol in HDL particles. However, LCAT remains active on the cholesterol particles in VLDL and LDL.

### 3.3. Fish-Eye Disease

Fish-eye disease, also called partial LCAT deficiency, is a disorder that causes the clear front surface of the eyes (the corneas) to gradually become cloudy. The cloudiness, which generally first appears in adolescence or early adulthood, consists of small grayish dots of cholesterol (opacities) distributed across the corneas.

Fish-eye disease is characterized by abnormalities like visual impairment, plaques of fatty material, and dense opacification. Fish-eye disease is an autosomal recessive disorder caused by mutations of the LCAT gene located on chromosome 16q22.1.

LCAT gene mutations that cause fish-eye disease impair alpha-LCAT activity, reducing the enzyme's ability to attach cholesterol to HDL. Impairment of this mechanism for reducing cholesterol in the body leads to cholesterol-containing opacities in the corneas. It is not known why the cholesterol deposits affect only the corneas in this disorder. Mutations that affect both alpha-LCAT activity and beta-LCAT activity lead to a related disorder called complete LCAT deficiency, which involves corneal opacities in combination with features affecting other parts of the body.

Currently, there is no specific treatment to correct the LCAT deficiency so therapy is focused on symptom relief. In severe cases of fish-eye disease, corneal transplantation may be recommended.

New methods for treating subjects with eye diseases, for example eye diseases associated with lipid accumulation, are needed.

### 4. SUMMARY

In one aspect, the present disclosure provides methods for treating eye diseases, for example, eye diseases associated with lipid accumulation (e.g., in subjects having ocular lipid deposits), using lipid binding protein-based complexes, for example CER-001. Other lipid binding protein-based complexes that can be used in the methods of the disclosure include Apomers, Cargomers, and HDL based complexes or HDL mimetic-based complexes such as CSL-111, CSL-112, ETC-216 or delipidated HDL. In some eye diseases, lipids may accumulate in the eye or near the eye (e.g., lipids may accumulate in a subject's Meibomian gland or lacrimal gland). Exemplary eye diseases associated with lipid accumulation that can be treated by the methods of the disclosure include dry eye disease, such as dry eye disease associated with Meibomian gland dysfunction or lacrimal gland dysfunction, blepharitis, uveitis, diseases of the cornea such as lipid keratopathy, dry macular degeneration (dry AMD), Stargardt disease, Leber's idiopathic stellate neuroretinitis, and eye diseases associated with LCAT deficiency such as fish-eye disease. In some embodiments, the use of a lipid binding protein complex can reduce the severity of the eye disease. In some embodiments, the use of a lipid binding protein complex can slow the progression of the eye disease. Without being bound by theory, it is believed that a lipid binding protein complex can reduce ocular lipid deposits, for example by solubilizing the lipids accumulated in the ocular deposits, leading to their elimination.

In another aspect, the present disclosure provides methods of delivering ophthalmic drugs to the eye of a subject having an eye disease using a lipid binding protein-based complex (e.g., CER-001) as a drug carrier, thereby treating the eye disease. For example, the subject can be a subject suffering from an anterior ocular condition or a posterior ocular condition, for example uveitis, macular edema (e.g., diabetic macular edema), macular degeneration, retinal detachment, an ocular tumor, a fungal or viral infection, multifocal choroiditis, diabetic retinopathy, proliferative vitreoretinopathy (PVR), sympathetic ophthalmia, Vogt Koyanagi-Harada (VKH) syndrome, histoplasmosis, uveal diffusion, vascular occlusion, endophthalmitis, or glaucoma.

In another aspect, the present disclosure provides compositions comprising a lipid binding protein-based complex (e.g., CER-001) and one or more ophthalmic drugs complexed thereto.

In the methods described herein, the lipid binding protein-based complex (e.g., CER-001) can be administered systemically (e.g., by infusion). Alternatively, the lipid binding protein-based complex (e.g., CER-001) can be administered locally (e.g., by intraocular or topical administration). Intraocular administration can be by, for example, intraocular injection, for example intra-vitreal injection, sub-conjunctival injection, parabulbar injection, peribulbar injection or retro-bulbar injection. For topical administration, the lipid binding protein-based complex (e.g., CER-001) can be administered, for example, as an eye drop. Additional means of delivery can also be used, for example delivery via suprachoroidal injection, suprascleral injection, an implant (for example a disc, a sheet, a plug, a rod, or a pellet), iontophoresis, or electroporation.

In one aspect, the present disclosure provides dosing regimens for lipid binding protein-based complexes (e.g., CER-001) for treating subjects with eye diseases associated with lipid accumulation. The dosing regimens described herein can also be applied to deliver ophthalmic drugs to the eye using a lipid binding protein-based complex (e.g., CER-001) as a drug carrier.

The dosing regimens of the disclosure in some embodiments entail administering a lipid binding protein-based complex (e.g., CER-001) to a subject according to an initial “induction” regimen, followed by administering the lipid binding protein-based complex (e.g., CER-001) to the subject according to a “consolidation” regimen, followed by administering the lipid binding protein-based complex (e.g., CER-001) to the subject according to a “maintenance” regimen. Alternatively, dosing regimens can entail administering a lipid binding protein-based complex (e.g., CER-001) to the subject according to a “maintenance” regimen without a preceding “induction” regimen or “consolidation” regimen. As another alternative, dosing regimens can entail administering a lipid binding protein-based complex (e.g., CER-001) to the subject according to an “induction” regimen followed by a “maintenance” regimen without an intervening “consolidation” regimen.

The induction regimen typically comprises administering multiple doses of a lipid binding protein-based complex (e.g., CER-001) to the subject with a period of 1 day or greater between each dose. In some embodiments, the induction regimen comprises three or more doses of a lipid binding protein-based complex (e.g., CER-001). In some embodiments, the induction regimen comprises three doses a week of a lipid binding protein-based complex (e.g., CER-001). In some embodiments, the induction regimen comprises three doses a week of a lipid binding protein-based complex (e.g., CER-001) for a period of more than one week e.g., a period of two weeks or greater. In some embodiments the induction regimen comprises three doses a week of a lipid binding protein-based complexes (e.g., CER-001) for a period of three weeks.

The consolidation regimen typically comprises administering multiple doses of a lipid binding protein-based complex (e.g., CER-001) to the subject on a less frequent basis than during the induction regimen. The consolidation regimen typically comprises administering multiple doses of a lipid binding protein-based complex (e.g., CER-001) to the subject with a period of 1 day or greater between each dose e.g., 2 days or greater between each dose. In some embodiments, the consolidation regimen comprises two or more doses of a lipid binding protein-based complex (e.g., CER-001). In some embodiments, the consolidation regimen comprises two doses a week of a lipid binding protein-based complex (e.g., CER-001). In some embodiments, the consolidation regimen comprises two doses a week of a lipid binding protein-based complex (e.g., CER-001) for a period of more than one week e.g., a period of two weeks or greater. In some embodiments the consolidation regimen comprises two doses a week of a lipid binding protein-based complex (e.g., CER-001) for a period of three weeks.

The maintenance regimen typically comprises administering one or more doses of a lipid binding protein-based complex (e.g., CER-001) to the subject on a less frequent basis than during the consolidation regimen, for example a period of 5 days or greater, e.g., a period of one week, between doses. In certain embodiments, the multiple doses of a lipid binding protein-based complex (e.g., CER-001) are administered once every week during the maintenance regimen.

In certain aspects, the disclosure provides methods of treating a subject with lipid binding protein-based complexes (e.g., CER-001) using an induction regimen comprising administering three doses of the lipid binding protein-based complexes (e.g., CER-001) to the subject within one week for three weeks with at least 1 day between each dose followed by a consolidation regimen comprising administering two doses of the lipid binding protein-based complex (e.g., CER-001) to the subject within one week for three weeks with at least 2 days between each dose followed by a maintenance regimen comprising administering one dose of the lipid binding protein-based complex (e.g., CER-001) to the subject every week.

In certain aspects, the disclosure provides methods of treating a subject with a lipid binding protein-based complex (e.g., CER-001) in accordance with a dosage regimen described herein. In some embodiments, the lipid binding protein-based complex (e.g., CER-001) is diluted with saline before intravenous administration such as intravenous infusion using an infusion pump. In certain embodiments the dose of the lipid binding protein-based complex (e.g., CER-001) for infusion is based on subject weight, for example 10 mg/kg on a protein weight basis.

In certain aspects, the disclosure provides methods of treating a subject having an eye disease (e.g., associated with lipid accumulation) with a lipid binding protein-based complex (e.g., CER-001) according to a dosage regimen comprising:
3 doses per week for 3 weeks (induction regimen) followed by
2 doses per week for 3 weeks (consolidation regimen), followed by
1 dose per week until the end of treatment (maintenance regimen).

In certain aspects, an antihistamine (e.g., dexchlorpheniramine, hydroxyzine, diphenhydramine, cetirizine, fexofenadine, or loratadine) can be administered before administration of the lipid binding protein-based complex (e.g., CER-001), e.g., when the lipid binding protein-based complex is administered by IV infusion. The antihistamine can reduce the likelihood of allergic reactions.

The subject treated according to the dosing regimens of the disclosure can be any subject suffering from an eye disease associated with lipid accumulation, for example a subject having LCAT deficiency. The LCAT deficiency may be full LCAT deficiency or partial LCAT deficiency. In some embodiments, the subject treated according to the dosing regimens of the disclosure has fish-eye disease. Alternatively, subjects treated according to the dosing regimens of the disclosure can also be any subject in need of treatment with an ophthalmic drug, where the drug is delivered to the eye using a lipid binding protein-based complex (e.g., CER-001) as a drug carrier.

### 5. BRIEF DESCRIPTION OF THE FIGURES

FIG. 1A-1D: show the ability of CER-001 to act as a drug carrier for ophthalmic drugs azithromycin (FIG. 1A), spironolactone (FIG. 1B), dexamethasone palmitate (FIG. 1C) and cyclosporine (FIG. 1D).

FIGS. 2A-2C: show tolerance scores from rabbits administered CER-001, with or without complexed dexamethasone palmitate (Example 4). FIG. 2A: plot of tolerance at 6 and 24 hours; FIG. 2B: tolerance at 6 hours; FIG. 2C: tolerance at 24 hours.

FIGS. 3A-3B: show cell infiltration (FIG. 3A) and protein (FIG. 3B) in aqueous humor of rabbits administered CER-001, with or without complexed dexamethasone palmitate (Example 4).

FIG. 4: shows haze in rabbit eyes administered with CER-001 and soft corticoid (CER-S Cort), at 0 hours, 6 hours, and 24 hours after administration.

### 6. DETAILED DESCRIPTION

In some aspects, the disclosure provides methods for treating eye diseases (e.g., eye diseases associated with lipid accumulation) using a lipid binding protein-based complex (e.g., CER-001). The methods of the disclosure can reduce the severity of a subject's eye disease. In some embodiments, the lipid binding protein-based complex is an Apomer, a Cargomer, a HDL based complex, or a HDL mimetic based complex. In some embodiments, the lipid binding protein-based complex can be used as a drug carrier to deliver one or more ophthalmic drugs to the eye, e.g., one or more ophthalmic drugs which are hydrophobic and/or poorly water soluble or water insoluble.

In some embodiments, the lipid binding protein-based complex (e.g., CER-001) (e.g., when used as a drug carrier or not used as a drug carrier) does not comprise and is not administered with a cell-penetrating peptide (CPP) (e.g., a CPP as described in WO 2019/018350), chemical penetration enhancer (CPE) (e.g., a CPE as described in WO 2019/018350) or a cytophilic peptide (e.g., a cytophilic peptide as described in EP 3 238 746 A1). The contents of WO 2019/018350 and EP 3 238 746 A1 are incorporated herein by reference in their entireties.

In additional aspects, the disclosure provides lipid binding protein-based complexes such as CER-001 for use as a carrier for one or more ophthalmic drugs. Accordingly, in some aspects, the disclosure provides compositions comprising a lipid binding protein-based complex (e.g., CER-001) with one or more ophthalmic drugs (e.g., as described in Section 6.1.9) complexed thereto. Such compositions can be used in the methods of the disclosure.

Exemplary features of lipid binding protein-based complexes that can be used in the methods and compositions of the disclosure are described in Section 6.1. Exemplary subject populations who can be treated by the methods of the disclosure and with the compositions of the disclosure are described in Section 6.2.

Lipid binding protein-based complexes can be administered peripherally or locally. In some embodiments, a lipid binding protein-based complex is administered peripherally, for example by infusion. In other embodiment, a lipid binding protein-based complex is administered locally (e.g., by intraocular or topical administration).

In some embodiments, methods of the disclosure comprise administering a lipid binding protein-based complex (e.g., CER-001) to a subject in three phases. First, the lipid binding protein-based complex (e.g., CER-001) is administered in an initial, intense “induction” regimen. The induction regimen is followed by a less intense “consolidation” regimen. The consolidation regimen is followed by a “maintenance” regimen. In other methods of the disclosure, a lipid binding protein-based complex (e.g., CER-001) is administered in two phases (e.g., an induction regimen followed by a maintenance regiment) or a single phase (e.g., a maintenance regimen). Induction regimens that can be used in the methods of the disclosure are described in Section 6.3, consolidation regimens that can be used in the methods of the disclosure are described in Section 6.4 and maintenance regimens that can be used in the methods of the disclosure are described in Section 6.5. The dosing regimens of the disclosure comprise administering a lipid binding protein-based complex (e.g., CER-001) as monotherapy or as part of a combination therapy with one or more medications. Combination therapies are described in Section 6.6.

### 6.1. Lipid Binding Protein-Based Complexes

### 6.1.1. HDL and HDL Mimetic-Based Complexes

In one aspect, the lipid binding protein-based complexes comprise HDL or HDL mimetic-based complexes. For example, complexes can comprise a lipoprotein complex as described in U.S. Pat. No. 8,206,750, PCT publication WO 2012/109162, PCT publication WO 2015/173633 A2 (e.g., CER-001), PCT publication WO 2004/073684, or US 2004/0229794 A1, the contents of each of which are incorporated herein by reference in their entireties. The terms “lipoproteins” and “apolipoproteins” are used interchangeably herein, and unless required otherwise by context, the term “lipoprotein” encompasses lipoprotein mimetics. The terms “lipid binding protein” and “lipid binding polypeptide” are also used interchangeably herein, and unless required otherwise by context, the terms do not connote an amino acid sequence of particular length.

Lipoprotein complexes can comprise a protein fraction (e.g., an apolipoprotein fraction) and a lipid fraction (e.g., a phospholipid fraction). The protein fraction includes one or more lipid-binding protein molecules, such as apolipoproteins, peptides, or apolipoprotein peptide analogs or mimetics, for example one or more lipid binding protein molecules described in Section 6.1.4. In some embodiments, the lipid-binding protein molecule(s) comprise apolipoprotein molecule(s) (e.g., ApoA-I molecule(s)), but not apolipoprotein mimetic molecule(s).

The lipid fraction typically includes one or more phospholipids which can be neutral, negatively charged, positively charged, or a combination thereof. Exemplary phospholipids and other amphipathic molecules which can be included in the lipid fraction are described in Section 6.1.6.

In certain embodiments, the lipid fraction contains at least one neutral phospholipid (e.g., a sphingomyelin (SM)) and, optionally, one or more negatively charged phospholipids. In lipoprotein complexes that include both neutral and negatively charged phospholipids, the neutral and negatively charged phospholipids can have fatty acid chains with the same or different number of carbons and the same or different degree of saturation. In some instances, the neutral and negatively charged phospholipids will have the same acyl tail, for example a C16:0, or palmitoyl, acyl chain. In specific embodiments, particularly those in which egg SM is used as the neutral lipid, the weight ratio of the apolipoprotein fraction:lipid fraction ranges from about 1:2.7 to about 1:3 (e.g., 1:2.7).

Any phospholipid that bears at least a partial negative charge at physiological pH can be used as the negatively charged phospholipid. Non-limiting examples include negatively charged forms, e.g., salts, of phosphatidylinositol, a phosphatidylserine, a phosphatidylglycerol and a phosphatidic acid. In a specific embodiment, the negatively charged phospholipid is 1,2-dipalmitoyl-sn-glycero-3-[phospho-rac-(1-glycerol)], or DPPG, a phosphatidylglycerol. Preferred salts include potassium and sodium salts.

In some embodiments, a lipoprotein complex used in the compositions and methods of the disclosure is a lipoprotein complex as described in U.S. Pat. No. 8,206,750 or WO 2012/109162 (and its U.S. counterpart, US 2012/0232005), the contents of each of which are incorporated herein in its entirety by reference. In particular embodiments, the protein component of the lipoprotein complex is as described in Section 6.1 and preferably in Section 6.1.1 of WO 2012/109162 (and US 2012/0232005), the lipid component is as described in Section 6.2 of WO 2012/109162 (and US 2012/0232005), which can optionally be complexed together in the amounts described in Section 6.3 of WO 2012/109162 (and US 2012/0232005). The contents of each of these sections are incorporated by reference herein. In certain aspects, a lipoprotein complex of the disclosure is in a population of complexes that is at least 85%, at least 90%, at least 95%, at least 97%, or at least 99% homogeneous, as described in Section 6.4 of WO 2012/109162 (and US 2012/0232005), the contents of which are incorporated by reference herein.

In a specific embodiment, a lipoprotein complex that can be used in the compositions and methods of the disclosure comprises 2-4 ApoA-I equivalents, 2 molecules of charged phospholipid, 50-80 molecules of lecithin and 20-50 molecules of SM.

In another specific embodiment, a lipoprotein complex that can be used in the compositions and methods of the disclosure comprises 2-4 ApoA-I equivalents, 2 molecules of charged phospholipid, 50 molecules of lecithin and 50 molecules of SM.

In yet another specific embodiment, a lipoprotein complex that can be used in the compositions and methods of the disclosure comprises 2-4 ApoA-I equivalents, 2 molecules of charged phospholipid, 80 molecules of lecithin and 20 molecules of SM.

In yet another specific embodiment, a lipoprotein complex that can be used in the compositions and methods of the disclosure comprises 2-4 ApoA-I equivalents, 2 molecules of charged phospholipid, 70 molecules of lecithin and 30 molecules of SM.

In yet another specific embodiment, a lipoprotein complex that can be used in the compositions and methods of the disclosure comprises 2-4 ApoA-I equivalents, 2 molecules of charged phospholipid, 60 molecules of lecithin and 40 molecules of SM.

In a specific embodiment, a lipoprotein complex that can be used in the compositions and methods of the disclosure consists essentially of 2-4 ApoA-I equivalents, 2 molecules of charged phospholipid, 50-80 molecules of lecithin and 20-50 molecules of SM.

In another specific embodiment, a lipoprotein complex that can be used in the compositions and methods of the disclosure consists essentially of 2-4 ApoA-I equivalents, 2 molecules of charged phospholipid, 50 molecules of lecithin and 50 molecules of SM.

In yet another specific embodiment, a lipoprotein complex that can be used in the compositions and methods of the disclosure consists essentially of 2-4 ApoA-I equivalents, 2 molecules of charged phospholipid, 80 molecules of lecithin and 20 molecules of SM.

In yet another specific embodiment, a lipoprotein complex that can be used in the compositions and methods of the disclosure consists essentially of 2-4 ApoA-I equivalents, 2 molecules of charged phospholipid, 70 molecules of lecithin and 30 molecules of SM.

In yet another specific embodiment, a lipoprotein complex that can be used in the compositions and methods of the disclosure consists essentially of 2-4 ApoA-I equivalents, 2 molecules of charged phospholipid, 60 molecules of lecithin and 40 molecules of SM.

In a specific embodiment, a lipoprotein complex that can be used in the compositions and methods of the disclosure comprises a lipid component that comprises about 90 to 99.8 wt % SM and about 0.2 to 10 wt % negatively charged phospholipid, for example, about 0.2-1 wt %, 0.2-2 wt %, 0.2-3 wt %, 0.2-4 wt %, 0.2-5 wt %, 0.2-6 wt %, 0.2-7 wt %, 0.2-8 wt %, 0.2-9 wt %, or 0.2-10 wt % total negatively charged phospholipid(s). In another specific embodiment, a lipoprotein complex that can be used in the methods of the disclosure comprises about 90 to 99.8 wt % lecithin and about 0.2 to 10 wt % negatively charged phospholipid, for example, about 0.2-1 wt %, 0.2-2 wt %, 0.2-3 wt %, 0.2-4 wt %, 0.2-5 wt %, 0.2-6 wt %, 0.2-7 wt %, 0.2-8 wt %, 0.2-9 wt % or 0.2-10 wt % total negatively charged phospholipid(s).

In a specific embodiment, a lipoprotein complex that can be used in the compositions and methods of the disclosure comprises a lipid component that consists essentially of about 90 to 99.8 wt % SM and about 0.2 to 10 wt % negatively charged phospholipid, for example, about 0.2-1 wt %, 0.2-2 wt %, 0.2-3 wt %, 0.2-4 wt %, 0.2-5 wt %, 0.2-6 wt %, 0.2-7 wt %, 0.2-8 wt %, 0.2-9 wt %, or 0.2-10 wt % total negatively charged phospholipid(s). In another specific embodiment, a lipoprotein complex that can be used in the methods of the disclosure consists essentially of about 90 to 99.8 wt % lecithin and about 0.2 to 10 wt % negatively charged phospholipid, for example, about 0.2-1 wt %, 0.2-2 wt %, 0.2-3 wt %, 0.2-4 wt %, 0.2-5 wt %, 0.2-6 wt %, 0.2-7 wt %, 0.2-8 wt %, 0.2-9 wt % or 0.2-10 wt % total negatively charged phospholipid(s).

In still another specific embodiment, a lipoprotein complex that can be used in the compositions and methods of the disclosure comprises a lipid fraction that comprises about 9.8 to 90 wt % SM, about 9.8 to 90 wt % lecithin and about 0.2-10 wt % negatively charged phospholipid, for example, from about 0.2-1 wt %, 0.2-2 wt %, 0.2-3 wt %, 0.2-4 wt %, 0.2-5 wt %, 0.2-6 wt %, 0.2-7 wt %, 0.2-8 wt %, 0.2-9 wt %, to 0.2-10 wt % total negatively charged phospholipid(s).

In still another specific embodiment, a lipoprotein complex that can be used in the compositions and methods of the disclosure comprises a lipid fraction that consists essentially of about 9.8 to 90 wt % SM, about 9.8 to 90 wt % lecithin and about 0.2-10 wt % negatively charged phospholipid, for example, from about 0.2-1 wt %, 0.2-2 wt %, 0.2-3 wt %, 0.2-4 wt %, 0.2-5 wt %, 0.2-6 wt %, 0.2-7 wt %, 0.2-8 wt %, 0.2-9 wt %, to 0.2-10 wt % total negatively charged phospholipid(s).

In another specific embodiment, a lipoprotein complex that can be used in the compositions and methods of the disclosure comprises an ApoA-I apolipoprotein and a lipid fraction, wherein the lipid fraction comprises sphingomyelin and about 3 wt % of a negatively charged phospholipid, wherein the molar ratio of the lipid fraction to the ApoA-I apolipoprotein is about 2:1 to 200:1, and wherein said complex is a small or large discoidal particle containing 2-4 ApoA-I equivalents.

In another specific embodiment, a lipoprotein complex that can be used in the compositions and methods of the disclosure comprises an ApoA-I apolipoprotein and a lipid fraction, wherein the lipid fraction consists essentially of sphingomyelin and about 3 wt % of a negatively charged phospholipid, wherein the molar ratio of the lipid fraction to the ApoA-I apolipoprotein is about 2:1 to 200:1, and wherein said complex is a small or large discoidal particle containing 2-4 ApoA-I equivalents.

HDL-based or HDL mimetic-based complexes can include a single type of lipid-binding protein, or mixtures of two or more different lipid-binding proteins, which may be derived from the same or different species. Although not required, the complexes will preferably comprise lipid-binding proteins that are derived from, or correspond in amino acid sequence to, the animal species being treated, in order to avoid inducing an immune response to the therapy.

Thus, for treatment of human patients, lipid-binding proteins of human origin are preferably used. The use of peptide mimetic apolipoproteins may also reduce or avoid an immune response.

In some embodiments, the lipid component includes two types of phospholipids: a sphingomyelin (SM) and a negatively charged phospholipid. Exemplary SMs and negatively charged lipids are described in Section 6.1.6.1.

Lipid components including SM can optionally include small quantities of additional lipids. Virtually any type of lipids may be used, including, but not limited to, lysophospholipids, galactocerebroside, gangliosides, cerebrosides, glycerides, triglycerides, and cholesterol and its derivatives.

When included, such optional lipids will typically comprise less than about 15 wt % of the lipid fraction, although in some instances more optional lipids could be included. In some embodiments, the optional lipids comprise less than about 10 wt %, less than about 5 wt %, or less than about 2 wt %. In some embodiments, the lipid fraction does not include optional lipids.

In a specific embodiment, the phospholipid fraction contains egg SM or palmitoyl SM or phytosphingomyelin and DPPG in a weight ratio (SM: negatively charged phospholipid) ranging from 90:10 to 99:1, more preferably ranging from 95:5 to 98:2. In one embodiment, the weight ratio is 97:3.

The molar ratio of the lipid component to the protein component of complexes of the disclosure can vary, and will depend upon, among other factors, the identity(ies) of the apolipoprotein comprising the protein component, the identities and quantities of the lipids comprising the lipid component, and the desired size of the complex. Because the biological activity of apolipoproteins such as ApoA-I are thought to be mediated by the amphipathic helices comprising the apolipoprotein, it is convenient to express the apolipoprotein fraction of the lipid:apolipoprotein molar ratio using ApoA-I protein equivalents. It is generally accepted that ApoA-I contains 6-10 amphipathic helices, depending upon the method used to calculate the helices. Other apolipoproteins can be expressed in terms of ApoA-I equivalents based upon the number of amphipathic helices they contain. For example, ApoA-IM, which typically exists as a disulfide-bridged dimer, can be expressed as 2 ApoA-I equivalents, because each molecule of ApoA-IM contains twice as many amphipathic helices as a molecule of ApoA-I. Conversely, a peptide apolipoprotein that contains a single amphipathic helix can be expressed as a 1/10-1/6 ApoA-I equivalent, because each molecule contains 1/10-1/6 as many amphipathic helices as a molecule of ApoA-I. In general, the lipid:ApoA-I equivalent molar ratio of the lipoprotein complexes (defined herein as “Ri”) will range from about 105:1 to 110:1. In some embodiments, the Ri is about 108:1. Ratios in weight can be obtained using a MW of approximately 650-800 for phospholipids.

In some embodiments, the molar ratio of lipid:ApoA-I equivalents (“RSM”) ranges from about 80:1 to about 110:1, e.g., about 80:1 to about 100:1. In a specific example, the RSM for complexes can be about 82:1.

In some embodiments, lipoprotein complexes used in the methods of the disclosure are negatively charged complexes which comprise a protein fraction which is preferably mature, full-length ApoA-I, and a lipid fraction comprising a neutral phospholipid, sphingomyelin (SM), and negatively charged phospholipid.

In a specific embodiment, the lipid component contains SM (e.g., egg SM, palmitoyl SM, phytoSM, or a combination thereof) and negatively charged phospholipid (e.g., DPPG) in a weight ratio (SM:negatively charged phospholipid) ranging from 90:10 to 99:1, more preferably ranging from 95:5 to 98:2, e.g., 97:3.

In specific embodiments, the ratio of the protein component to lipid component can range from about 1:2.7 to about 1:3, with 1:2.7 being preferred. This corresponds to molar ratios of ApoA-I protein to lipid ranging from approximately 1:90 to 1:140. In some embodiments, the molar ratio of protein to lipid in the complex is about 1:90 to about 1:120, about 1:100 to about 1:140, or about 1:95 to about 1:125.

In particular embodiments, the complex comprises CER-001, CSL-111, CSL-112, CER-522 or ETC-216. In a preferred embodiment, the complex is CER-001.

CER-001 as used in the literature and in the Examples below refers to a complex described in Example 4 of WO 2012/109162. WO 2012/109162 refers to CER-001 as a complex having a 1:2.7 lipoprotein weight:total phospholipid weight ratio with a SM:DPPG weight:weight ratio of 97:3. Example 4 of WO 2012/109162 also describes a method of its manufacture.

When used in the context of a CER-001 dosing regimen or composition of the disclosure, CER-001 refers to a lipoprotein complex whose individual constituents can vary from CER-001 as described in Example 4 of WO 2012/109162 by up to 20%. In certain embodiments, the constituents of the lipoprotein complex vary from CER-001 as described in Example 4 of WO 2012/109162 by up to 10%. Preferably, the constituents of the lipoprotein complex are those described in Example 4 of WO 2012/109162 (plus/minus acceptable manufacturing tolerance variations). The SM in CER-001 can be natural or synthetic. In some embodiments, the SM is a natural SM, for example a natural SM described in WO 2012/109162, e.g., chicken egg SM. In some embodiments, the SM is a synthetic SM, for example a synthetic SM described in WO 2012/109162, e.g., synthetic palmitoylsphingomyelin, for example as described in WO 2012/109162. Methods for synthesizing palmitoylsphingomyelin are known in the art, for example as described in WO 2014/140787. The lipoprotein in CER-001, apolipoprotein A-I (ApoA-I), preferably has an amino acid sequence corresponding to amino acids 25 to 267 of SEQ ID NO:1 of WO 2012/109162 (said SEQ ID NO:1 of WO 2012/109162 disclosed herein as SEQ ID NO:2). ApoA-I can be purified by animal sources (and in particular from human sources) or produced recombinantly. In preferred embodiments, the ApoA-I in CER-001 is recombinant ApoA-I. CER-001 used in a dosing regimen of the disclosure is preferably highly homogeneous, for example at least 80%, at least 85%, at least 90%, at least 95%, at least 97%, at least 98%, or at least 99% homogeneous, as reflected by a single peak in gel permeation chromatography. See, e.g., Section 6.4 of WO 2012/109162.

CSL-111 is a reconstituted human ApoA-I purified from plasma complexed with soybean phosphatidylcholine (SBPC) (Tardif et al., 2007, JAMA 297:1675-1682).

CSL-112 is a formulation of ApoA-I purified from plasma and reconstituted to form HDL suitable for intravenous infusion (Diditchenko et al., 2013, DOI 10.1161/ATVBAHA.113.301981).

ETC-216 (also known as MDCO-216) is a lipid-depleted form of HDL containing recombinant ApoA-I_{Milano}. See Nicholls et al., 2011, Expert Opin Biol Ther. 11 (3): 387-94. doi: 10.1517/14712598.2011.557061.

In another embodiment, a complex that can be used in the methods of the disclosure is CER-522. CER-522 is a lipoprotein complex comprising a combination of three phospholipids and a 22 amino acid peptide, CT80522:

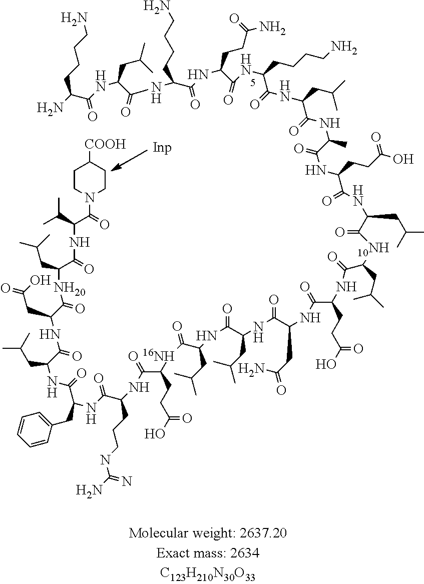

### CT80522

The phospholipid component of CER-522 consists of egg sphingomyelin, 1,2-dipalmitoyl-sn-glycero-3-phosphocholine (Dipalmitoylphosphatidylcholine, DPPC) and 1,2-dipalmitoyl-sn-glycero-3-[phospho-rac-(1-glycerol)] (Dipalmitoylphosphatidyl-glycerol, DPPG) in a 48.5:48.5:3 weight ratio. The ratio of peptide to total phospholipids in the CER-522 complex is 1:2.5 (w/w).

In some embodiments, the lipoprotein complex is delipidated HDL. Most HDL in plasma is cholesterol-rich. The lipids in HDL can be depleted, for example partially and/or selectively depleted, e.g., to reduce its cholesterol content. In some embodiments, the delipidated HDL can resemble small a, preβ-1, and other preβ forms of HDL. A process for selective depletion of HDL is described in Sacks et al., 2009, J Lipid Res. 50 (5): 894-907.

In certain embodiments, a lipoprotein complex comprises a bioactive agent delivery particle as described in US 2004/0229794.

A bioactive agent delivery particle can comprise a lipid binding polypeptide (e.g., an apolipoprotein as described previously in this Section or in Section 6.1.4), a lipid bilayer (e.g., comprising one or more phospholipids as described previously in this Section or in Section 6.1.6.1), and a bioactive agent (e.g., an anti-cancer agent), wherein the interior of the lipid bilayer comprises a hydrophobic region, and wherein the bioactive agent is associated with the hydrophobic region of the lipid bilayer. In some embodiments, a bioactive agent delivery particle as described in US 2004/0229794.

In some embodiments, a bioactive agent delivery particle does not comprise a hydrophilic core.

In some embodiments, a bioactive agent delivery particle is disc shaped (e.g., having a diameter from about 7 to about 29 nm).

Bioactive agent delivery particles include bilayer-forming lipids, for example phospholipids (e.g., as described previously in this Section or in Section 6.1.6.1). In some embodiments, a bioactive agent delivery particle includes both bilayer-forming and non-bilayer-forming lipids. In some embodiments, the lipid bilayer of a bioactive agent delivery particle includes phospholipids. In one embodiment, the phospholipids incorporated into a delivery particle include dimyristoylphosphatidylcholine (DMPC) and dimyristoylphosphatidylglycerol (DMPG). In one embodiment, the lipid bilayer includes DMPC and DMPG in a 7:3 molar ratio.

In some embodiments, the lipid binding polypeptide is an apolipoprotein (e.g., as described previously in this Section or in Section 6.1.4). The predominant interaction between lipid binding polypeptides, e.g., apolipoprotein molecules, and the lipid bilayer is generally a hydrophobic interaction between residues on a hydrophobic face of an amphipathic structure, e.g., an α-helix of the lipid binding polypeptide and fatty acyl chains of lipids on an exterior surface at the perimeter of the particle. Bioactive agent delivery particles may include exchangeable and/or non-exchangeable apolipoproteins. In one embodiment, the lipid binding polypeptide is ApoA-I.

In some embodiments, bioactive agent delivery particles include lipid binding polypeptide molecules, e.g., apolipoprotein molecules, that have been modified to increase stability of the particle. In one embodiment, the modification includes introduction of cysteine residues to form intramolecular and/or intermolecular disulfide bonds.

In another embodiment, bioactive agent delivery particles include a chimeric lipid binding polypeptide molecule, e.g., a chimeric apolipoprotein molecule, with one or more bound functional moieties, for example one or more targeting moieties and/or one or more moieties having a desired biological activity, e.g., antimicrobial activity, which may augment or work in synergy with the activity of a bioactive agent incorporated into the delivery particle.

### 6.1.2. Apomer Based Complexes

In one aspect, lipid binding protein-based complexes that can be used in the methods and compositions of the disclosure comprise Apomers. Features of Apomers that can be included in Apomer based complexes are described in WO/2019/030575, the contents of which are incorporated herein by reference in their entireties.

Apomers generally comprise an apolipoprotein in monomeric or multimeric form complexed with amphipathic molecules. Generally, Apomers comprise one or more apolipoprotein molecules, each complexed with one or more amphipathic molecules. In certain aspects, the amphipathic molecules together contribute a net charge of at least +1 or −1 per apolipoprotein molecule in an Apomer. Exemplary apolipoproteins that can be used in Apomers are described in Section 6.1.5.1. Exemplary amphipathic molecules are described in Section 6.1.6.

### 6.1.3. Cargomer Based Complexes

In one aspect, lipid binding protein-based complexes that can be used in the methods and compositions of the disclosure comprise Cargomers, which are lipid binding protein-based complexes having one or more cargo moieties. Features of Cargomers that can be included in Cargomer based complexes are described in WO/2019/030574, the contents of which are incorporated herein by reference in their entireties.

Cargomers generally comprise an apolipoprotein in monomeric or multimeric form (e.g., 2, 4, or 8 apolipoprotein molecules) and one or more cargo moieties. Cargo moieties can be amphipathic or non-amphipathic. Amphipathic cargo moieties can solubilize the apolipoprotein and prevent it from aggregating. Where the cargo moieties are not amphipathic or insufficient to solubilize the apolipoprotein molecule(s), the Cargomers can also comprise one or more additional amphipathic molecules to solubilize the apolipoprotein. Thus, reference to amphipathic molecules in the context of the Cargomers encompasses amphipathic molecules that are cargo moieties, amphipathic molecules that are not cargo moieties, or some combination thereof. Preferably, Cargomers are not discoidal, for example as determined using NMR spectroscopy.

Cargo moieties can include biologically active molecules (e.g., drugs, biologics, and/or immunogens) or other agents, for example agents used in diagnostics. As used herein, the terms “molecule” and “agent” also include complexes and conjugates (for example, antibody-drug conjugates). The terms “biologically active,” “diagnostically useful” and the like are not limited to substances with direct pharmacological or biological activity, and may include substances that become active following administration, for example due to metabolism of a prodrug or cleavage of a linker. According, the terms “biologically active” and “diagnostically useful” also includes substances that become biologically active or diagnostically useful after administration, through creation or metabolites or other cleavage products that exert a pharmacological or a biological effect and/or are detectable in a diagnostic test.

Amphipathic molecules in a Cargomer can solubilize the apolipoprotein and/or reduce or minimize apolipoprotein aggregation, and can also have other functions in the Cargomer. For example, amphipathic molecules can have therapeutic utility, and thus may be cargo moieties intended for delivery by the Cargomer upon administration to a subject. Additionally, as discussed in Section 6.1.6 below, amphipathic molecules can be used to anchor a non-amphipathic cargo moiety to the apolipoprotein in the Cargomer. Thus, in some embodiments, a cargo moiety and an amphipathic molecule in a Cargomer are the same. In other embodiments, an anchor moiety and an amphipathic molecule in a Cargomer are the same. In yet other embodiments, cargo moieties, anchor moieties and amphipathic molecules in a Cargomer are the same (for example, where an amphipathic molecule has therapeutic activity and also anchors another biologically active molecule to the apolipoprotein molecule(s)).

Anchor and/or linker moieties are particularly useful for a Cargomer having a cargo moiety that is not an amphipathic molecule.

In some embodiments, at least one of the cargo moieties, a majority of the cargo moieties, or all of the cargo moieties in a Cargomer of the disclosure are coupled to the Cargomer via anchors. In some embodiments, at least one of the cargo moieties in a Cargomer is coupled to the Cargomer via an anchor. In some embodiments, a majority of the cargo moieties in a Cargomer are coupled to the Cargomer via anchors. In some embodiments, all of the cargo moieties in a Cargomer are coupled to the Cargomer via anchors. Each anchor in a Cargomer can be the same or, alternatively, different types of anchors can be included in a single Cargomer (e.g., one type of cargo moiety can be coupled to the Cargomer via one type of anchor and a second type of cargo moiety can be coupled to the Cargomer via a second type of anchor).

In certain aspects, the amphipathic molecules, the cargo, and, if present, the anchors and/or linkers together contribute a net charge of at least +1 or −1 per apolipoprotein molecule in the Cargomer (e.g., +1, +2, +3, −1, −2, or −3). In some embodiments, the net charge is a negative charge. In other embodiments, the net charge is a positive charge. Unless required otherwise by context, charge is measured at physiological pH.

The molar ratio of apolipoprotein molecules to amphipathic molecules in a Cargomer can be but does not necessarily have to be in integers or reflect a one to one relationship between the apolipoprotein and amphipathic molecules. By way of example and not limitation, a Cargomer can have an apolipoprotein to amphipathic molecule molar ratio of 2:5, 8:7, 3:2, or 4:7.

In some embodiments, a Cargomer comprises apolipoprotein molecules complexed with amphipathic molecules in an apolipoprotein:amphipathic molecule molar ratio ranging from 8:1 to 1:15 (e.g., from 8:1 to 1:15, from 7:1 to 1:15, from 6:1 to 1:15, from 5:1 to 1:15, from 4:1 to 1:15, from 3:1 to 1:15, from 2:1 to 1:15, from 1:1 to 1:15, from 8:1 to 1:14, from 7:1 to 1:14, from 6:1 to 1:14, from 5:1 to 1:14, from 4:1 to 1:14, from 3:1 to 1:14, from 2:1 to 1:14, from 1:1 to 1:14, from 8:1 to 1:13, from 7:1 to 1:13, from 6:1 to 1:13, from 5:1 to 1:13, from 4:1 to 1:13, from 3:1 to 1:13, from 2:1 to 1:13, from 1:1 to 1:13, from 8:1 to 1:12, from 7:1 to 1:12, from 6:1 to 1:12, from 5:1 to 1:12, from 4:1 to 1:12, from 3:1 to 1:12, from 2:1 to 1:12, from 1:1 to 1:12, from 8:1 to 1:11, from 7:1 to 1:11, from 6:1 to 1:11, from 5:1 to 1:11, from 4:1 to 1:11, from 3:1 to 1:11, from 2:1 to 1:11, from 1:1 to 1:11, from 8:1 to 1:10, from 7:1 to 1:10, from 6:1 to 1:10, from 5:1 to 1:10, from 4:1 to 1:10, from 3:1 to 1:10, from 2:1 to 1:10, from 1:1 to 1:10, from 8:1 to 1:9, from 7:1 to 1:9, from 6:1 to 1:9, from 5:1 to 1:9, from 4:1 to 1:9, from 3:1 to 1:9, from 2:1 to 1:9, from 1:1 to 1:9, from 8:1 to 1:8, from 7:1 to 1:8, from 6:1 to 1:8, from 5:1 to 1:8, from 4:1 to 1:8, from 3:1 to 1:8, from 2:1 to 1:8, from 1:1 to 1:8, from 8:1 to 1:7, from 7:1 to 1:7, from 6:1 to 1:7, from 5:1 to 1:7, from 4:1 to 1:7, from 3:1 to 1:7, from 2:1 to 1:7, from 1:1 to 1:7, from 8:1 to 1:6, from 7:1 to 1:6, from 6:1 to 1:6, from 5:1 to 1:6, from 4:1 to 1:6, from 3:1 to 1:6, from 2:1 to 1:6, from 1:1 to 1:6, from 8:1 to 1:5, from 7:1 to 1:5, from 6:1 to 1:5, from 5:1 to 1:5, from 4:1 to 1:5, from 3:1 to 1:5, from 2:1 to 1:5, from 1:1 to 1:5, from 8:1 to 1:4, from 7:1 to 1:4, from 6:1 to 1:4, from 5:1 to 1:4, from 4:1 to 1:4, from 3:1 to 1:4, from 2:1 to 1:4, from 1:1 to 1:4, from 8:1 to 1:3, from 7:1 to 1:3, from 6:1 to 1:3, from 5:1 to 1:3, from 4:1 to 1:3, from 3:1 to 1:3, from 2:1 to 1:3, from 1:1 to 1:3, from 8:1 to 1:2, from 7:1 to 1:2, from 6:1 to 1:2, from 5:1 to 1:2, from 4:1 to 1:2, from 3:1 to 1:2, from 2:1 to 1:2, from 1:1 to 1:2, from 8:1 to 1:1, from 7:1 to 1:1, from 6:1 to 1:1, from 5:1 to 1:1, from 4:1 to 1:1, from 3:1 to 1:1, or from 2:1 to 1:1).

In some embodiments, the apolipoprotein to amphipathic molecule molar ratio in the Cargomer ranges from 6:1 to 1:6. In some embodiments, the apolipoprotein to amphipathic molecule molar ratio ranges from 5:1 to 1:6. In some embodiments, the apolipoprotein to amphipathic molecule molar ratio ranges from 4:1 to 1:6. In some embodiments, the apolipoprotein to amphipathic molecule molar ratio ranges from 3:1 to 1:6. In some embodiments, the apolipoprotein to amphipathic molecule molar ratio ranges from 2:1 to 1:6. In some embodiments, the apolipoprotein to amphipathic molecule molar ratio ranges from 5:1 to 1:5. In some embodiments, the apolipoprotein to amphipathic molecule molar ratio ranges from 4:1 to 1:5. In some embodiments, the apolipoprotein to amphipathic molecule molar ratio ranges from 3:1 to 1:5. In some embodiments, the apolipoprotein to amphipathic molecule molar ratio ranges from 2:1 to 1:5. In some embodiments, the apolipoprotein to amphipathic molecule molar ratio ranges from 5:1 to 1:4. In some embodiments, the apolipoprotein to amphipathic molecule molar ratio ranges from 4:1 to 1:4. In some embodiments, the apolipoprotein to amphipathic molecule molar ratio ranges from 3:1 to 1:4. In some embodiments, the apolipoprotein to amphipathic molecule molar ratio ranges from 2:1 to 1:4. In some embodiments, the apolipoprotein to amphipathic molecule molar ratio ranges from 5:1 to 1:3. In some embodiments, the apolipoprotein to amphipathic molecule molar ratio ranges from 4:1 to 1:3. In some embodiments, the apolipoprotein to amphipathic molecule molar ratio ranges from 3:1 to 1:3. In some embodiments, the apolipoprotein to amphipathic molecule molar ratio ranges from 2:1 to 1:3. In some embodiments, the apolipoprotein to amphipathic molecule molar ratio ranges from 5:1 to 1:2. In some embodiments, the apolipoprotein to amphipathic molecule molar ratio ranges from 4:1 to 1:2. In some embodiments, the apolipoprotein to amphipathic molecule molar ratio ranges from 3:1 to 1:2. In some embodiments, the apolipoprotein to amphipathic molecule molar ratio ranges from 2:1 to 1:2. In some embodiments, the apolipoprotein to amphipathic molecule molar ratio ranges from 5:1 to 1:1. In some embodiments, the apolipoprotein to amphipathic molecule molar ratio ranges from 4:1 to 1:1. In some embodiments, the apolipoprotein to amphipathic molecule molar ratio ranges from 3:1 to 1:1. In some embodiments, the apolipoprotein to amphipathic molecule molar ratio ranges from 2:1 to 1:1. In some embodiments, the apolipoprotein to amphipathic molecule molar ratio ranges from 1:1 to 1:6. In some embodiments, the apolipoprotein to amphipathic molecule molar ratio ranges from 1:1 to 1:5. In some embodiments, the apolipoprotein to amphipathic molecule molar ratio ranges from 1:1 to 1:4. In some embodiments, the apolipoprotein to amphipathic molecule molar ratio ranges from 1:1 to 1:3. In some embodiments, the apolipoprotein to amphipathic molecule molar ratio ranges from 1:1 to 1:2. In some embodiments, the apolipoprotein to amphipathic molecule molar ratio ranges from 1:2 to 1:6. In some embodiments, the apolipoprotein to amphipathic molecule molar ratio ranges from 1:2 to 1:5. In some embodiments, the apolipoprotein to amphipathic molecule molar ratio ranges from 1:2 to 1:4. In some embodiments, the apolipoprotein to amphipathic molecule molar ratio ranges from 1:2 to 1:3. In some embodiments, the apolipoprotein to amphipathic molecule molar ratio ranges from 1:3 to 1:6. In some embodiments, the apolipoprotein to amphipathic molecule molar ratio ranges from 1:3 to 1:5. In some embodiments, the apolipoprotein to amphipathic molecule molar ratio ranges from 1:3 to 1:4. In some embodiments, the apolipoprotein to amphipathic molecule molar ratio ranges from 1:4 to 1:6. In some embodiments, the apolipoprotein to amphipathic molecule molar ratio ranges from 1:4 to 1:5. In some embodiments, the apolipoprotein to amphipathic molecule molar ratio ranges from 1:5 to 1:6. In some embodiments, the apolipoprotein to amphipathic molecule molar ratio ranges from 1.5:1 to 1:2. In some embodiments, the apolipoprotein to amphipathic molecule molar ratio ranges from 5:4 to 4:5. In some embodiments, the apolipoprotein to amphipathic molecule molar ratio ranges from 5:3 to 3:5. In some embodiments, the apolipoprotein to amphipathic molecule molar ratio ranges from 5:2 to 2:5. In some embodiments, the apolipoprotein to amphipathic molecule molar ratio ranges from 3:2 to 2:3.

In some embodiments, the ratio of the apolipoprotein molecules to amphipathic molecules is about 1:1. In other embodiments, the ratio of the apolipoprotein molecules to amphipathic molecules is about 1:2. In yet other embodiments, the ratio of the apolipoprotein molecules to amphipathic molecules is about 1:3. In yet other embodiments, the ratio of the apolipoprotein molecules to amphipathic molecules is about 1:4. In yet other embodiments, the ratio of the apolipoprotein molecules to amphipathic molecules is about 1:5. In yet other embodiments, the ratio of the apolipoprotein molecules to amphipathic molecules is about 1:6.

In some embodiments, a Cargomer comprises 1 apolipoprotein molecule.

In other embodiments, a Cargomer comprises 2 apolipoprotein molecules. Cargomers comprising 2 apolipoprotein molecules preferably have a Stokes radius of 3 nm or less. In some embodiments, a Cargomer can comprise 2 apolipoprotein molecules and 1, 2, or 3 negatively charged amphipathic molecules (e.g., negatively charged phospholipid molecules) per apolipoprotein molecule.

In other embodiments, a Cargomer comprises 4 apolipoprotein molecules. Cargomers comprising 4 apolipoprotein molecules preferably have a Stokes radius of 4 nm or less. In some embodiments, a Cargomer can comprise 4 apolipoprotein molecules and 1, 2, or 3 negatively charged amphipathic molecules (e.g., negatively charged phospholipid molecules) per apolipoprotein molecule.

In other embodiments, a Cargomer comprises 8 apolipoprotein molecules. Cargomers comprising 8 apolipoprotein molecules preferably have a Stokes radius of 5 nm or less. In some embodiments, a Cargomer can comprise 8 apolipoprotein molecules and 1, 2, or 3 negatively charged amphipathic molecules (e.g., negatively charged phospholipid molecules) per apolipoprotein molecule. In certain embodiments, the Cargomers of the disclosure do not contain cholesterol and/or a cholesterol derivative (e.g., a cholesterol ester).

In some embodiments, a Cargomer comprises an apolipoprotein to phospholipid ratio in the range of about 1:2 to about 1:3 by weight.

In some embodiments, a Cargomer comprises an apolipoprotein to phospholipid ratio of 1:2.7 by weight.

The Cargomers can be soluble in a biological fluid, for example one or more of lymph, cerebrospinal fluid, vitreous humor, aqueous humor, and blood or a blood fraction (e.g., serum or plasma).

Cargomers may include a targeting functionality, for example to target the Cargomers to a particular cell or tissue type. In some embodiments, the Cargomer includes a targeting moiety attached to an apolipoprotein molecule or an amphipathic molecule. In some embodiments, one or more cargo moieties that are incorporated into the Cargomer has a targeting capability.

### 6.1.4. ApoA-I Formulations

In one aspect, the disclosure relates to an ApoA-I formulation comprising ApoA-I and one or more lipids, wherein the ApoA-I and the lipids are in the form of lipoprotein complexes.

The ApoA-I can be any such apolipoprotein described in Section 6.1.5.1, including, among others, ApoA-I having the amino acid sequence of amino acids 25-267 of SEQ ID NO:2 and/or ApoA-I that is recombinantly expressed.

The lipids can be any one or more of those described in Section 6.1.6.1. The lipids can include neutral lipids and/or negatively charged lipids. The neutral lipids can comprise sphingomyelin or consist of sphingomyelin, such as natural sphingomyelin (e.g., chicken egg sphingomyelin) and/or synthetic sphingomyelin (e.g., palmitoylsphingomyelin). The negatively charged lipids can comprise 1,2-dipalmitoyl-sn-glycero-3-[phospho-rac-(1-glycerol)] (“DPPG”) or a salt thereof, or consist of DPPG or a salt thereof.

The lipids can include neutral phospholipid and negatively charged phospholipid at any weight or molar ratio described herein. The lipids can consist of 95 to 99 weight % neutral phospholipid and 1 to 5 weight % negatively charged phospholipid, such as 96 to 98 weight % neutral phospholipid and 2 to 4 weight % negatively charged phospholipid, or 97 weight % neutral phospholipid and 3 weight % negatively charged phospholipid.

The formulations can comprise ApoA-I and the lipids at any weight or molar ratio described herein. For an example, the molar ratio of the components of the negatively charged lipid to the neutral lipid to the ApoA-I in the formulation is 2-6:90-120:1. Exemplary formulations can include ApoA-I to lipid ratios ranging from 1:2 to 1:3 by weight, such as about 1:2.7 by weight.

Optionally, the formulations can further comprise one or more ophthalmic drugs described at Section 6.1.9.

The formulations can be used in a method of treating an eye disease, such as the methods described at Sections 6.2-6.6. The formulations can also be for use in treating an eye disease. The formulations can also be for use in the manufacture of a medicament for treating an eye disease.

Examples of ApoA-I formulations and uses thereof include those set forth as numbered embodiments 357-458 of Group 1.

### 6.1.5. Lipid Binding Protein Molecules

Lipid binding protein molecules that can be used in the complexes described herein include apolipoproteins such as those described in Section 6.1.5.1 and apolipoprotein mimetic peptides such as those described in Section 6.1.5.2. In some embodiments, the complex comprises a mixture of lipid binding protein molecules. In some embodiments, the complex comprises a mixture of one or more lipid binding protein molecules and one or more apolipoprotein mimetic peptides. In some embodiments, the complex comprises one or more apolipoprotein molecules (e.g., ApoA-I molecules), and not one or more apolipoprotein mimetic peptides.

In some embodiments, the complex comprises 1 to 8 ApoA-I equivalents (e.g., 1, 2, 3, 4, 5, 6, 7, 8, 1 to 7, 1 to 6, 1 to 5, 1 to 4, 1 to 3, 1 to 2, 2 to 8, 2 to 6, 2 to 4, 4 to 6, or 4 to 8 ApoA-I equivalents). Lipid binding proteins can be expressed in terms of ApoA-I equivalents based upon the number of amphipathic helices they contain. For example, ApoA-IM, which typically exists as a disulfide-bridged dimer, can be expressed as 2 ApoA-I equivalents, because each molecule of ApoA-IM contains twice as many amphipathic helices as a molecule of ApoA-I. Conversely, a peptide mimetic that contains a single amphipathic helix can be expressed as a 1/10-1/6 ApoA-I equivalent, because each molecule contains 1/10-1/6 as many amphipathic helices as a molecule of ApoA-I.

### 6.1.5.1. Apolipoproteins

Suitable apolipoproteins that can be included in the lipid binding protein-based complexes include apolipoproteins ApoA-I, ApoA-II, ApoA-IV, ApoA-V, ApoB, ApoC-I, ApoC-II, ApoC-III, ApoD, ApoE, ApoJ, ApoH, and any combination of two or more of the foregoing. Polymorphic forms, isoforms, variants and mutants as well as truncated forms of the foregoing apolipoproteins, the most common of which are Apolipoprotein A-I_{Milano} (ApoA-IM), Apolipoprotein A-I_{Paris} (ApoA-I_{P}), and Apolipoprotein A-I_{Zaragoza} (ApoA-I_{Z}), can also be used. Apolipoproteins mutants containing cysteine residues are also known, and can also be used (see, e.g., U.S. Publication No. 2003/0181372). The apolipoproteins may be in the form of monomers or dimers, which may be homodimers or heterodimers. For example, homo- and heterodimers (where feasible) of ApoA-I (Duverger et al., 1996, Arterioscler. Thromb. Vasc. Biol. 16 (12): 1424-29), ApoA-IM (Franceschini et al., 1985, J. Biol. Chem. 260:1632-35), ApoA-I_{P} (Daum et al., 1999, J. Mol. Med. 77:614-22), ApoA-II (Shelness et al., 1985, J. Biol. Chem. 260 (14): 8637-46; Shelness et al., 1984, J. Biol. Chem. 259 (15): 9929-35), ApoA-IV (Duverger et al., 1991, Euro. J. Biochem. 201 (2): 373-83), ApoE (McLean et al., 1983, J. Biol. Chem. 258 (14): 8993-9000), ApoJ and ApoH may be used.

The apolipoproteins can be modified in their primary sequence to render them less susceptible to oxidations, for example, as described in U.S. Publication Nos. 2008/0234192 and 2013/0137628, and U.S. Pat. Nos. 8,143,224 and 8,541,236. The apolipoproteins can include residues corresponding to elements that facilitate their isolation, such as His tags, or other elements designed for other purposes. Preferably, the apolipoprotein in the complex is soluble in a biological fluid (e.g., lymph, cerebrospinal fluid, vitreous humor, aqueous humor, blood, or a blood fraction (e.g., serum or plasma).

In some embodiments, the complex comprises covalently bound lipid-binding protein monomers, e.g., dimeric apolipoprotein A-I_{Milano}, which is a mutated form of ApoA-I containing a cysteine. The cysteine allows the formation of a disulfide bridge which can lead to the formation of homodimers or heterodimers (e.g., ApoA-I Milano-ApoA-II).

In some embodiments, the apolipoprotein molecules comprise ApoA-I, ApoA-II, ApoA-IV, ApoA-V, ApoB, ApoC-I, ApoC-II, ApoC-III, ApoD, ApoE, ApoJ, or ApoH molecules or a combination thereof.

In some embodiments, the apolipoprotein molecules comprise or consist of ApoA-I molecules. In some embodiments, said ApoA-I molecules are human ApoA-I molecules. In some embodiments, said ApoA-I molecules are recombinant. In some embodiments, the ApoA-I molecules are not ApoA-I_{Milano}.

In some embodiments, the ApoA-I molecules are Apolipoprotein A-I_{Milano} (ApoA-IM), Apolipoprotein A-I_{Paris} (ApoA-IP), or Apolipoprotein A-I_{Zaragoza} (ApoA-IZ) molecules.

Apolipoproteins can be purified from animal sources (and in particular from human sources) or produced recombinantly as is well-known in the art, see, e.g., Chung et al., 1980, J. Lipid Res. 21 (3): 284-91; Cheung et al., 1987, J. Lipid Res. 28 (8): 913-29. See also U.S. Pat. Nos. 5,059,528, 5,128,318, 6,617,134; U.S. Publication Nos. 2002/0156007, 2004/0067873, 2004/0077541, and 2004/0266660; and PCT Publications Nos. WO 2008/104890 and WO 2007/023476. Other methods of purification are also possible, for example as described in PCT Publication No. WO 2012/109162, the disclosure of which is incorporated herein by reference in its entirety.

The apolipoprotein can be in prepro-form, pro-form, or mature form. For example, a complex can comprise ApoA-I (e.g., human ApoA-I) in which the ApoA-I is preproApoA-I, proApoA-I, or mature ApoA-I. In some embodiments, the complex comprises ApoA-I that has at least 90% sequence identity to SEQ ID NO:1:

(SEQ ID NO: 1)
PPQSPWDRVKDLATVYVDVLKDSGRDYVSQFEGSALGKQLNLKLLDNWD
SVTSTFSKLREQLGPVTQEFWDNLEKETEGLRQEMSKDLEEVKAKVQPY
LDDFQKKWQEEMELYRQKVEPLRAELQEGARQKLHELQEKLSPLGEEMR
DRARAHVDALRTHLAPYSDELRQRLAARLEALKENGGARLAEY

In other embodiments, the complex comprises ApoA-I that has at least 95% sequence identity to SEQ ID NO:1. In other embodiments, the complex comprises ApoA-I that has at least 98% sequence identity to SEQ ID NO:1. In other embodiments, the complex comprises ApoA-I that has at least 99% sequence identity to SEQ ID NO:1. In other embodiments, the complex comprises ApoA-I that has 100% sequence identity to SEQ ID NO:1.

In other embodiments, the complex comprises ApoA-I that has at least 95% sequence identity to amino acids 25 to 267 of SEQ ID NO:2. In other embodiments, the complex comprises ApoA-I that has at least 98% sequence identity to amino acids 25 to 267 of SEQ ID NO: 2. In other embodiments, the complex comprises ApoA-I that has at least 99% sequence identity to amino acids 25 to 267 of SEQ ID NO:2. In other embodiments, the complex comprises ApoA-I that has 100% sequence identity to amino acids 25 to 267 of SEQ ID NO:2.

In some embodiments, the complex comprises 1 to 8 apolipoprotein molecules (e.g., 1 to 6, 1 to 4, 1 to 2, 2 to 8, 2 to 6, 2 to 4, 4 to 8, 4 to 6, or 6 to 8 apolipoprotein molecules). In some embodiments, the complex comprises 1 apolipoprotein molecule. In some embodiments, the complex comprises 2 apolipoprotein molecules. In some embodiments, the complex comprises 3 apolipoprotein molecules. In some embodiments, the complex comprises 4 apolipoprotein molecules. In some embodiments, the complex comprises 5 apolipoprotein molecules. In some embodiments, the complex comprises 6 apolipoprotein molecules. In some embodiments, the complex comprises 7 apolipoprotein molecules. In some embodiments, the complex comprises 8 apolipoprotein molecules.

The apolipoprotein molecule(s) can comprise a chimeric apolipoprotein comprising an apolipoprotein and one or more attached functional moieties, such as for example, one or more CRN-001 complex(es), one or more targeting moieties, a moiety having a desired biological activity, an affinity tag to assist with purification, and/or a reporter molecule for characterization or localization studies. An attached moiety with biological activity may have an activity that is capable of augmenting and/or synergizing with the biological activity of a compound or cargo moiety incorporated into a complex of the disclosure. For example, a moiety with biological activity may have antimicrobial (for example, antifungal, antibacterial, anti-protozoal, bacteriostatic, fungistatic, or antiviral) activity. In one embodiment, an attached functional moiety of a chimeric apolipoprotein is not in contact with hydrophobic surfaces of the complex. In another embodiment, an attached functional moiety is in contact with hydrophobic surfaces of the complex. In some embodiments, a functional moiety of a chimeric apolipoprotein may be intrinsic to a natural protein. In some embodiments, a chimeric apolipoprotein includes a ligand or sequence recognized by or capable of interaction with a cell surface receptor or other cell surface moiety.

In one embodiment, a chimeric apolipoprotein includes a targeting moiety that is not intrinsic to the native apolipoprotein, such as for example, S. cerevisiae α-mating factor peptide, folic acid, transferrin, or lactoferrin. In another embodiment, a chimeric apolipoprotein includes a moiety with a desired biological activity that augments and/or synergizes with the activity of a compound or cargo moiety incorporated into a complex of the disclosure. In one embodiment, a chimeric apolipoprotein may include a functional moiety intrinsic to an apolipoprotein. One example of an apolipoprotein intrinsic functional moiety is the intrinsic targeting moiety formed approximately by amino acids 130-150 of human ApoE, which comprises the receptor binding region recognized by members of the low density lipoprotein receptor family. Other examples of apolipoprotein intrinsic functional moieties include the region of ApoB-100 that interacts with the low density lipoprotein receptor and the region of ApoA-I that interacts with scavenger receptor type B 1. In other embodiments, a functional moiety may be added synthetically or recombinantly to produce a chimeric apolipoprotein. Another example is an apolipoprotein with the prepro or pro sequence from another preproapolipoprotein (e.g., prepro sequence from preproapoA-II substituted for the prepro sequence of preproapoA-I). Another example is an apolipoprotein for which some of the amphipathic sequence segments have been substituted by other amphipathic sequence segments from another apolipoprotein.

As used herein, “chimeric” refers to two or more molecules that are capable of existing separately and are joined together to form a single molecule having the desired functionality of all of its constituent molecules. The constituent molecules of a chimeric molecule may be joined synthetically by chemical conjugation or, where the constituent molecules are all polypeptides or analogs thereof, polynucleotides encoding the polypeptides may be fused together recombinantly such that a single continuous polypeptide is expressed. Such a chimeric molecule is termed a fusion protein. A “fusion protein” is a chimeric molecule in which the constituent molecules are all polypeptides and are attached (fused) to each other such that the chimeric molecule forms a continuous single chain. The various constituents can be directly attached to each other or can be coupled through one or more linkers. One or more segments of various constituents can be, for example, inserted in the sequence of an apolipoprotein, or, as another example, can be added N-terminal or C-terminal to the sequence of an apolipoprotein. For example, a fusion protein can comprise an antibody light chain, an antibody fragment, a heavy-chain antibody, or a single-domain antibody.

In some embodiments, a chimeric apolipoprotein is prepared by chemically conjugating the apolipoprotein and the functional moiety to be attached. Means of chemically conjugating molecules are well known to those of skill in the art. Such means will vary according to the structure of the moiety to be attached, but will be readily ascertainable to those of skill in the art. Polypeptides typically contain a variety of functional groups, e.g., carboxylic acid (—COOH), free amino (—NH2), or sulfhydryl (—SH) groups, that are available for reaction with a suitable functional group on the functional moiety or on a linker to bind the moiety thereto. A functional moiety may be attached at the N-terminus, the C-terminus, or to a functional group on an interior residue (i.e., a residue at a position intermediate between the N- and C-termini) of an apolipoprotein molecule. Alternatively, the apolipoprotein and/or the moiety to be tagged can be derivatized to expose or attach additional reactive functional groups.

In some embodiments, fusion proteins that include a polypeptide functional moiety are synthesized using recombinant expression systems. Typically, this involves creating a nucleic acid (e.g., DNA) sequence that encodes the apolipoprotein and the functional moiety such that the two polypeptides will be in frame when expressed, placing the DNA under the control of a promoter, expressing the protein in a host cell, and isolating the expressed protein.

A nucleic acid encoding a chimeric apolipoprotein can be incorporated into a recombinant expression vector in a form suitable for expression in a host cell. As used herein, an “expression vector” is a nucleic acid which, when introduced into an appropriate host cell, can be transcribed and translated into a polypeptide. The vector may also include regulatory sequences such as promoters, enhancers, or other expression control elements (e.g., polyadenylation signals). Such regulatory sequences are known to those skilled in the art (see, e.g., Goeddel, 1990, Gene Expression Technology: Meth. Enzymol. 185, Academic Press, San Diego, Calif.; Berger and Kimmel, Guide to Molecular Cloning Techniques, Methods in Enzymology 152 Academic Press, Inc., San Diego, Calif.; Sambrook et al., 1989, Molecular Cloning—A Laboratory Manual (2nd ed.) Vol. 1-3, Cold Spring Harbor Laboratory, Cold Spring Harbor Press, NY, etc.).

In some embodiments, an apolipoprotein has been modified such that when the apolipoprotein is incorporated into a complex of the disclosure, the modification will increase stability of the complex, confer targeting ability or increase capacity. In one embodiment, the modification includes introduction of cysteine residues into apolipoprotein molecules to permit formation of intramolecular or intermolecular disulfide bonds, e.g., by site-directed mutagenesis. In another embodiment, a chemical crosslinking agent is used to form intermolecular links between apolipoprotein molecules to enhance stability of the complex. Intermolecular crosslinking prevents or reduces dissociation of apolipoprotein molecules from the complex and/or prevents displacement by endogenous apolipoprotein molecules within an individual to whom the complexes are administered. In other embodiments, an apolipoprotein is modified either by chemical derivatization of one or more amino acid residues or by site directed mutagenesis, to confer targeting ability to or recognition by a cell surface receptor.

Complexes can be targeted to a specific cell surface receptor by engineering receptor recognition properties into an apolipoprotein. For example, complexes may be targeted to a particular cell type known to harbor a particular type of infectious agent, for example by modifying the apolipoprotein to render it capable of interacting with a receptor on the surface of the cell type being targeted. For example, complexes may be targeted to macrophages by altering the apolipoprotein to confer recognition by the macrophage endocytic class A scavenger receptor (SR-A). SR-A binding ability can be conferred to a complex by modifying the apolipoprotein by site directed mutagenesis to replace one or more positively charged amino acids with a neutral or negatively charged amino acid. SR-A recognition can also be conferred by preparing a chimeric apolipoprotein that includes an N- or C-terminal extension having a ligand recognized by SR-A or an amino acid sequence with a high concentration of negatively charged residues. Complexes comprising apoplipoproteins can also interact with apolipoprotein receptors such as, but not limited to, ABCA1 receptors, ABCG1 receptors, Megalin, Cubulin and HDL receptors such as SR-B1.

A complex can comprise a lipid binding protein (e.g., an apolipoprotein molecule) which anchors a cargo moiety to a Cargomer. In some embodiments, the apolipoprotein molecule is coupled to a cargo moiety by a direct bond. In other embodiments, the apolipoprotein molecule is coupled to the cargo moiety by a linker, e.g., as described in Section 6.1.8.

### 6.1.5.2. Apolipoprotein Mimetics

Peptides, peptide analogs, and agonists that mimic the activity of an apolipoprotein (collectively referred to herein as “apolipoprotein peptide mimetics”) can also be used in the complexes described herein, either alone, in combination with one or more other lipid binding proteins. Non-limiting examples of peptides and peptide analogs that correspond to apolipoproteins, as well as agonists that mimic the activity of ApoA-I, ApoA-IM, ApoA-II, ApoA-IV, and ApoE, that are suitable for inclusion in the complexes and compositions described herein are disclosed in U.S. Pat. Nos. 6,004,925, 6,037,323 and 6,046,166 (issued to Dasseux et al.), U.S. Pat. No. 5,840,688 (issued to Tso), U.S. Pat. No. 6,743,778 (issued to Kohno), U.S. Publication Nos. 2004/0266671, 2004/0254120, 2003/0171277 and 2003/0045460 (to Fogelman), U.S. Publication No. 2006/0069030 (to Bachovchin), U.S. Publication No. 2003/0087819 (to Bielicki), U.S. Publication No. 2009/0081293 (to Murase et al.), and PCT Publication No. WO/2010/093918 (to Dasseux et al.), the disclosures of which are incorporated herein by reference in their entireties. These peptides and peptide analogues can be composed of L-amino acid or D-amino acids or mixture of L- and D-amino acids. They may also include one or more non-peptide or amide linkages, such as one or more well-known peptide/amide isosteres. Such apolipoprotein peptide mimetic can be synthesized or manufactured using any technique for peptide synthesis known in the art, including, e.g., the techniques described in U.S. Pat. Nos. 6,004,925, 6,037,323 and 6,046,166.

In some embodiments, the lipid binding protein molecules comprise apolipoprotein peptide mimetic molecules and optionally one or more apolipoprotein molecules such as those described above.

In some embodiments, the apolipoprotein peptide mimetic molecules comprise an ApoA-I peptide mimetic, ApoA-II peptide mimetic, ApoA-IV peptide mimetic, or ApoE peptide mimetic or a combination thereof.

A complex of the disclosure can comprise an apolipoprotein peptide mimetic molecule which anchors a cargo moiety to the complex. In some embodiments, the apolipoprotein peptide mimetic molecule is coupled to the cargo moiety by a direct bond. In other embodiments, the apolipoprotein peptide mimetic molecule is coupled to the cargo moiety by a linker, e.g., as described in Section 6.1.8.

### 6.1.6. Amphipathic Molecules

An amphipathic molecule is a molecule that possesses both hydrophobic (apolar) and hydrophilic (polar) elements. Amphipathic molecules that can be used in complexes described herein include lipids (e.g., as described in Section 6.1.6.1), detergents (e.g., as described in Section 6.1.6.2), fatty acids (e.g., as described in Section 6.1.6.3), and apolar molecules and sterols covalently attached to polar molecules such as, but not limited to, sugars or nucleic acids (e.g., as described in Section 6.1.6.4).

The complexes can include a single class of amphipathic molecule (e.g., a single species of phospholipids or a mixture of phospholipids), or can contain a combination of classes of amphipathic molecules (e.g., phospholipids and detergents). The complex can contain one species of amphipathic molecules or a combination of amphipathic molecules configured to facilitate solubilization of the lipid binding protein molecule(s).

In some embodiments, Apomer and/or Cargomer-based complexes comprise only an amount of amphipathic molecules sufficient to solubilize the lipid binding protein molecules. In other words, an Apomer and/or Cargomer-based complex can comprise the minimum amount of one or more amphipathic molecules necessary to solubilize the lipid binding protein molecules.

In some embodiments, the amphipathic molecules included in comprise a phospholipid, a detergent, a fatty acid, an apolar moiety or sterol covalently attached to a sugar, or a combination thereof (e.g., selected from the types of amphipathic molecules discussed above).

In some embodiments, the amphipathic molecules comprise or consist of phospholipid molecules. In some embodiments, the phospholipid molecules comprise negatively charged phospholipids, neutral phospholipids, positively charged phospholipids or a combination thereof. In some embodiments, the phospholipid molecules contribute a net charge of 1-3 per apolipoprotein molecule in the complex. In some embodiments, the net charge is a negative net charge. In some embodiments, the net charge is a positive net charge. In some embodiments, the phospholipid molecules consist of a combination of negatively charged and neutral phospholipids. In some embodiments, the molar ratio of negatively charge phospholipid to neutral phospholipid ranges from 1:1 to 1:3, for example, about 1:1, about 1:2, or about 1:3. In some embodiments, the molar ratio of negatively charged phospholipid to neutral phospholipid is about 1:1 or about 1:2. In some embodiments, the weight ratio of neutral phospholipids to negatively charged phospholipids ranges from 95:5 to 99:1.

In some embodiments, a complex comprises at least one amphipathic molecule which is an anchor.

In some embodiments, the amphipathic molecules comprise neutral phospholipids and negatively charged phospholipids in a weight ratio of 95:5 to 99:1.

### 6.1.6.1. Lipids

Lipid binding protein-based complexes can include one or more lipids. In various embodiments, one or more lipids can be saturated and/or unsaturated, natural and/or synthetic, charged or not charged, zwitterionic or not. In some embodiments, the lipid molecules (e.g., phospholipid molecules) can together contribute a net charge of 1-3 (e.g., 1-3, 1-2, 2-3, 1, 2, or 3) per lipid binding protein molecule in the complex. In some embodiments, the net charge is negative. In other embodiments, the net charge is positive.

In some embodiments, the lipid comprises a phospholipid. Phospholipids can have two acyl chains that are the same or different (for example, chains having a different number of carbon atoms, a different degree of saturation between the acyl chains, different branching of the acyl chains, or a combination thereof). The lipid can also be modified to contain a fluorescent probe (e.g., as described at avantilipids.com/product-category/products/fluorescent-lipids/). Preferably, the lipid comprises at least one phospholipid.

Phospholipids can have unsaturated or saturated acyl chains ranging from about 6 to about 24 carbon atoms (e.g., 6-20, 6-16, 6-12, 12-24, 12-20, 12-16, 16-24, 16-20, or 20-24). In some embodiments, a phospholipid used in a complex of the disclosure has one or two acyl chains of 12, 14, 16, 18, 20, 22, or 24 carbons (e.g., two acyl chains of the same length or two acyl chains of different length).

Non-limiting examples of acyl chains present in commonly occurring fatty acids that can be included in phospholipids are provided in Table 1, below:

TABLE 1

| Length:Number of | |
|---|---|
| Unsaturations | Common Name |
| 14:0 | myristic acid |
| 16:0 | palmitic acid |
| 18:0 | stearic acid |
| 18:1 cisΔ⁹ | oleic acid |
| 18:2 cisΔ^{9, 12} | linoleic acid |
| 18:3 cisΔ^{9, 12, 15} | linonenic acid |
| 20:4 cisΔ^{5, 8, 11, 14} | arachidonic acid |
| 20:5 cisΔ^{5, 8, 11, 14, 17} | eicosapentaenoic acid |
| | (an omega-3 fatty acid) |

Lipids that can be present in the complexes of the disclosure include, but are not limited to, small alkyl chain phospholipids, egg phosphatidylcholine, soybean phosphatidylcholine, dipalmitoylphosphatidylcholine, dimyristoylphosphatidylcholine, distearoylphosphatidylcholine 1-myristoyl-2-palmitoylphosphatidylcholine, 1-palmitoyl-2-myristoylphosphatidylcholine, 1-palmitoyl-2-stearoylphosphatidylcholine, 1-stearoyl-2-palmitoylphosphatidylcholine, dioleoylphosphatidylcholine dioleophosphatidylethanolamine, dilauroylphosphatidylglycerol phosphatidylcholine, phosphatidylserine, phosphatidylethanolamine, phosphatidylinositol, phosphatidylglycerols, diphosphatidylglycerols such as dimyristoylphosphatidylglycerol, dipalmitoylphosphatidylglycerol, distearoylphosphatidylglycerol, dioleoylphosphatidylglycerol, dimyristoylphosphatidic acid, dipalmitoylphosphatidic acid, dimyristoylphosphatidylethanolamine, dipalmitoylphosphatidylethanolamine, dimyristoylphosphatidylserine, dipalmitoylphosphatidylserine, brain phosphatidylserine, brain sphingomyelin, palmitoylsphingomyelin, dipalmitoylsphingomyelin, egg sphingomyelin, milk sphingomyelin, phytosphingomyelin, distearoylsphingomyelin, dipalmitoylphosphatidylglycerol salt, phosphatidic acid, galactocerebroside, gangliosides, cerebrosides, dilaurylphosphatidylcholine, (1,3)-D-mannosyl-(1,3)diglyceride, aminophenylglycoside, 3-cholesteryl-6′-(glycosylthio) hexyl ether glycolipids, and cholesterol and its derivatives. Synthetic lipids, such as synthetic palmitoylsphingomyelin or N-palmitoyl-4-hydroxysphinganine-1-phosphocholine (a form of phytosphingomyelin) can be used to minimize lipid oxidation.

In some embodiments, a lipid binding protein-based complex includes two types of phospholipids: a neutral lipid, e.g., lecithin and/or sphingomyelin (abbreviated SM), and a charged phospholipid (e.g., a negatively charged phospholipid). A “neutral” phospholipid has a net charge of about zero at physiological pH. In many embodiments, neutral phospholipids are zwitterions, although other types of net neutral phospholipids are known and can be used. In some embodiments, the molar ratio of the charged phospholipid (e.g., negatively charged phospholipid) to neutral phospholipid ranges from 1:1 to 1:3, for example, about 1:1, about 1:2, or about 1:3.

The neutral phospholipid can comprise, for example, one or both of the lecithin and/or SM, and can optionally include other neutral phospholipids. In some embodiments, the neutral phospholipid comprises lecithin, but not SM. In other embodiments, the neutral phospholipid comprises SM, but not lecithin. In still other embodiments, the neutral phospholipid comprises both lecithin and SM. All of these specific exemplary embodiments can include neutral phospholipids in addition to the lecithin and/or SM, but in many embodiments do not include such additional neutral phospholipids.

As used herein, the expression “SM” includes sphingomyelins derived or obtained from natural sources, as well as analogs and derivatives of naturally occurring SMs that are impervious to hydrolysis by LCAT, as is naturally occurring SM. SM is a phospholipid very similar in structure to lecithin, but, unlike lecithin, it does not have a glycerol backbone, and hence does not have ester linkages attaching the acyl chains. Rather, SM has a ceramide backbone, with amide linkages connecting the acyl chains. SM can be obtained, for example, from milk, egg or brain. SM analogues or derivatives can also be used. Non-limiting examples of useful SM analogues and derivatives include, but are not limited to, palmitoylsphingomyelin, N-palmitoyl-4-hydroxysphinganine-1-phosphocholine (a form of phytosphingomyelin), palmitoylsphingomyelin, stearoylsphingomyelin, D-erythro-N-16:0-sphingomyelin and its dihydro isomer, D-erythro-N-16:0-dihydro-sphingomyelin. Synthetic SM such as synthetic palmitoylsphingomyelin or N-palmitoyl-4-hydroxysphinganine-1-phosphocholine (phytosphingomyelin) can be used in order to produce more homogeneous complexes and with fewer contaminants and/or oxidation products than sphingolipids of animal origin. Methods for synthesizing SM are described in U.S. Publication No. 2016/0075634.

Sphingomyelins isolated from natural sources can be artificially enriched in one particular saturated or unsaturated acyl chain. For example, milk sphingomyelin (Avanti Phospholipid, Alabaster, Ala.) is characterized by long saturated acyl chains (i.e., acyl chains having 20 or more carbon atoms). In contrast, egg sphingomyelin is characterized by short saturated acyl chains (i.e., acyl chains having fewer than 20 carbon atoms). For example, whereas only about 20% of milk sphingomyelin comprises C16:0 (16 carbon, saturated) acyl chains, about 80% of egg sphingomyelin comprises C16:0 acyl chains. Using solvent extraction, the composition of milk sphingomyelin can be enriched to have an acyl chain composition comparable to that of egg sphingomyelin, or vice versa.

The SM can be semi-synthetic such that it has particular acyl chains. For example, milk sphingomyelin can be first purified from milk, then one particular acyl chain, e.g., the C16:0 acyl chain, can be cleaved and replaced by another acyl chain. The SM can also be entirely synthesized, by e.g., large-scale synthesis. See, e.g., Dong et al., U.S. Pat. No. 5,220,043, entitled Synthesis of D-erythro-sphingomyelins, issued Jun. 15, 1993; Weis, 1999, Chem. Phys. Lipids 102 (1-2): 3-12. SM can be fully synthetic, e.g., as described in U.S. Publication No. 2014/0275590.

The lengths and saturation levels of the acyl chains comprising a semi-synthetic or a synthetic SM can be selectively varied. The acyl chains can be saturated or unsaturated, and can contain from about 6 to about 24 carbon atoms. Each chain can contain the same number of carbon atoms or, alternatively each chain can contain different numbers of carbon atoms. In some embodiments, the semi-synthetic or synthetic SM comprises mixed acyl chains such that one chain is saturated and one chain is unsaturated. In such mixed acyl chain SMs, the chain lengths can be the same or different. In other embodiments, the acyl chains of the semi-synthetic or synthetic SM are either both saturated or both unsaturated. Again, the chains can contain the same or different numbers of carbon atoms. In some embodiments, both acyl chains comprising the semi-synthetic or synthetic SM are identical. In a specific embodiment, the chains correspond to the acyl chains of a naturally-occurring fatty acid, such as for example oleic, palmitic or stearic acid. In another embodiment, SM with saturated or unsaturated functionalized chains is used. In another specific embodiment, both acyl chains are saturated and contain from 6 to 24 carbon atoms. Non-limiting examples of acyl chains present in commonly occurring fatty acids that can be included in semi-synthetic and synthetic SMs are provided in Table 1, above.

In some embodiments, the SM is palmitoyl SM, such as synthetic palmitoyl SM, which has C16:0 acyl chains, or is egg SM, which includes as a principal component palmitoyl SM.

In a specific embodiment, functionalized SM, such as phytosphingomyelin, is used.

Lecithin can be derived or isolated from natural sources, or it can be obtained synthetically. Examples of suitable lecithins isolated from natural sources include, but are not limited to, egg phosphatidylcholine and soybean phosphatidylcholine. Additional non-limiting examples of suitable lecithins include, dipalmitoylphosphatidylcholine, dimyristoylphosphatidylcholine, distearoylphosphatidylcholine 1-myristoyl-2-palmitoylphosphatidylcholine, 1-palmitoyl-2-myristoylphosphatidylcholine, 1-palmitoyl-2-stearoylphosphatidylcholine, 1-stearoyl-2-palmitoylphosphatidylcholine, 1-palmitoyl-2-oleoylphosphatidylcholine, 1-oleoyl-2-palmitylphosphatidylcholine, dioleoylphosphatidylcholine and the ether derivatives or analogs thereof.

Lecithins derived or isolated from natural sources can be enriched to include specified acyl chains. In embodiments employing semi-synthetic or synthetic lecithins, the identity(ies) of the acyl chains can be selectively varied, as discussed above in connection with SM. In some embodiments of the complexes described herein, both acyl chains on the lecithin are identical. In some embodiments of complexes that include both SM and lecithin, the acyl chains of the SM and lecithin are all identical. In a specific embodiment, the acyl chains correspond to the acyl chains of myristic, palmitic, oleic or stearic acid.

The complexes of the disclosure can include one or more negatively charged phospholipids (e.g., alone or in combination with one or more neutral phospholipids). As used herein, “negatively charged phospholipids” are phospholipids that have a net negative charge at physiological pH. The negatively charged phospholipid can comprise a single type of negatively charged phospholipid, or a mixture of two or more different, negatively charged, phospholipids. In some embodiments, the charged phospholipids are negatively charged glycerophospholipids. Specific examples of suitable negatively charged phospholipids include, but are not limited to, a 1,2-dipalmitoyl-sn-glycero-3-[phospho-rac-(1-glycerol)], a phosphatidylglycerol, a phospatidylinositol, a phosphatidylserine, a phosphatidic acid, and salts thereof (e.g., sodium salts or potassium salts). In some embodiments, the negatively charged phospholipid comprises one or more of phosphatidylinositol, phosphatidylserine, phosphatidylglycerol and/or phosphatidic acid. In a specific embodiment, the negatively charged phospholipid comprises or consists of a salt of a phosphatidylglycerol or a salt of a phosphatidylinositol. In another specific embodiment, the negatively charged phospholipid comprises or consists of 1,2-dipalmitoyl-sn-glycero-3-[phospho-rac-(1-glycerol)], or DPPG, or a salt thereof.

The negatively charged phospholipids can be obtained from natural sources or prepared by chemical synthesis. In embodiments employing synthetic negatively charged phospholipids, the identities of the acyl chains can be selectively varied, as discussed above in connection with SM. In some embodiments of the complexes of the disclosure, both acyl chains on the negatively charged phospholipids are identical. In some embodiments, the acyl chains all types of phospholipids included in a complex of the disclosure are all identical. In a specific embodiment, the complex comprises negatively charged phospholipid(s), and/or SM all having C16:0 or C16:1 acyl chains. In a specific embodiment the fatty acid moiety of the SM is predominantly C16:1 palmitoyl. In one specific embodiment, the acyl chains of the charged phospholipid(s), lecithin and/or SM correspond to the acyl chain of palmitic acid. In yet another specific embodiment, the acyl chains of the charged phospholipid(s), lecithin and/or SM correspond to the acyl chain of oleic acid.

Examples of positively charged phospholipids that can be included in the complexes of the disclosure include N1-[2-((1S)-1-[(3-aminopropyl)amino]-4-[di(3-amino-propyl)amino]butylcarboxamido)ethyl]-3,4-di[oleoyloxy]-benzamide, 1,2-di-O-octadecenyl-3-trimethylammonium propane, 1,2-dimyristoleoyl-sn-glycero-3-ethylphosphocholine, 1-palmitoyl-2-oleoyl-sn-glycero-3-ethylphosphocholine, 1,2-dioleoyl-sn-glycero-3-ethylphosphocholine, 1,2-distearoyl-sn-glycero-3-ethylphosphocholine, 1,2-dipalmitoyl-sn-glycero-3-ethylphosphocholine, 1,2-dimyristoyl-sn-glycero-3-ethylphosphocholine, 1,2-dilauroyl-sn-glycero-3-ethylphosphocholine, 1,2-dilauroyl-sn-glycero-3-ethylphosphocholine, 1,2-dioleoyl-3-dimethylammonium-propane1,2-dimyristoyl-3-dimethylammonium-propane, 1,2-dipalmitoyl-3-dimethylammonium-propane, N-(4-carboxybenzyl)-N,N-dimethyl-2,3-bis(oleoyloxy) propan-1-aminium, 1,2-dioleoyl-3-trimethylammonium-propane, 1,2-dioleoyl-3-trimethylammonium-propane, 1,2-stearoyl-3-trimethylammonium-propane, 1,2-dipalmitoyl-3-trimethylammonium-propane, 1,2-dimyristoyl-3-trimethylammonium-propane, N-[1-(2,3-dimyristyloxy) propyl]-N, N-dimethyl-N-(2-hydroxyethyl) ammonium bromide, N,N, N-trimethyl-2-bis[(1-oxo-9-octadecenyl)oxy]-(Z,Z)-1propanaminium methyl sulfate, and salts thereof (e.g., chloride or bromide salts).

The lipids used are preferably at least 95% pure, and/or have reduced levels of oxidative agents (such as but not limited to peroxides). Lipids obtained from natural sources preferably have fewer polyunsaturated fatty acid moieties and/or fatty acid moieties that are not susceptible to oxidation. The level of oxidation in a sample can be determined using an iodometric method, which provides a peroxide value, expressed in milli-equivalent number of isolated iodines per kg of sample, abbreviated meq O/kg. See, e.g., Gray, 1978, Measurement of Lipid Oxidation: A Review, Journal of the American Oil Chemists Society 55:539-545; Heaton, F. W. and Ur, Improved lodometric Methods for the Determination of Lipid Peroxides, 1958, Journal of the Science of Food and Agriculture 9:781-786. Preferably, the level of oxidation, or peroxide level, is low, e.g., less than 5 meq O/kg, less than 4 meq O/kg, less than 3 meq O/kg, or less than 2 meq O/kg.

Complexes can in some embodiments include small quantities of additional lipids. Virtually any type of lipids can be used, including, but not limited to, lysophospholipids, galactocerebroside, gangliosides, cerebrosides, glycerides, triglycerides, and sterols and sterol derivatives (e.g., a plant sterol, an animal sterol, such as cholesterol, or a sterol derivative, such as a cholesterol derivative). For example, a complex of the disclosure can contain cholesterol or a cholesterol derivative, e.g., a cholesterol ester. The cholesterol derivative can also be a substituted cholesterol or a substituted cholesterol ester. The complexes of the disclosure can also contain an oxidized sterol such as, but not limited to, oxidized cholesterol or an oxidized sterol derivative (such as, but not limited to, an oxidized cholesterol ester). In some embodiments, the complexes do not include cholesterol and/or its derivatives (such as a cholesterol ester or an oxidized cholesterol ester).

### 6.1.6.2. Detergents

The complexes can contain one or more detergents. The detergent can be zwitterionic, nonionic, cationic, anionic, or a combination thereof. Exemplary zwitterionic detergents include 3-[(3-Cholamidopropyl)dimethylammonio]-1-propanesulfonate (CHAPS), 3-[(3-Cholamidopropyl)dimethylammonio]-2-hydroxy-1-propanesulfonate (CHAPSO), and N,N-dimethyldodecylamine N-oxide (LDAO). Exemplary nonionic detergents include D-(+)-trehalose 6-monooleate, N-octanoyl-N-methylglucamine, N-nonanoyl-N-methylglucamine, N-decanoyl-N-methylglucamine, 1-(7Z-hexadecenoyl)-rac-glycerol, 1-(8Z-hexadecenoyl)-rac-glycerol, 1-(8Z-heptadecenoyl)-rac-glycerol, 1-(9Z-hexadecenoyl)-rac-glycerol, 1-decanoyl-rac-glycerol. Exemplary cationic detergents include(S)-O-methyl-serine dodecylamide hydrochloride, dodecylammonium chloride, decyltrimethylammonium bromide, and cetyltrimethylammonium sulfate. Exemplary anionic detergents include cholesteryl hemisuccinate, cholate, alkyl sulfates, and alkyl sulfonates.

### 6.1.6.3. Fatty Acids

The complexes can contain one or more fatty acids. The one or more fatty acids can include short-chain fatty acids having aliphatic tails of five or fewer carbons (e.g. butyric acid, isobutyric acid, valeric acid, or isovaleric acid), medium-chain fatty acids having aliphatic tails of 6 to 12 carbons (e.g., caproic acid, caprylic acid, capric acid, or lauric acid), long-chain fatty acids having aliphatic tails of 13 to 21 carbons (e.g., myristic acid, palmitic acid, stearic acid, or arachidic acid), very long chain fatty acids having aliphatic tails of 22 or more carbons (e.g., behenic acid, lignoceric acid, or cerotic acid), or a combination thereof. The one or more fatty acids can be saturated (e.g., caprylic acid, capric acid, lauric acid, myristic acid, palmitic acid, stearic acid, arachidic acid, behenic acid, lignoceric acid, or cerotic acid), unsaturated (e.g., myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, or docosahexaenoic acid) or a combination thereof. Unsaturated fatty acids can be cis or trans fatty acids. In some embodiments, unsaturated fatty acids used in the complexes of the disclosure are cis fatty acids.

### 6.1.6.4. Apolar Molecules and Sterols Attached to a Sugar

The complexes can contain one or more amphipathic molecules that comprise an apolar molecule or moiety (e.g., a hydrocarbon chain, an acyl or diacyl chain) or a sterol (e.g., cholesterol) attached to a sugar (e.g., a monosaccharide such as glucose or galactose, or a disaccharide such as maltose or trehalose). The sugar can be a modified sugar or a substituted sugar. Exemplary amphipathic molecules comprising an apolar molecule attached to a sugar include dodecan-2-yloxy-ß-D-maltoside, tridecan-3-yloxy-ß-D-maltoside, tridecan-2-yloxy-ß-D-maltoside, n-dodecyl-ß-D-maltoside (DDM), n-octyl-ß-D-glucoside, n-nonyl-ß-D-glucoside, n-decyl-ß-D-maltoside, n-dodecyl-ß-D-maltopyranoside, 4-n-Dodecyl-α,α-trehalose, 6-n-dodecyl-α,α-trehalose, and 3-n-dodecyl-α,α-trehalose.

In some embodiments, the apolar moiety is an acyl or a diacyl chain.

In some embodiments, the sugar is a modified sugar or a substituted sugar.

### 6.1.7. Anchors

A cargo moiety can be covalently bound to an amphipathic or apolar moiety to facilitate coupling of the cargo moiety to a lipid binding protein-based complex. Amphipathic and apolar moieties can interact with apolar regions in lipid binding protein-based complexes, thereby anchoring cargo moieties attached to amphipathic and apolar moieties to the complexes.

Amphipathic moieties that can be used as anchors include lipids (e.g., as described in Section 6.1.6.1) and fatty acids (e.g., as described in Section 6.1.6.3). In some embodiments, the anchors comprise a sterol or a sterol derivative e.g., a plant sterol, an animal sterol, or a sterol derivative such as a vitamin). For example, sterols such as cholesterol can be covalently bound to a cargo moiety (e.g., via the hydroxyl group at the 3-position of the A-ring of the sterol) and used to anchor a cargo moiety to a complex. Apolar moieties that can be used as anchors include alkyl chains, acyl chains, and diacyl chains. Cargo moieties can be covalently bound to anchor moieties directly or indirectly via a linker (e.g., via a difunctional peptide or other linker described in Section 6.1.8). Cargo moieties that are biologically active may retain their biological activity while covalently bound to the anchor (or linker attached to the anchor), while others may require cleavage of the covalent bond (e.g., by hydrolysis) attaching the cargo moiety to the anchor (or linker attached to the anchor) to regain biological activity.

In some embodiments, at least one cargo moiety is coupled to an anchor. In some embodiments, the anchor comprises an amphipathic and/or apolar moiety. In some embodiments, the anchor comprises an amphipathic moiety. In some embodiments, the amphipathic moiety comprises one of the amphipathic molecules in the complex. In some embodiments, the amphipathic moiety comprises a lipid, a detergent, a fatty acid, an apolar molecule attached to a sugar, or a sterol attached to a sugar.

In some embodiments, the amphipathic moiety comprises a sterol. In some embodiments, the sterol comprise an animal sterol or a plant sterol. In some embodiments, the sterol comprises cholesterol.

In other embodiments, the anchor comprises an apolar moiety. In some embodiments, the apolar moiety comprises an alkyl chain, an acyl chain, or a diacyl chain.

In some embodiments, a cargo moiety is coupled to the anchor by a direct bond.

In some embodiments, a cargo moiety is coupled to the anchor by a linker.

### 6.1.8. Linkers

Linkers comprise a chain of atoms that covalently attach cargo moieties to other moieties in a cargo-carrying complex such as a Cargomer, for example to apolipoprotein molecules, amphipathic molecules, and anchors. A number of linker molecules are commercially available, for example from ThermoFisher Scientific. Suitable linkers are well known to those of skill in the art and include, but are not limited to, straight or branched-chain carbon linkers, heterocyclic carbon linkers, and peptide linkers. A linker can be a bifunctional linker, which is either homobifunctional or heterobifunctional.

Suitable linkers include cleavable and non-cleavable linkers.

A linker may be a cleavable linker, facilitating release of a cargo moiety in vivo. Cleavable linkers include acid-labile linkers (e.g., comprising hydrazine or cis-aconityl), protease-sensitive (e.g., peptidase-sensitive) linkers, photolabile linkers, or disulfide-containing linkers (Chari et al., 1992, Cancer Research 52:127-131; U.S. Pat. No. 5,208,020). A cleavable linker is typically susceptible to cleavage under intracellular conditions. Suitable cleavable linkers include, for example, a peptide linker cleavable by an intracellular protease, such as lysosomal protease or an endosomal protease. In exemplary embodiments, the linker can be a dipeptide linker, such as a valine-citrulline (val-cit) or a phenylalanine-lysine (phe-lys) linker.

A cleavable linker can be pH-sensitive, i.e., sensitive to hydrolysis at certain pH values. Typically, a pH-sensitive linker is hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used. (See, e.g., U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661). Such linkers are relatively stable under neutral pH conditions, such as those in the blood, but are unstable at below pH 5.5 or 5.0, the approximate pH of the lysosome. In certain embodiments, the hydrolyzable linker is a thioether linker (such as, e.g., a thioether attached to the cargo moiety via an acylhydrazone bond (see, e.g., U.S. Pat. No. 5,622,929).

In some embodiments, the linker is cleavable under reducing conditions (e.g., a disulfide linker). A variety of disulfide linkers are known in the art, including, for example, those that can be formed using SATA (N-succinimidyl-5-acetylthioacetate), SPDP (N-succinimidyl-3-(2-pyridyldithio) propionate), SPDB (N-succinimidyl-3-(2-pyridyldithio) butyrate) and SMPT (N-succinimidyloxycarbonyl-alpha-methyl-alpha-(2-pyridyl-dithio) toluene), SPDB and SMPT (see, e.g., Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987. See also, U.S. Pat. No. 4,880,935).

In some embodiments, the linker is cleavable by a cleaving agent, e.g., an enzyme, that is present in the intracellular environment (e.g., within a lysosome or endosome or caveolea). The linker can be, e.g., a peptidyl linker that is cleaved by an intracellular peptidase or protease enzyme, including, but not limited to, a lysosomal or endosomal protease. In some embodiments, the peptidyl linker is at least two amino acids long or at least three amino acids long. Cleaving agents can include cathepsins B and D and plasmin, all of which are known to hydrolyze dipeptide drug derivatives resulting in the release of active drug inside target cells (see, e.g., Dubowchik and Walker, 1999, Pharm. Therapeutics 83:67-123). In some embodiments, the peptidyl linker cleavable by an intracellular protease is a Val-Cit linker or a Phe-Lys linker.

In some embodiments, the linker is a malonate linker (Johnson et al., 1995, Anticancer Res. 15:1387-93), a maleimidobenzoyl linker (Lau et al., 1995, Bioorg-Med-Chem. 3 (10): 1299-1304), or a 3′-N-amide analog (Lau et al., 1995, Bioorg-Med-Chem. 3 (10): 1305-12).

In other embodiments, the linker unit is not cleavable and the cargo moiety is released, for example, by complex degradation. Exemplary non-cleavable linkers include maleimidocaproyl, N-succinimidyl 4-(maleimidomethyl)cyclohexanecarboxylate (SMCC) and N-succinimidyl-4-(iodoacetyl)-aminobenzoate (SIAB).

In some embodiments, a cargo moeity is coupled to an anchor (e.g., as described in Section 6.1.7) by a linker. In some embodiments, the linker coupling the cargo moiety to the anchor is a bifunctional linker. In some embodiments, the linker coupling the cargo moiety to the anchor is a cleavable linker. In some embodiments, the cleavable linker is a dipeptide linker such as a valine-citrulline (val-cit) or a phenylalanine-lysine (phe-lys) linker. In some embodiments, the linker coupling the cargo moiety to the anchor is a non-cleavable linker. Exemplary non-cleavable linkers include maleimidocaproyl, N-succinimidyl 4-(maleimidomethyl)cyclohexanecarboxylate (SMCC) and N-succinimidyl-4-(iodoacetyl)-aminobenzoate (SIAB).

### 6.1.9. Ophthalmic Drugs

In some embodiments of the methods described herein, a lipid binding protein-based complex (e.g., CER-001) can be used as a carrier to deliver one or more ophthalmic drugs to a subject's eye. In some embodiments, the one or more ophthalmic drugs can be considered cargo moieties, and can be complexed to a lipid binding protein-based complex (e.g., CER-001) either non-covalently or covalently to a component of the complex (e.g., via an anchor or linker). In some embodiments, the one or more ophthalmic drugs are not covalently linked to the complex. One or more ophthalmic drugs can be added to a pre-formed complex, e.g., pre-formed CER-001, to make a complex which further comprises the one or more ophthalmic drugs. Complexation between the one or more ophthalmic drugs and a pre-formed complex can be promoted by performing one or more heating and cooling cycles, for example as described in Example 1. Alternatively, one or more ophthalmic drugs can be included during the process used to make a complex, e.g., included in a starting suspension comprising lipid binding protein and lipid components subjected to thermal cycling. Thermal cycling processes for making lipid binding protein-based complexes are described in WO 2012/109162 and WO/2019/030574.

In some embodiments, the one or more ophthalmic drugs comprise a steroid, a kinase inhibitor, an angiotensin II receptor antagonist, an aldose reductase inhibitor, an immunosuppressant, a carbonic anhydrase inhibitor, an antimicrobial agent, an antiviral agent, an antihistamine, an anti-inflammatory, a prostaglandin analog, or a combination thereof.

Exemplary ophthalmic drugs that can be used include but are not limited to, steroids such as dexamethasone, dexamethasone palmitate, difluprednate, estradiol, fluocinolone, fluorometholone, hydrocortisone, loteprednol etabonate, prednisolone, triamcinolone, rimexolone, and spironolactone; kinase inhibitors such as axitinib, BMS-794833 (N-(4-((2-amino-3-chloropyridin-4-yl)oxy)-3-fluorophenyl)-5-(4-fluorophenyl)-4-oxo-1,4-dihydropyridine-3-carboxamide), carbozantinib, cediranib, dovitinib, lapatinib, lenvatinib, motesanib, nintedanib, orantinib, PD173074 (N-[2-[[4-(Diethylamino)butyl]amino]-6-(3,5-dimethoxyphenyl) pyri-do[2,3-d]pyrimidin-7-yl]-N′-(1,1-dimethylethyl) urea), pazopanib, regorafenib, sorafenib, tofacitinib, and ZM323881 (5-((7-Benzyloxyquinazolin-4-yl)amino)-4-fluoro-2-methylphenol); angiotensin II receptor antagonists such as candesartan, irbesartan, losartan, olmesartan, telmisartan, and valsartan; aldose reductase inhibitors such as 2-methylsorbinol; immunosuppressants such as sirolimus, cyclosporine and tacrolimus; carbonic anhydrase inhibitors such as acetazolamide, brinzolamide, dorzolamide, ethoxzolamide and methazolamide; antimicrobial, antifungal and antiviral agents such as azithromycin, acyclovir, chloramphenicol, chlortetracycline, ciprofloxacin, fusidic acid, gancyclovir, norfloxacin, ofloxacin, tetracycline, and zidovudine; antihistamines such as levocabastine; non-steroidal anti-inflammatory drugs such as bromfenac, diclofenac, indomethacin, and nepafenac; and prostaglandin analogs such as latanoprost, travaprost, bimatoprost, and tafluprost.

In some embodiments, a lipid binding protein based-complex includes a prostaglandin analog such as latanoprost, travaprost, bimatoprost, tafluprost, or a combination thereof. In a specific embodiment, a lipid binding protein based-complex includes latanoprost. In another embodiment, a lipid binding protein-based complex includes travaprost. In another embodiment, a lipid binding protein-based complex includes bimatoprost. In another embodiment, a lipid binding protein-based complex includes tafluprost.

In some embodiments, a lipid binding protein based-complex includes dexamethasone, axitinib, cediranib, dovitinib, motesanib, pazopanib, regorafenib, losartan, olmesartan, dorzolamide, diclofenac, nepafenac, or a combination thereof.

In other embodiments, a lipid binding protein based-complex includes azithromycin.

In other embodiments, a lipid binding protein based-complex includes spironolactone.

In other embodiments, a lipid binding protein based-complex includes dexamethasone palmitate.

In other embodiments, a lipid binding protein based-complex includes cyclosporine.

In other embodiments, a lipid binding protein based-complex includes dexamethasone.

In other embodiments, a lipid binding protein based-complex includes loteprednol etabonate.

In other embodiments, a lipid binding protein based-complex includes triamcinolone.

In other embodiments, a lipid binding protein based-complex includes acyclovir.

In other embodiments, a lipid binding protein based-complex includes pazopanib.

In other embodiments, a lipid binding protein based-complex includes sirolimus.

In other embodiments, a lipid binding protein based-complex includes tacrolimus.

In other embodiments, a lipid binding protein based-complex includes nepafenac.

### 6.1.9.1. CER-001 and Ophthalmic Drug Combinations

As disclosed herein, CER-001 can be used as a drug carrier to deliver one or more ophthalmic drugs to a subject's eye. Accordingly, the disclosure provides compositions comprising CER-001 and one or more ophthalmic drugs, e.g., one or more drugs which are hydrophobic and/or poorly water soluble or insoluble.

In one embodiment, the composition comprises CER-001 and a steroid. In some embodiments, the composition comprises CER-001 and dexamethasone. In some embodiments, the composition comprises CER-001 and dexamethasone palmitate. In some embodiments, the composition comprises CER-001 and loteprednol etabonate. In some embodiments, the composition comprises CER-001 and triamcinolone.

In another embodiment, the composition comprises CER-001 and an antimicrobial, antifungal, or antiviral agent. In some embodiments, the composition comprises CER-001 and azithromycin. In some embodiments, the composition comprises CER-001 and acyclovir.

In another embodiment, the composition comprises CER-001 and a prostaglandin analog. In some embodiments, the composition comprises CER-001 and latanoprost. In some embodiments, the composition comprises CER-001 and travaprost. In some embodiments, the composition comprises CER-001 and bimatoprost. In some embodiments, the composition comprises CER-001 and tafluprost.

In another embodiment, the composition comprises CER-001 and a kinase inhibitor. In some embodiments, the composition comprises CER-001 and pazopanib.

In another embodiment, the composition comprises CER-001 and an immunosuppressant. In some embodiments, the composition comprises CER-001 and sirolimus. In some embodiments, the composition comprises CER-001 and tacrolimus.

In another embodiment, the composition comprises CER-001 and non-steroidal anti-inflammatory drugs. In some embodiments, the composition comprises CER-001 and nepafenac.

In a further embodiment, the composition comprises CER-001 and spironolactone.

In another embodiment, the composition comprises CER-001 and cyclosporin.

Compositions described in this Section 6.1.9.1 can be prepared by any suitable means, for example as described in Section 6.1.10, e.g., by thermal cycling a mixture comprising CER-001 and the ophthalmic drug(s). Compositions can be suitably formulated for the intended route of administration such as local administration for example topical administration or intraocular administration. Compositions for intraocular administration can be formulated for administration by, for example, intraocular injection, for example intra-vitreal injection, sub-conjunctival injection, parabulbar injection, peribulbar injection retro-bulbar injection, suprachoroidal injection, or suprascleral injection. For topical administration, the composition may be formulated for administration, for example, as an eye drop. Compositions can also be formulated as an implant, formulated for administration via iontophoresis, or formulated for administration via electroporation. In some embodiments, the composition is formulated for suprachoroidal injection. In other embodiments, the composition is formulated for suprascleral injection. In other embodiments, the composition is formulated as an implant (e.g., a disc, sheet, plug, rod, or pellet). In other embodiments, the composition is formulated for administration via iontophoresis. In other embodiments, the composition is formulated for administration via electroporation.

### 6.1.10. Formulations

Lipid binding protein-based complexes can be formulated for the intended route of administration, for example according to techniques known in the art (e.g., as described in Allen et al., eds., 2012, Remington: The Science and Practice of Pharmacy, 22nd Edition, Pharmaceutical Press, London, UK; or as described in Davis et al., 2004, Curr Opin Mol Ther. 6 (2): 195-205). In some embodiments, a formulation comprises a lipid binding protein-based complex, such as CER-001, and one or more ophthalmic drugs, e.g., one or more ophthalmic drugs described in Section 6.1.9.

In some embodiments, a formulation of a lipid binding protein-based complex, such as CER-001, and optionally one or more ophthalmic drugs is formulated for delivery via intraocular injection, for example intra-vitreal injection, sub-conjunctival injection, parabulbar injection, peribulbar injection retro-bulbar injection, suprachoroidal injection, or suprascleral injection.

In some embodiments, a formulation is formulated for suprachoroidal injection. Suprachoroidal injection can be used to achieve higher chorioretinal drug concentration than may be achieved by traditional intravitreal injection. In other embodiments, a formulation is formulated for suprascleral injection.

In some embodiments, a formulation of a lipid binding protein-based complex, such as CER-001, and optionally one or more ophthalmic drugs is formulated for delivery as an implant. Implants of the disclosure can have various shapes, for example a disc, sheet, plug, rod, or pellet. Implants can be biodegradable. Biodegradable implants can be formulated with substances such as polylactic acid and/or poly-lactic-glycolic-acid, which degrade over time. Alternatively, implants can be non-biodegradable. Non-biodegradable implants can be formulated with substances such as silicon or a polymer such as ethylene vinyl acetate or polyvinyl alcohol.

Implants can be implanted, for example, into the episcleral or intrascleral space, at the sclera, into the vitreous cavity, or anterior chamber. Subconjunctival, intrascleral, and anterior chamber implants can be used, for example, in the treatment of anterior-segment diseases, and intravitreal, suprachoroidal, and intrascleral implants can be used, for example, to treat posterior-segment diseases.

In some embodiments, a formulation of a lipid binding protein-based complex, such as CER-001, and optionally one or more ophthalmic drugs is formulated for delivery via iontophoresis, for example transcorneal or transscleral iontophoresis.

In other embodiments, a formulation of a lipid binding protein-based complex, such as CER-001, and optionally one or more ophthalmic drugs is formulated for delivery via electroporation.

CER-001 intended for administration by infusion can be formulated in a phosphate buffer with sucrose and mannitol excipients, for example as described in WO 2012/109162. Formulations of lipid binding protein-based complexes intended for topical administration can include, for example, carriers, stabilizers, excipients, and combinations thereof. A topical formulation (e.g., eye drops) can include buffers such as phosphate, citrate or other inorganic acid buffers, antioxidants such as ascorbic acid and/or methionine, preservatives, low molecular weight polypeptides, proteins such as gelatin, serum albumin or immunoglobulin, hydrophilic polymers such as PVP, amino acids, monosaccharides or disaccharides or other carbohydrates, chelating agents, sugars, non-ionic surfactants and the like.

In some embodiments, a topical formulation comprises an osmolarity adjusting agent. In some embodiments, the osmolarity adjusting agent is sodium chloride.

In some embodiments, a topical formulation comprises a preservative. In some embodiments, the preservative is benzalkonium chloride, cetrimonium, sodium perborate, stabilized oxychloro complex, SofZia, polyquaternium-1, chlorobutanol, edetate disodium, polyhexamethylene biguanide, or a combination thereof.

In some embodiments, a topical formulation comprises a buffer agent. In some embodiments, the buffer agent is selected from borates, borate-polyol complexes, succinate, phosphate buffering agents, citrate buffering agents, acetate buffering agents, carbonate buffering agents, organic buffering agents, amino acid buffering agents, and combinations thereof.

In some embodiments, a topical formulation comprises a tonicity adjusting agent. In some embodiments, the tonicity adjusting agent is selected from sodium chloride, sodium nitrate, sodium sulfate, sodium bisulfate, potassium chloride, calcium chloride, magnesium chloride, zinc chloride, potassium acetate, sodium acetate, sodium bicarbonate, sodium carbonate, sodium thiosulfate, magnesium sulfate, disodium hydrogen phosphate, sodium dihydrogen phosphate, potassium dihydrogen phosphate, dextrose, mannitol, sorbitol, dextrose, sucrose, urea, propylene glycol, glycerin, trehalose, and combinations thereof.

Formulations of lipid binding protein-based complexes, e.g., CER-001, intended for intraocular administration can include, for example, carriers, stabilizers, viscosifiers, osmolarity adjusting agents, buffers, and combinations thereof. In some embodiments, an intraocular formulation comprises an osmolarity adjusting agent. An example of osmolarity adjusting agent is sodium chloride. In some embodiments, an intraocular formulation comprises a buffer agent. Examples of buffer agents include borates, borate-polyol complexes, succinate, phosphate buffering agents, citrate buffering agents, acetate buffering agents, carbonate buffering agents, organic buffering agents, amino acid buffering agents, and combinations thereof.

In some embodiments, a formulation comprising CER-001 (optionally where the CER-001 is used as a carrier for one or more ophthalmic drugs) can comprise CER-001 at a concentration of 0.5 mg/ml to 8 mg/ml on a protein weight basis (e.g., 0.5 mg/ml, 0.8 mg/ml, 1 mg/ml, 2 mg/ml, 3 mg/ml, 4 mg/ml, 5 mg/ml, 6 mg/ml, 7 mg/ml, 8 mg/ml, any range bounded by any two of the foregoing values). In some embodiments, a formulation comprising CER-001 can comprise CER-001 at a concentration of at least 1 mg/ml, at least 2 mg/ml, at least 3 mg/ml, at least 4 mg/ml, at least 5 mg/ml, at least 6 mg/ml, at least 7 mg/ml, or at least 8 mg/ml (on a protein weight basis).

Formulations of a lipid binding protein-based complex (e.g., CER-001) and one or more ophthalmic drugs can be produced, for example, by thermal cycling a mixture comprising the lipid binding protein-based complex and the one or more ophthalmic drugs. For example, the mixture can be (a) heated from a temperature in a first temperature range to a temperature in a second temperature range, then (b) cooled from a temperature in the second temperature range to a temperature in the first temperature range, then (c) optionally subjected to one or more additional heating and cooling cycles, e.g., for a total of two, three, four, five, or six heating and cooling cycles. Alternatively, the mixture can be (a) cooled from a temperature in the second temperature range to a temperature in the first temperature range, then (b) heated from a temperature in the first temperature range to a temperature in the second temperature range, then (c) optionally subjected to one or more additional cooling and heating cycles, e.g., for a total of two, three, four, five, or six cooling and heating cycles. The first temperature range can in some embodiments include temperatures from 30° C. to 45° C. (e.g., 35° C. to 45° C., 30° C. to 35° C., 35° C. to 40° C., or 40° C. to 45° C.). The second temperature range can in some embodiments include temperatures from 50° C. to 65° C. (e.g., 50° C. to 60° C., 50° C. to 55° C., or 55° C. to 60° C.). In some embodiments, the thermal cycling comprises thermal cycling between 37° C. and 55° C., for example as described in Example 1. Accordingly, in some aspects, the disclosure provides compositions comprising a lipid binding protein-based complex, such as CER-001, and one or more ophthalmic drugs produced by a process comprising thermal cycling a mixture comprising the lipid binding protein-based complex and one or more ophthalmic drugs.

### 6.2. Subject Populations

Subjects who can be treated according to the methods described herein are preferably mammals, most preferably human.

In some aspects, the subject can be a subject in need of therapy for an eye disease, for example an eye disease associated with lipid accumulation, e.g., ocular lipid deposits. In some cases the lipids may accumulate in the eye or near the eye. Exemplary eye diseases associated with lipid accumulation that can be treated by the methods of the disclosure include cataract, dry eye disease, such as dry eye disease associated with Meibomian gland dysfunction or lacrimal gland dysfunction, blepharitis, uveitis, diseases of the cornea such as lipid keratopathy (e.g., secondary lipid keratopathy, for example lipid keratopathy secondary to previous ocular disease or injury) and corneal dystrophy (e.g., an inherited corneal dystrophy, an anterior or superficial corneal dystrophy, a stromal corneal dystrophy, or a posterior corneal dystrophy), eye diseases associated with LCAT deficiency such as fish-eye disease, dry macular degeneration (dry AMD), Stargardt disease and Leber's idiopathic stellate neuroretinitis. Lipid deposits in the cornea, for example, in subjects with LCAT deficiency, can cause impairment, e.g., blurred vision.

In some aspects, the subject has a cataract. For clarity, treatment of a subject having a cataract encompasses treatment of subjects having a cataract in one eye as well as subjects having a cataract in both eyes.

In some aspects, the subject has an inflammatory eye disease such as uveitis (e.g., anterior uveitis, intermediate uveitis, posterior uveitis, or panuveitis) or scleritis. In some aspects, the subject has acute anterior uveitis.

In some embodiments the subject treated according to the methods and/or dosing regimens of the disclosure has an LCAT deficiency, optionally wherein the lipid binding protein-based complex used to treat the subject is CER-001. The subject can be homozygous or heterozygous for a LCAT mutation. In some embodiments, the subject treated according to the methods and/or dosing regimens of the disclosure has fish-eye disease. Subjects with fish-eye disease typically develop bilateral corneal opacity and can have visual impairment, e.g., decreased contrast sensitivity compared to normal and/or blurred vision. Corneal opacity and its progression or regression (e.g., in response to treatment as described herein) can be qualitatively evaluated, for example by comparing slit lamp images of a subject's eyes taken at different times. Corneal opacity can also be evaluated quantitatively, for example by anterior segment optical coherence tomography (OCT) (see, Kanai et al., 2018, American Journal of Ophthalmology Case Reports, 10:137-141, incorporated herein by reference in its entirety). Visual function can be assessed, for example, by measuring a subject's contrast sensitivity using a standard test chart (e.g., CSV-1000E chart; Vector Vision Co., Greenville, OH). Straylight measurements can be used to quantify light scattering that results in a veil of straylight over the retinal image, which can lead to hazy vision or increased glare hindrance. Straylight can be measured using a straylight meter (e.g., C-Quant from Oculus GmbH, Wetzlar, Germany). In certain embodiments, methods of the disclosure can reduce the severity of a subject's fish eye disease, for example as measured by corneal opacity, contrast sensitivity, straylight values, or a combination thereof.

In some embodiments the subject does not have an LCAT deficiency.

In some embodiments, the subject has an eye disease that is other than fish-eye disease, e.g., an eye disease described herein other than fish-eye disease, and optionally wherein the lipid binding protein-based complex used to treat the subject is CER-001.

In some embodiments, the subject has a genetic disease such as Stargardt disease, optionally wherein the lipid binding protein-based complex used to treat the subject is CER-001.

In some embodiments, the subject has macular degeneration, e.g., dry AMD or wet AMD, optionally wherein the lipid binding protein-based complex used to treat the subject is CER-001. In other embodiments, the subject has an eye disease which is other than macular degeneration, e.g., an eye disease described herein other than macular degeneration.

In some embodiments, the subject has diabetic retinopathy, optionally wherein the lipid binding protein-based complex used to treat the subject is CER-001. In some embodiments, a subject with diabetic retinopathy has diabetic macular edema.

In some embodiments, the subject has retinal vein occlusion.

In some embodiments, the subject has dry eye disease (e.g., severe dry eye disease). In some embodiments, the subject has Meibomian gland dysfunction (MBD), for example obstructive MGD. In other embodiments, the subject has lacrimal gland dysfunction. In some embodiments, the subject has blepharitis. In some embodiments, the subject has uveitis (e.g., caused by a bacterial infection). In some embodiments, the subject has lipid keratopathy. In some embodiments, the lipid binding protein-based complex used to treat the subject having one of the eye diseases described in this paragraph is CER-001.

In some embodiments, the subject has ocular lipid deposits comprising lipid deposits present in the eye and/or near the eye. In one embodiment, the lipid deposits are corneal lipid deposits, retinal lipid deposits, palpebral lipid deposits or a combination thereof. In some embodiments, the lipid binding protein-based complex used to treat the subject having one of the eye diseases described in this paragraph is CER-001.

In some embodiments, the subject has lipid deposits in the cornea and/or in the retina. Lipid deposits in the cornea can cause vision impairment, e.g., blurred vision. Lipids deposits in the retina, such as Drusen in dry AMD or lipofuscins in Stargardt disease, can lead to degeneration of the retina. In some embodiments, the lipid binding protein-based complex used to treat the subject having one of the eye diseases described in this paragraph is CER-001.

In some embodiments, the subject has palpebral lipid deposits, which are lipid deposits on the eyelids.

In some embodiments, the lipid deposits are within Drusen deposits. Drusen are focal deposits of extracellular debris located between the basal lamina of the retinal pigment epithelium (RPE) and the inner collagenous layer of Bruch's membrane. Most drusen are of the hard type, which can be dome shaped with solid interiors and homogeneous contents, and a median diameter of 47 μm. Hard drusen comprise lipid particles of about 60-90 nm in diameter containing abundant esterified cholesterol, unesterified cholesterol, phosphatidylcholine, and apolipoprotein B. The presence of a few hard drusen is normal with advancing age. The presence of larger and more numerous drusen in the macula is a common early sign of age-related macular degeneration (AMD).

In one embodiment, the lipid deposits are lipofuscin granules. Lipofuscin granules accumulate in postmitotic RPE lysosomal compartments. Lipofuscin granules mainly comprise N-retinylidene-N-retinyl-ethanolamine (A2E). The presence of lipofuscin granules is a sign of Stargardt disease.

In one embodiment, the lipid deposits are cholesterol depots, especially cholesterol depots in the cornea. In individuals with genetic deficiency of LCAT, cholesterol accumulates within the extracellular connective tissue matrix of the cornea stroma. Usually, such cholesterol depots have 0.2 to 2.5 μm in diameter.

In one embodiment, the ocular lipid deposits are not calcified.

The presence of ocular lipid deposits can be determined by one or more of slit lamp photography of the retina, Heidelberg retina tomograph (HRT) scan, optical coherence tomography (OCT), fundus autofluorescence imaging, eye fundus by slit lamp. Especially, Drusen can be observed by slit lamp photography of the retina, HRT scan and/or OCT; lipofuscin granules can be observed by fundus autofluorescence imaging; and cholesterol depots in the cornea can be observed by slit lamp.

The use of the lipid binding protein complexes described herein can reduce the severity of a subject's eye disease. Without willing bound by a theory, it is thought that the lipid binding protein complexes can solubilize lipids accumulated in ocular deposits, leading to their elimination.

In some embodiments, use of the lipid binding protein complexes described herein can reduce the number of ocular lipid deposits. In some embodiments, use of the lipid binding protein complexes described herein can reduce the size of the ocular lipid deposits.

The reduction in number and/or in size of the lipid deposits can be qualitatively evaluated, for example by comparing the results of exams performed before the administration of a lipid binding protein complex and during or after administration, such as slit lamp photography of the retina, Heidelberg retina tomograph (HRT) scan, optical coherence tomography (OCT), fundus autofluorescence imaging, eye fundus by slit lamp. The reduction in number and/or in size of the lipid deposits can also be quantitatively evaluated by above methods.

Alternatively, the reduction in number and/or in size of the lipid deposits can be indirectly determined by comparing the results measures of the corneal opacity, contrast sensitivity, straylight values, or a combination thereof, obtained before the administration of the lipoprotein complex and during or after administration.

In certain embodiments, the reduction of the severity of the eye disease can for example be measured by evaluation of corneal opacity, contrast sensitivity, straylight values, or a combination thereof.

In one embodiment, the subject has impaired vision, including blurred vision, due to ocular lipid deposits and the amount of the lipoprotein complex is an amount which improves the subject's vision.

In one embodiment, the subject has corneal opacity due to lipid deposits in the cornea. Treatment with a lipid binding protein complex described herein can reduce the opacity of the subject's cornea(s). Corneal opacity and its progression or regression (e.g., in response to treatment as described herein) can be qualitatively evaluated, for example by comparing slit lamp images of a subject's eyes taken at different times. Corneal opacity can also be evaluated quantitatively, for example by anterior segment optical coherence tomography (OCT) (see, Kanai et al., 2018, American Journal of Ophthalmology Case Reports, 10:137-141, incorporated herein by reference in its entirety). Visual function can be assessed, for example, by measuring a patient's contrast sensitivity using a standard test chart (e.g., CSV-1000E chart; Vector Vision Co., Greenville, OH). Straylight measurements can be used to quantify light scattering that results in a veil of straylight over the retinal image, which can lead to hazy vision or increased glare hindrance. Straylight can be measured using a straylight meter (e.g., C-Quant from Oculus GmbH, Wetzlar, Germany).

In one embodiment, the amount of the lipid binding protein complex administered is an amount effective to reduce the opacity of the patient's cornea(s).

In one embodiment, the amount of the lipid binding protein complex administered is effective to improve the patient's contrast sensitivity.

In one embodiment, the amount of the lipid binding protein complex administered is effective to reduce the patient's straylight values.

In other aspects, the subject has an eye disease (which can be, but is not necessarily a disease associated with lipid accumulation) and a lipid-binding protein-based complex is used as a drug carrier to deliver one or more ophthalmic drugs to the eye of the subject. For example, the subject can have an anterior ocular condition or a posterior ocular condition, for example uveitis (e.g., caused by a bacterial infection), macular edema (e.g., diabetic macular edema), macular degeneration, retinal detachment, an ocular tumor, a fungal infection, a viral infection, a bacterial infection such as bacterial conjunctivitis or trachoma, multifocal choroiditis, diabetic retinopathy, proliferative vitreoretinopathy (PVR), sympathetic ophthalmia, Vogt Koyanagi-Harada (VKH) syndrome, histoplasmosis, uveal diffusion, vascular occlusion, endophthalmitis, or glaucoma.

### 6.2.1. CER-001 for Use in Treating Uveitis

Inflammatory eye disease such as uveitis (e.g., anterior uveitis, intermediate uveitis, posterior uveitis, or panuveitis), which may or may not be caused by bacterial infection, can treated by the administration of CER-001. The use of CER-001 to treat uveitis can be accomplished by administering a therapeutically effective amount of CER-001 to a subject in need thereof, e.g., an amount which reduces the severity of the uveitis (e.g., by alleviating one or more symptoms of the uveitis). CER-001 can be suitably formulated for the intended route of administration. Exemplary formulations are described in Section 6.1.10. For the treatment of uveitis, local administration of CER-001 such as topical administration or intraocular administration to a subject in need thereof is preferred. Intraocular administration can be by, for example, intraocular injection, for example intra-vitreal injection, sub-conjunctival injection, parabulbar injection, peribulbar injection retro-bulbar injection, suprachoroidal injection or suprascleral injection. Implants can also be used to deliver CER-001. For topical administration, CER-001 can be administered, for example, as an eye drop. As shown in Examples 3 and 4, good ocular tolerance of CER-001 was observed even with repeated administrations.

In some embodiments, CER-001 can be used for the treatment of uveitis in a subject in need thereof in accordance with the dosage regimen described in one or more of Sections 6.3, 6.4, and 6.5. For example, CER-001 can be used for the treatment of uveitis in a subject in need thereof in accordance with an induction regimen described in Section 6.3. Alternatively, or in addition, CER-001 can be used for the treatment of uveitis in a subject in need thereof in accordance with a consolidation regimen described in Section 6.4. Alternatively, or in addition, CER-001 can be used for the treatment of uveitis in a subject in need thereof in accordance with a maintenance regimen described in Section 6.5. In some embodiments, CER-001 can be used for the treatment of uveitis in a subject in need thereof in accordance with an induction regimen described in Section 6.3; and/or a consolidation regimen described in Section 6.4; and/or a maintenance regimen described in Section 6.5.

6.2.2. CER-001 with an Ophthalmic Drug for Use in Treating Eye Diseases

In some embodiments, inflammatory eye disease such as uveitis (e.g., anterior uveitis (for example acute anterior uveitis), intermediate uveitis, posterior uveitis, or panuveitis), which may or may not be caused by bacterial infection, can treated by the administration of a composition comprising CER-001 and dexamethasone, a composition comprising CER-001 and dexamethasone palmitate, or a composition comprising CER-001 and tacrolimus. The use of such compositions to treat uveitis can be accomplished by administering a therapeutically effective amount of the composition to a subject in need thereof, e.g., an amount which reduces the severity of the uveitis (e.g., by alleviating one or more symptoms of the uveitis). The composition can be suitably formulated for the intended route of administration. Exemplary formulations are described in Section 6.1.10. For the treatment of uveitis, local administration of the composition such as topical administration or intraocular administration to a subject in need thereof is preferred. Intraocular administration can be by, for example, intraocular injection, for example intra-vitreal injection, sub-conjunctival injection, parabulbar injection, peribulbar injection retro-bulbar injection, suprachoroidal injection or suprascleral injection. Implants can also be used. For topical administration, the composition can be administered, for example, as an eye drop. As shown in Examples 3 and 4, good ocular tolerance of such compositions was observed even with repeated administrations.

In some embodiments, a composition comprising CER-001 and dexamethasone, a composition comprising CER-001 and dexamethasone palmitate, or a composition comprising CER-001 and tacrolimus can be used for the treatment of uveitis in a subject in need thereof in accordance with the dosage regimen described in one or more of Sections 6.3, 6.4, and 6.5. For example, the composition can be used for the treatment of uveitis in a subject in need thereof in accordance with a induction regimen described in Section 6.3. Alternatively, or in addition, the composition can be used for the treatment of uveitis in a subject in need thereof in accordance with a consolidation regimen described in Section 6.4. Alternatively, or in addition, the composition can be used for the treatment of uveitis in a subject in need thereof in accordance with a maintenance regimen described in Section 6.5. In some embodiments, the composition can be used for the treatment of uveitis in a subject in need thereof in accordance with an induction regimen described in Section 6.3; and/or a consolidation regimen described in Section 6.4; and/or a maintenance regimen described in Section 6.5.

Compositions comprising CER-001 and dexamethasone and compositions comprising CER-001 and dexamethasone palmitate can also be used to treat other eye diseases such as macular degeneration and dry macular edema (e.g., by alleviating one or more symptoms of the disease). Compositions comprising CER-001 and tacrolimus can also be used to treat other eye diseases, for example dry eye disease, e.g., severe dry eye disease (e.g., by alleviating one or more symptoms of the disease).

Compositions comprising CER-001 and dexamethasone, compositions comprising CER-001 and dexamethasone palmitate, and compositions comprising CER-001 and tacrolimus can in some embodiments be made by thermal cycling a mixture comprising CER-001 and the respective drug, for example as described in Section 6.1.10.

### 6.3. Induction Regimen

Induction regimens suitable for use in the methods of the disclosure entail administering multiple doses of a lipid binding protein-based complex (e.g., CER-001) separated by 1 or more day between each administration.

The induction regimens typically include at least three doses of a lipid binding protein-based complex (e.g., CER-001) but can include four or more doses of a lipid binding protein-based complex (e.g., CER-001), e.g., five, six, seven, eight, nine, ten, eleven or twelve doses.

The induction regimens can last one or more weeks, two or more weeks, three or more weeks, four or more weeks, five or more weeks, six or more weeks, seven or more weeks, eight or more weeks, nine or more weeks, or ten or more weeks.

For example, the induction regimen can comprise administering:
three doses of a lipid binding protein-based complex (e.g., CER-001) over one week;
three doses of a lipid binding protein-based complex (e.g., CER-001) over two weeks;
three doses of a lipid binding protein-based complex (e.g., CER-001) over three weeks;
four doses of a lipid binding protein-based complex (e.g., CER-001) over two weeks;
four doses of a lipid binding protein-based complex (e.g., CER-001) over three weeks;
five doses of a lipid binding protein-based complex (e.g., CER-001) over two weeks;
five doses of a lipid binding protein-based complex (e.g., CER-001) over three weeks;
five doses of a lipid binding protein-based complex (e.g., CER-001) over four weeks;
six doses of a lipid binding protein-based complex (e.g., CER-001) over two weeks;
six doses of a lipid binding protein-based complex (e.g., CER-001) over three weeks;
six doses of a lipid binding protein-based complex (e.g., CER-001) over four weeks;
seven doses of a lipid binding protein-based complex (e.g., CER-001) over three weeks;
seven doses of a lipid binding protein-based complex (e.g., CER-001) over four weeks;
seven doses of a lipid binding protein-based complex (e.g., CER-001) over five weeks;
eight doses of a lipid binding protein-based complex (e.g., CER-001) over three weeks;
eight doses of a lipid binding protein-based complex (e.g., CER-001) over four weeks;
eight doses of a lipid binding protein-based complex (e.g., CER-001) over five weeks;
nine doses of a lipid binding protein-based complex (e.g., CER-001) over three weeks;
nine doses of a lipid binding protein-based complex (e.g., CER-001) over four weeks;
nine doses of a lipid binding protein-based complex (e.g., CER-001) over five weeks;
nine doses of a lipid binding protein-based complex (e.g., CER-001) over six weeks;
ten doses of a lipid binding protein-based complex (e.g., CER-001) over four weeks;
ten doses of a lipid binding protein-based complex (e.g., CER-001) over five weeks;
ten doses of a lipid binding protein-based complex (e.g., CER-001) over six weeks; or
ten doses of a lipid binding protein-based complex (e.g., CER-001) over seven weeks.

In an embodiment, the induction regimen comprises two doses of a lipid binding protein-based complex (e.g., CER-001) per week to five doses per week.

In an embodiment, the induction regimen comprises administering nine doses of a lipid binding protein-based complex (e.g., CER-001) over three weeks, e.g., on days 1, 2, 4, 7, 9, 11, 14, 16, and 18.

In practice, an administration window can be provided, for example, to accommodate slight variations to a multi-dosing per week dosing schedule. For example, a window of ±2 days or ±1 day around the dosage date can be used.

A therapeutic dose of a lipid binding protein-based complex (e.g., CER-001) administered by infusion in the induction regimen can range from 4 to 30 mg/kg on a protein weight basis (e.g., 4, 5, 6, 7, 8, 9, 10, 12 15, 20, 25, or 30 mg/kg, or any range bounded by any two of the foregoing values, e.g., 5 to 15 mg/kg, 10 to 20 mg/kg, or 15 to 25 mg/kg). As used herein, the expression “protein weight basis” means that a dose of a lipid binding protein-based complex (e.g., CER-001) to be administered to a subject is calculated based upon the amount of lipid binding protein (e.g., ApoA-I) in the a lipid binding protein-based complex (e.g., CER-001) to be administered and the weight of the subject. For example, a subject who weighs 70 kg and is to receive a 10 mg/kg dose of CER-001 would receive an amount of CER-001 that provides 700 mg of ApoA-I (70 kg×10 mg/kg). In some embodiments, the dose of a lipid binding protein-based complex (e.g., CER-001) used in the induction regimen is 8 mg/kg. In some embodiments, the induction regimen comprises nine doses of a lipid binding protein-based complex (e.g., CER-001) administered over three weeks at a dose of 8 mg/kg. In some embodiments, the dose of a lipid binding protein-based complex (e.g., CER-001) used in the induction regimen is 10 mg/kg. In some embodiments, the dose of a lipid binding protein-based complex (e.g., CER-001) used in the induction regimen is 15 mg/kg. In some embodiments, the dose of a lipid binding protein-based complex (e.g., CER-001) used in the induction regimen is 20 mg/kg. In some embodiments, the induction regimen comprises nine doses of a lipid binding protein-based complex (e.g., CER-001) administered over three weeks at a dose of 10 mg/kg.

The dose of a lipid binding protein-based complex used to deliver an ophthalmic drug can be a dose that delivers a therapeutically effective amount of the drug.

In yet other aspects, a lipid binding protein-based complex (e.g., CER-001) can be administered on a unit dosage basis. The unit dosage used in the induction phase can vary from 300 mg to 3000 mg per administration by infusion.

In particular embodiments, the dosage of a lipid binding protein-based complex (e.g., CER-001) used during the induction phase is 300 mg to 1500 mg, 400 mg to 1500 mg, 500 mg to 1200 mg, or 500 mg to 1000 mg per administration by infusion.

In particular embodiments, a lipid binding protein-based complex (e.g., CER-001) is administered as an IV infusion. For example, a stock solution of CER-001 can be diluted in normal saline such as physiological saline (0.9% NaCl) to a total volume between 125 and 250 ml. In a preferred embodiment, subjects weighing less than 80 kg will have a total volume of 125 ml whereas subjects weighing at least 80 kg will have a total volume of 250 ml. A lipid binding protein-based complex (e.g., CER-001) may be administered over a one-hour period using an infusion pump at a fixed rate of 250 ml/hr. Depending on the needs of the subject, administration can be by slow infusion with a duration of more than one hour (e.g., up to two hours), by rapid infusion of one hour or less, or by a single bolus injection.

In alternative embodiments, a lipid binding protein-based complex (e.g., CER-001) is administered locally to the eye, for example, by intraocular injection or topical administration. A stock solution of a lipid binding protein-based complex (e.g., CER-001) can be diluted in a suitable diluent prior to administration. Suitable diluents include normal saline such as physiological saline (0.9% NaCl). In some embodiments, the lipid binding protein-based complex is formulated as an eye drop.

### 6.4. Consolidation Regimen

Consolidation regimens suitable for use in the methods of the disclosure entail administering multiple doses of a lipid binding protein-based complex (e.g., CER-001) separated by 1 day or greater between each dose e.g., 2 days for greater between each administration.

The consolidation regimens typically include at least two doses of a lipid binding protein-based complex (e.g., CER-001) but can include three or more doses of a lipid binding protein-based complex (e.g., CER-001), e.g., four, five, six, seven, eight, nine or ten.

The consolidation regimens can last one or more weeks, two or more weeks, three or more weeks, four or more weeks, five or more weeks, six or more weeks, seven or more weeks, eight or more weeks, nine or more weeks, or ten or more weeks.

For example, the consolidation regimen can comprise administering:
two doses of a lipid binding protein-based complex (e.g., CER-001) over one week;
two doses of a lipid binding protein-based complex (e.g., CER-001) over two weeks;
three doses of a lipid binding protein-based complex (e.g., CER-001) over two weeks;
three doses of a lipid binding protein-based complex (e.g., CER-001) over three weeks;
four doses of a lipid binding protein-based complex (e.g., CER-001) over two weeks;
four doses of a lipid binding protein-based complex (e.g., CER-001) over three weeks;
five doses of a lipid binding protein-based complex (e.g., CER-001) over three weeks;
five doses of a lipid binding protein-based complex (e.g., CER-001) over four weeks;
five doses of a lipid binding protein-based complex (e.g., CER-001) over five weeks;
six doses of a lipid binding protein-based complex (e.g., CER-001) over three weeks;
six doses of a lipid binding protein-based complex (e.g., CER-001) over four weeks;
six doses of a lipid binding protein-based complex (e.g., CER-001) over five weeks;
seven doses of a lipid binding protein-based complex (e.g., CER-001) over four weeks;
seven doses of a lipid binding protein-based complex (e.g., CER-001) over five weeks;
seven doses of a lipid binding protein-based complex (e.g., CER-001) over six weeks;
eight doses of a lipid binding protein-based complex (e.g., CER-001) over four weeks;
eight doses of a lipid binding protein-based complex (e.g., CER-001) over five weeks;
eight doses of a lipid binding protein-based complex (e.g., CER-001) over six weeks;
nine doses of a lipid binding protein-based complex (e.g., CER-001) over four weeks;
nine doses of a lipid binding protein-based complex (e.g., CER-001) over five weeks;
nine doses of a lipid binding protein-based complex (e.g., CER-001) over six weeks;
nine doses of a lipid binding protein-based complex (e.g., CER-001) over six weeks;
ten doses of a lipid binding protein-based complex (e.g., CER-001) over four weeks;
ten doses of a lipid binding protein-based complex (e.g., CER-001) over five weeks;
ten doses of a lipid binding protein-based complex (e.g., CER-001) over six weeks; or
ten doses of a lipid binding protein-based complex (e.g., CER-001) over seven weeks.

In an embodiment, the consolidation regimen comprises two doses of a lipid binding protein-based complex (e.g., CER-001) per week to five doses per week.

In an embodiment, the consolidation regimen comprises administering six doses of a lipid binding protein-based complex (e.g., CER-001) over three weeks, e.g., on days 21, 24, 28, 31, 35 and 38 of a treatment regimen that begins with an induction regimen on day 1.

In practice, an administration window can be provided, for example, to accommodate slight variations to a multi-dosing per week dosing schedule. For example, a window of ±2 days or ±1 day around the dosage date can be used.

A therapeutic dose of a lipid binding protein-based complex (e.g., CER-001) administered by infusion in the consolidation regimen can range from 4 to 30 mg/kg on a protein weight basis (e.g., 4, 5, 6, 7, 8, 9, 10, 12, 15, 20, 25, or 30 mg/kg, or any range bounded by any two of the foregoing values, e.g., 5 to 15 mg/kg, 10 to 20 mg/kg, or 15 to 25 mg/kg). In some embodiments, the dose of a lipid binding protein-based complex (e.g., CER-001) used in the consolidation regimen is 8 mg/kg. In some embodiments, the consolidation regimen comprises six doses of a lipid binding protein-based complex (e.g., CER-001) administered over three weeks at a dose of 8 mg/kg. In some embodiments, the dose of a lipid binding protein-based complex (e.g., CER-001) used in the consolidation regimen is 10 mg/kg. In some embodiments, the dose of a lipid binding protein-based complex (e.g., CER-001) in the consolidation regimen is 15 mg/kg. In some embodiments, the dose of a lipid binding protein-based complex (e.g., CER-001) used in the consolidation regimen is 20 mg/kg. In some embodiments, the consolidation regimen comprises six doses of a lipid binding protein-based complex (e.g., CER-001) administered over three weeks at a dose of 10 mg/kg. The dose of a lipid binding protein-based complex used to deliver an ophthalmic drug can be a dose that delivers a therapeutically effective amount of the drug.

In yet other aspects, a lipid binding protein-based complex (e.g., CER-001) can be administered on a unit dosage basis. The unit dosage used in the consolidation phase can vary from 300 mg to 3000 mg per administration by infusion.

In particular embodiments, the dosage of a lipid binding protein-based complex (e.g., CER-001) used during the consolidation phase is 300 mg to 1500 mg, 400 mg to 1500 mg, 500 mg to 1200 mg, or 500 mg to 1000 mg per administration by infusion.

In some embodiments, the dose of the a lipid binding protein-based complex (e.g., CER-001) administered during the consolidation phase is greater than the dose of the a lipid binding protein-based complex (e.g., CER-001) administered during the induction phase. For example, the dose administered in the consolidation phase can be 1.5 to 3 times the dose administered in the induction phase. In specific embodiments, the dose of a lipid binding protein-based complex (e.g., CER-001) administered in the consolidation phase is 2 times the dose of the lipid binding protein-based complex (e.g., CER-001) administered in the consolidation phase. Increasing the dose in the consolidation phase can offset the reduced frequency of dosing. In other embodiments, the dose of the lipid binding protein-based complex (e.g., CER-001) administered during the consolidation phase is the same as the dose of the lipid binding protein-based complex (e.g., CER-001) administered during the induction phase.

The lipid binding protein-based complex (e.g., CER-001) can be administered during the consolidation phase in the same manner as described in Section 6.3, e.g., as an IV infusion over a one-hour period or administered locally such as intraocular or topical administration. When the dose of lipid binding protein-based complex (e.g., CER-001) administered during the consolidation phase is larger than the dose administered in the induction phase, the lipid binding protein-based complex (e.g., CER-001) can optionally be administered in a larger volume and/or infused and/or administered over a longer period of time. For example, when the dose of the lipid binding protein-based complex (e.g., CER-001) administered during the consolidation phase is twice the dose administered during the induction phase, the administration volume can be increased (e.g., doubled) and/or the infusion time can be increased (e.g., doubled).

### 6.5. Maintenance Regimen

The methods of the disclosure can comprise a maintenance regimen, which can, but does not necessarily follow an induction regimen and optionally a consolidation regimen. In some embodiments, a maintenance regimen comprises administering a lipid binding protein-based complex (e.g., CER-001) to a subject on a less frequent basis than during the induction phase and/or the consolidation phase. Typically, the lipid binding protein-based complex (e.g., CER-001) is administered once every 3 or more days, for example once every week or twice a week, during the maintenance regimen.

The maintenance regimen can entail administering the lipid binding protein-based complex (e.g., CER-001) for one month or longer, two months or longer, three months or longer, six months or longer, nine months or longer, a year or longer, 18 months or longer, two years or longer, or indefinitely.

In some embodiments, the maintenance regimen comprises administering a lipid binding protein-based complex (e.g., CER-001) once every 5 days to one week for at least 16 weeks. In other embodiments, the maintenance regimen comprises administering a lipid binding protein-based complex (e.g., CER-001) once every week for at least 20 weeks, for at least 30 weeks, or for at least 40 weeks.

Similar to the administration window described above in Section 6.3, an administration window can also be used in the maintenance regimen to accommodate slight variations to a weekly dosing schedule. For example, a window of +2 days or +1 day around the weekly date can be used.

A therapeutic dose of a lipid binding protein-based complex (e.g., CER-001) administered by infusion in the maintenance regimen can range from 4 to 30 mg/kg on a protein weight basis (e.g., 4, 5, 6, 7, 8, 9, 10, 12, 15, 20, 25, or 30 mg/kg, or any range bounded by any two of the foregoing values, e.g., 5 to 15 mg/kg, 10 to 20 mg/kg, or 15 to 25 mg/kg). For example, a subject who weighs 70 kg and is to receive a 10 mg/kg dose of CER-001 would receive an amount of CER-001 that provides 700 mg of ApoA-I (70 kg×10 mg/kg). In some embodiments, the dose of lipid binding protein-based complex (e.g., CER-001) used in the maintenance regimen is 8 mg/kg. In some embodiments, the dose of lipid binding protein-based complex (e.g., CER-001) used in the maintenance regimen is 10 mg/kg. In some embodiments, the dose of lipid binding protein-based complex (e.g., CER-001) used in the consolidation regimen is 15 mg/kg. In some embodiments, the dose of lipid binding protein-based complex (e.g., CER-001) used in the consolidation regimen is 20 mg/kg. The dose of a lipid binding protein-based complex used to deliver an ophthalmic drug can be a dose that delivers a therapeutically effective amount of the drug.

In yet other aspects, a lipid binding protein-based complex (e.g., CER-001) can be administered on a unit dosage basis. The unit dosage used in the maintenance phase can vary from 300 mg to 3000 mg per administration by infusion.

In particular embodiments, the dosage of a lipid binding protein-based complex (e.g., CER-001) used during the maintenance phase is 300 mg to 1500 mg, 400 mg to 1500 mg, 500 mg to 1200 mg, or 500 mg to 1000 mg per administration by infusion.

In some embodiments, the dose of the lipid binding protein-based complex (e.g., CER-001) administered during the maintenance phase is greater than the dose of the lipid binding protein-based complex (e.g., CER-001) administered during the induction phase and/or consolidation phase. For example, the dose administered in the maintenance phase can be 1.5 to 3 times the dose administered in the consolidation phase. In specific embodiments, the dose of lipid binding protein-based complex (e.g., CER-001) administered in the maintenance phase is 2 times the dose of the lipid binding protein-based complex (e.g., CER-001) administered in the consolidation phase. Increasing the dose in the maintenance phase can offset the reduced frequency of dosing. In other embodiments, the dose of the lipid binding protein-based complex (e.g., CER-001) administered during the maintenance phase is the same as the dose of the lipid binding protein-based complex (e.g., CER-001) administered during the induction phase and/or consolidation phase. In some embodiments, the dose administered in the maintenance phase can be adjusted, for example increased or decreased, for example to reach dose that stabilizes a clinical parameter (e.g., corneal opacity). Alternatively or in addition, the administration frequency in the maintenance phase can be adjusted, for example increasing or decreasing the frequency, for example to achieve stabilization of a clinical parameter (e.g., corneal opacity).

A lipid binding protein-based complex (e.g., CER-001) can be administered during the maintenance phase in the same manner as described in Section 6.3, e.g., as an IV infusion or administered locally such as introcular or topical administration. When the dose of lipid binding protein-based complex (e.g., CER-001) administered during the maintenance phase is larger than the dose administered in the consolidation phase, the lipid binding protein-based complex (e.g., CER-001) can optionally be administered in a larger volume and/or infused and/or administered over a longer period of time. For example, when the dose of lipid binding protein-based complex (e.g., CER-001) administered during the maintenance phase is twice the dose administered during the consolidation phase, the administration volume can be increased (e.g., doubled) and/or the infusion time can be increased (e.g., doubled).

### 6.6. Combination Therapies

The subjects can be treated with a lipid binding protein-based complex (e.g., CER-001) as a monotherapy or a part of a combination therapy regimen, e.g., with one or more lipid control medications such as a statin (e.g., atorvastatin, rosuvastatin, simvastatin, fluvastatin, lovastatin, pravastatin), a cholesterol absorption inhibitor (e.g., ezetimibe), niacin, aspirin, a proprotein convertase subtilisin/kexin type 9 (PCSK9) inhibitor (e.g., an antibody such as alirocumab, bococizumab, evolocumab, 1D05-IgG2 (Ni et al., 2011, J Lipid Res. 52 (1): 78-86), and LY3015014 (Kastelein et al., 2016, Eur Heart J 37 (17): 1360-9) or an RNAi therapeutic such as ALN-PCSSC (the Medicines Company)) or an antihypertensive medication (e.g., amlodipine, urapidil, furosemide, and combinations thereof). For example, a subject with fish-eye disease can be treated with one or more lipid control medications in combination with a lipid binding protein-based complex. In some embodiments, a combination therapy comprises a lipid binding protein-based complex (e.g., CER-001) in combination with a standard of care treatment for the subject's eye disease.

A combination therapy regimen can entail administering a lipid binding protein-based complex (e.g., CER-001) in combination with one or more of the foregoing medicines and/or one or more of the foregoing classes of medications. In some embodiments, the subject is treated with a lipid binding protein-based complex (e.g., CER-001) in combination with atorvastatin. In some embodiments, the subject is treated with a lipid binding protein-based complex (e.g., CER-001) in combination with ezetimibe. In some embodiments, the subject is treated with a lipid binding protein-based complex (e.g., CER-001) in combination with niacin. In some embodiments, the subject is treated with a lipid binding protein-based complex (e.g., CER-001) in combination with rosuvastatin. In some embodiments, the subject is treated with a lipid binding protein-based complex (e.g., CER-001) in combination with simvastatin. In some embodiments, the subject is treated with a lipid binding protein-based complex (e.g., CER-001) in combination with aspirin. In some embodiments, the subject is treated with a lipid binding protein-based complex (e.g., CER-001) in combination with fluvastatin. In some embodiments, the subject is treated with a lipid binding protein-based complex (e.g., CER-001) in combination with lovastatin. In some embodiments, the subject is treated with a lipid binding protein-based complex (e.g., CER-001) in combination with pravastatin. In some embodiments, the subject is treated with a lipid binding protein-based complex (e.g., CER-001) in combination with alirocumab. In some embodiments, the subject is treated with a lipid binding protein-based complex (e.g., CER-001) in combination with evolocumab. In some embodiments, the subject is treated with a lipid binding protein-based complex (e.g., CER-001) in combination with ALN-PCSsc. In each of the foregoing embodiments, the lipid control medicine can be the only lipid control medicine that the subject receives in combination with lipid binding protein-based complex therapy, or can be part of a combination of lipid control medicines administered in combination with the lipid binding protein-based complex.

In some embodiments, a lipid binding protein-based complex (e.g., CER-001) is administered in combination with an antihypertensive medication, e.g., one, two, or all three of amlodipine, urapidil, and furosemide. In some embodiments, a lipid binding protein-based complex (e.g., CER-001) is administered in combination with amlodipine, urapidil, and furosemide. In some embodiments, the combination further comprises a statin, e.g., atorvastatin.

Therapy with a lipid binding protein-based complex (e.g., CER-001) can be added to a background lipid lowering therapy started before therapy with a lipid binding protein-based complex (e.g., CER-001).

In some embodiments, the subject is treated with a stable dose of a lipid control medication for at least 6 weeks (e.g., 6 weeks, 8 weeks, 2 months, 6 months, 1 year, or more than one year) before beginning therapy with a lipid binding protein-based complex (e.g., CER-001) according to a dosing regimen of the disclosure. Alternatively, a lipid binding protein-based complex (e.g., CER-001) therapy can be started before or concurrently with treatment with one or more lipid control medications.

### 7. EXAMPLES

### 7.1. Example 1: CER-001 as a Carrier for Ophthalmic Drugs

### 7.1.1. Azithromycin and Spironolactone

Azithromycin is an antibiotic used to treat bacterial infections of the eye such as bacterial conjunctivitis and trachoma (www.mayoclinic.org/drugs-supplements/azithromycin-ophthalmic-route/description/drg-20070979). Spironolactone is steroid which has been investigated for the treatment of meibomian gland dysfunction and associated dry eye (Yee et al., 2016, Investigative Ophthalmology & Visual Science 57 (12): 5664). A study was conducted to evaluate the suitability of CER-001 to act as a drug carrier for the delivery of azithromycin and spironolactone.

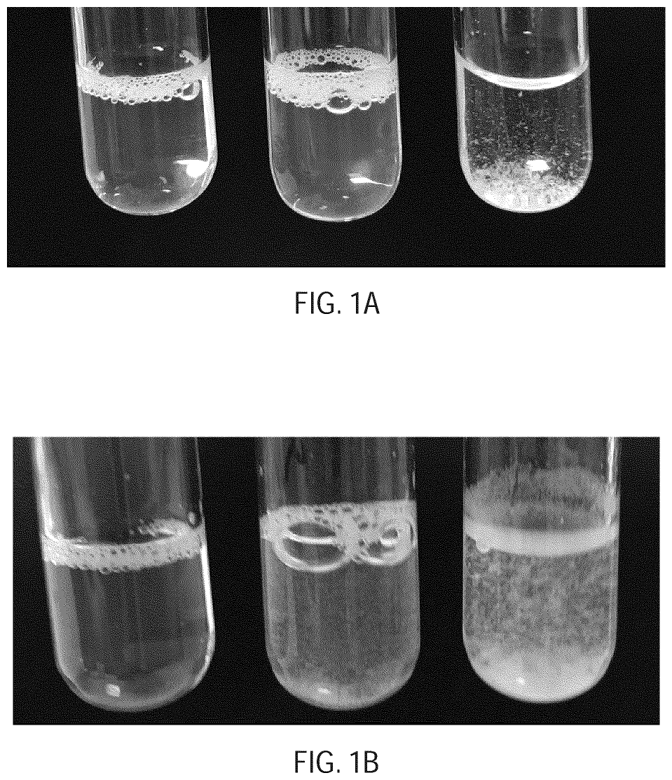

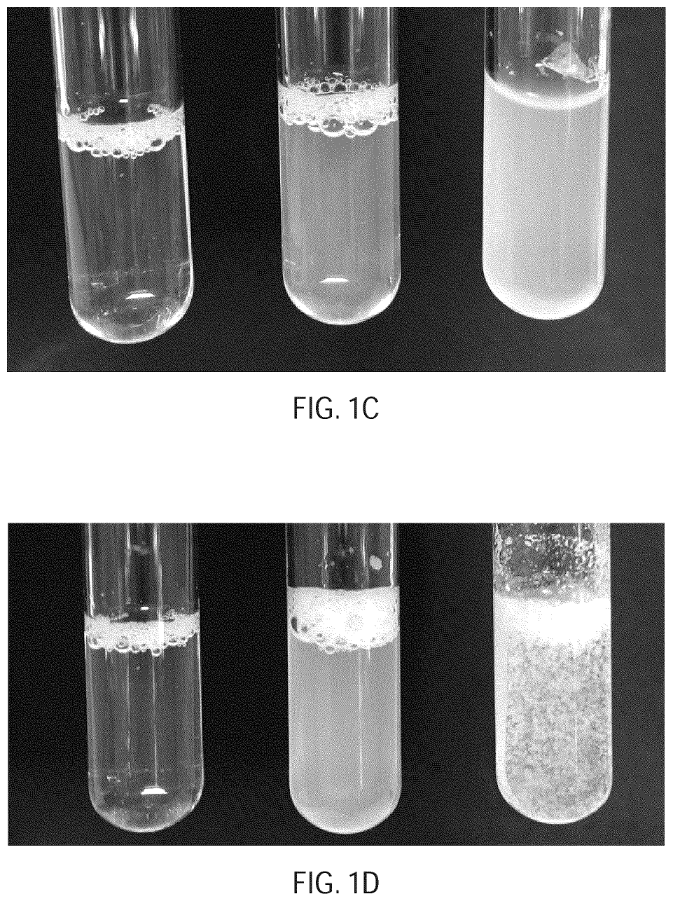

A solution of CER-001 was mixed with azithromycin to provide a final azithromycin concentration of 10 mg/ml and subjected to five heating and cooling cycles from 37° C. to 55° C. to promote complexation of azithromycin to CER-001. For controls, a sample of CER-001 without added azithromycin and a sample of azithromycin at a concentration of 10 mg/ml in phosphate buffered saline (PBS) were similarly subjected to five heating and cooling cycles from 37° C. to 55° C. As shown in FIG. 1A, the sample of CER-001 without azithromycin (left tube) remained clear following the heating and cooling cycles, while the sample of azithromycin in PBS (right tube) contained numerous crystals in the liquid and on the glass tube. The sample of CER-001 with azithromycin (middle tube) was cloudier than the pure CER-001 sample, but contained significantly fewer azithromycin crystals than the sample of azithromycin in PBS.

A solution of CER-001 was mixed with spironolactone to provide a final spironolactone concentration of 1.5 mg/ml and subjected to five heating and cooling cycles from 37° C. to 55° C. to promote complexation of spironolactone to CER-001. For controls, a sample of CER-001 without added spironolactone and a sample of spironolactone at a concentration of 1.5 mg/ml in phosphate buffered saline (PBS) were similarly subjected to five heating and cooling cycles from 37° C. to 55° C. As shown in FIG. 1B, the sample of CER-001 without spironolactone (left tube) remained clear following the heating and cooling cycles, while the sample of spironolactone in PBS (right tube) contained numerous crystals in the liquid, on the glass tube, and above the meniscus. The sample of CER-001 with spironolactone (middle tube) was cloudier than CER-001 alone, but contained significantly fewer spironolactone crystals than the sample of spironolactone in PBS.

### 7.1.2. Dexamethasone Palmitate

Dexamethasone palmitate is a lipophilic prodrug of dexamethasone and can be used to treat macular edema (Daull et al., 2013, J. Ocul Pharmacol Ther. 29 (2): 258-69). A study was conducted to evaluate the suitability of CER-001 to act as a drug carrier for the delivery of dexamethasone palmitate.

A solution of CER-001 was mixed with dexamethasone palmitate to provide a final dexamethasone palmitate concentration of 1 mg/ml and subjected to five heating and cooling cycles from 37° C. to 55° C. to promote complexation of dexamethasone palmitate to CER-001. For controls, a sample of CER-001 without added dexamethasone palmitate and a sample of dexamethasone palmitate at a concentration of 1 mg/ml in phosphate buffered saline (PBS) were similarly subjected to five heating and cooling cycles from 37° C. to 55° C. As shown in FIG. 1C, the sample of CER-001 without dexamethasone palmitate (left tube) remained clear following the heating and cooling cycles, while the sample of dexamethasone palmitate in PBS (right tube) contained a lipid film on the glass above the meniscus and was very cloudy or “milk-like.”The sample of CER-001 with dexamethasone palmitate (middle tube) was cloudier than CER-001 alone, but contained no precipitation or crystals.

### 7.1.3. Cyclosporine

Cyclosporine is an immunomodulator used to increase tear production in subjects with dry eye (Ames and Galor, 2015, Clin Investig (Longd.) 5 (3): 267-285). A study was conducted to evaluate the suitability of CER-001 to act as a drug carrier for the delivery of cyclosporine.

A solution of CER-001 was mixed with cyclosporine to provide a final cyclosporine concentration of 1 mg/ml and subjected to five heating and cooling cycles from 37° C. to 55° C. to promote complexation of cyclosporine to CER-001. For controls, a sample of CER-001 without added cyclosporine and a sample of cyclosporine at a concentration of 1 mg/ml in phosphate buffered saline (PBS) were similarly subjected to five heating and cooling cycles from 37° C. to 55° C. As shown in FIG. 1D, the sample of CER-001 without cyclosporine (left tube) remained clear following the heating and cooling cycles, while the sample of cyclosporine in PBS (right tube) contained crystals in the glass and in the liquid. The sample of CER-001 with cyclosporine (middle tube) was cloudier than CER-001 alone, but contained no precipitation or crystals, even after overnight storage at 4° C.

This example shows that CER-001 can complex with azithromycin, spironolactone, dexamethasone palmitate, and cyclosporine, indicating that CER-001 is a suitable carrier for ophthalmic drugs.

### 7.2. Example 2: CER-001 Ocular Distribution in Rabbits Following Intravenous Administration

A study was performed to evaluate the distribution of CER-001 in the aqueous humor, the vitreous, the retina, and the choroid of both eyes, and in the plasma after multiple intravenous (IV) administrations in albino rabbits. Briefly, each rabbit was administered a single IV injection of 2 mL/kg of CER-001 at 8.1 mg/ml (16.2 mg/kg) on days 1, 3, and 5. The rabbits were administered two IV injections of 1.25 ml/kg of CER-001 at 8.1 mg/mL (10.13 mg/kg per injection) on day 7. Sampling for bioanalyses was carried out at one hour after the last IV administration.

The greatest amount of CER-001 was detected in the plasma (454 μg/mL). Smaller amounts of CER-001 were found in choroid (16.4 μg/g), retina and aqueous humor (2.1 μg/g and 2.7 μg/mL, respectively). CER-001 was not detected in the vitreous at the time of sampling.

The results of this example indicate that IV administration of CER-001 is associated with the localization of CER-001 to ocular structures.

### 7.3. Example 3: CER-001 Ocular Distribution in Rabbits Following Intravitreal Administration

A study was performed to evaluate the distribution of CER-001 in the aqueous humor, the vitreous, the retina, and the choroid of both eyes, and in the plasma after intravitreal (IVT) administration in albino rabbits. Briefly, a single IVT injection of 50 μL of CER-001 at 8.1 mg/mL was administered to the right eyes of six albino rabbits on day 1, followed by a single IVT injection of 50 μL of CER-001 at 8.1 mg/mL administered to the left eyes on day 2 (two animals) or day 3 (remaining four animals). Sampling for bioanalyses was carried out at 24, 48, or 120 hours after the final injection.

CER-001 was detected in all ocular tissues analyzed. The highest amount of CER-001 was found in the vitreous (approximately 140 μg/mL), which was steady up to 144 hours after injection. The amount of CER-001 was relatively lower in aqueous humor (approximately 20 μg/mL), retina (approximately 40 μg/mL), and choroid (approximately 30 μg/g), yet steady at all sampling timepoints. No systemic exposure was observed as no CER-001 was detected in any of the plasma samples. Full results are shown in the Table 2:

TABLE 2
CER-001 levels in ocular tissues and plasma

| | | CER-001 in | CER-001 | | CER-001 | |
|---|---|---|---|---|---|---|
| | Time | vitreous | in retina | CER-001 in | in choroid | |
| | after | (μg/mL) | (μg/mL) | aqueous humor | (μg/g) | CER-001 |
| Eye | injection | (mean, | (mean, | (μg/mL) (mean, | (mean, | in plasma |
| (all n = 2) | (hr) | calculated) | calculated) | calculated) | calculated) | (μg/mL) |
| Right | 24 | 146.0 | 77.3 | 13.3 | 36.2 | 0.0 |
| Left | 48 | 138.5 | 34.0 | 20.9 | 29.6 | 0.0 |
| Right | 72 | 97.4 | 47.6 | 27.2 | 27.6 | 0.0 |
| Left | 96 | 127.9 | 36.8 | 21.3 | 40.5 | 0.0 |
| Right | 120 | 118.2 | 43.2 | 20.7 | 23.6 | 0.0 |
| Left | 144 | 139.5 | 37.6 | 20.7 | 31.7 | 0.0 |

The results of this example indicate that this mode of administration is effective for the localization of CER-001 formulation in the ocular structures that were analyzed, predominantly in the vitreous. The highest levels being found in the vitreous were expected given the IVT administration route. Distribution to all ocular tissues was steady at all time points. No systemic exposure was observed.

### 7.4. Example 4: CER-001 Therapy for LCAT Deficiency-Related Vision Impairment

A subject with LCAT deficiency and having vision impairment related to the subject's LCAT deficiency (said vision impairment being due to ocular lipid deposits) was administered CER-001 according to a treatment regimen comprising an induction regimen, a consolidation regimen, and a maintenance regimen.

Prior to treatment with CER-001, the subject had ocular lipid deposits presenting as white corneal ring opacities. The subject had normal visual acuity but visual blur, especially at night. Slit-lamp examination and optical computerized tomography showed hyperreflective corneal opacification (data not shown). Next-generation sequencing confirmed the subject as compound-heterozygous for two LCAT gene variations, but none in the ABCA1 or APOA1 genes. The first allele, motherly inherited, is an exon-5 (c.605T>C) missense mutation p. (Ile202Thr), previously well-established as familial LCAT deficiency (FLD)-causing in Europe. The paternal allele (c.154+5G>C), was novel and absent from general referral population databases. It alters a highly evolutionary-conserved residue of intron-1 donor splice-site, thereby potentially altering exon-1 mRNA-splicing and creating a cryptic acceptor splice-site at position c.154+15. Consequently, mRNA incorporation of intronic sequences might generate an abnormal/truncated protein, if not abolish LCAT expression.

The induction regimen comprised nine doses of CER-001 administered over three weeks. The dose of CER-001 administered in the induction regimen was 10 mg/kg, calculated based upon the amount of ApoA-I in the CER-001 administered and the weight of the subject.

Following the induction regimen, the subject was administered CER-001 according to a consolidation regimen comprising seven doses of CER-001 administered over four weeks. The dose of CER-001 administered in the induction regimen was 10 mg/kg, calculated based upon the amount of ApoA-I in the CER-001 administered and the weight of the subject.

Following the consolidation regimen, the subject was administered CER-001 according to a maintenance regimen comprising once a week administration of CER-001 for three weeks. The dose of CER-001 administered in the maintenance regimen was 10 mg/kg calculated based upon the amount of ApoA-I in the CER-001 to be administered and the weight of the subject. Thereafter, the dose was increased to 20 mg/kg once weekly for six weeks. The treatment period was 5 months and was followed by a 3 months off-treatment follow-up period.

In the induction, consolidation and maintenance regimens, CER-001 was administered as an IV infusion after premedication with hydroxyzine. A stock solution of CER-001 was diluted in physiological saline (0.9% NaCl) prior to administration, and all doses of CER-001 were administered using an infusion pump over one hour at a fixed rate of 250 ml/hr.

The subject's vision improved during the course of treatment with CER-001. In particular, administration of CER-001 was accompanied by normalization of vision. At the end of the follow-up period, visual blur did not recur.

CER-001 administered by infusion appears to have reached the anterior segment of the subject's eyes, including the cornea, where it exerted a therapeutic effect. Without being bound by theory, it is believed that the observed effect on the subject's vision is due to the ability of CER-001, even when peripherally administered, to mobilize accumulated lipids in and/or around the eye (e.g., directly or indirectly). Additionally, again without being bound by theory, it is believed that the anti-inflammatory properties of CER-001 may have contributed to the observed effect on the subject's vision.

It is further believed, again without being bound by theory, that subjects suffering from other eye diseases, particularly those associated with accumulation of lipids, can similarly benefit from treatment with CER-001 or another lipid binding protein-based complex. Moreover, and again without being bound by theory, it is believed that the ability of CER-001 to reach the anterior segment of the eye when administered peripherally can be leveraged to deliver ophthalmic drugs to the eye (e.g., the anterior segment of the eye).

### 7.5. Example 5: Ocular Tolerance of CER-001 in Rabbits

Ocular tolerance of CER-001 was assessed in albino rabbits. In the first assessment, CER-001 at 8 mg/mL (on a protein weight basis), with or without complexed dexamethasone palmitate, was administered to the eyes of albino rabbits topically or by a single IVT injection. No tolerance issues were observed in repeated topical administration of up to 8 drops or in single intravitreal administration.

In the second assessment, ocular tolerance of CER-001 with or without soft corticoid (S-Cort) was evaluated following either repeated daily 50 μL instillations or a single 50 μL IVT injection to the right eye. As was the case with the first assessment, no tolerance issues of concern were observed in any of the groups tested, indicating that CER-001 as well as CER-001 with S-Cort are macroscopically well-tolerated.

### 7.6. Example 6: CER-001 Treatment of Endotoxin-Induced Uveitis (Severe Inflammation) in Rabbits

A study was performed to assess the ability of CER-001, with or without complexed dexamethasone palmitate (DXP), to treat endotoxin-induced uveitis (severe inflammation) in albino rabbits when administered topically or by single intravitreal injection (IVT). CER-001 vehicle and Solu-Medrol®, an injectable formulation containing the anti-inflammatory glucocorticoid methylprednisolone sodium succinate, were included as controls. Tolerance was assessed by the McDonald-Shadduck scoring system (see, Eaton et al., Journal of Ocular Pharmacology and Therapeutics 33 (10): 718-734) 6 and 24 hours after administration. Cell infiltration and protein content in the aqueous humor were measured 24 hours after administration.

Briefly, 5 groups of 10 animals were used in a first study:
Group 1: vehicle in topical administration in right eye.
Group 2: CER-001 at 8 mg/ml in topical administration (3 times 50 μl) in right eye.
Group 3: vehicle in IVT administration in right eye.
Group 4: CER-001 at 0.8 mg/ml in 50 μl IVT administration in right eye.
Group 5: Solu-Medrol (as positive control) at 20 mg/ml in 250 μl sub-conjunctival administration in right eye.

One Group was added at one month interval from the previous in-life phase.
Group 6: CER-001 at 8 mg/ml in 50 μl IVT administration in right eye.

One other Group was added at one month interval from the previous in-life phase.
Group 7: CER-S Cort at 8 mg/ml in 50 μl IVT administration in right eye. CER-S Cort (Soft Corticoid) was prepared using 8 mg/ml CER-001.

### 7.6.1. Methods

The right eye of each rabbit was examined using a slit lamp.

The observations were scored using modified McDonald-Shadduck's for conjunctiva (redness and swelling), aqueous flare, and iris, as follows:

Modified Scale from McDonald and Shadduck

### Conjunctivae

| Conjunctival Congestion | Score |
|---|---|
| Normal. May appear blanched to reddish pink without perilimbal | 0 |
| injection (except at 12:00 and 6:00 o'clock positions) with | |
| vessels of the palpebral and bulbar conjunctiva easily observed | |
| A flushed, reddish color predominately confined to the palpebral | 1 |
| conjunctiva with some perilimbal injection but primarily | |
| confined to the lower and upper parts of the eye from the 4:00 | |
| to 7:00 and 11:00 to 1:00 o'clock positions | |
| Bright red color of the palpebral conjunctiva with accompanying | 2 |
| perilimbal injection covering at least 75% of the circumference | |
| of the perilimbal region | |
| Dark, beefy red color with congestion of both the bulbar and | 3 |
| the palpebral conjunctiva along with pronounced perilimbal | |
| injection and the presence of petechia on the conjunctiva. | |
| The petechia generally predominate along the nictitating | |
| membrane and the upper palpebral conjunctiva | |

| Conjunctival Swelling | Score |
|---|---|
| Normal or no swelling of the conjunctival tissue | 0 |
| Swelling above normal without eversion of the lids (can be | 1 |
| easily ascertained by noting that the upper and lower eyelids | |
| are positioned as in the normal eye); swelling generally | |
| starts in the lower cul-de-sac near the inner canthus which | |
| needs slit-lamp examination | |
| Swelling with misalignment of the normal approximation of | 2 |
| the upper and lower eyelids; primarily confined to the | |
| upper eyelid so that in the initial stages the | |
| misapproximation of the eyelids begins by partial eversion | |
| of the upper eyelid. In this stage, swelling is confined | |
| generally to the upper eyelid, although it exists in the | |
| lower cul-de-sac (observed best with the slit-lamp) | |
| Swelling definite with partial eversion of the upper and | 3 |
| lower eyelids essentially equivalent. This can be easily | |
| ascertained by looking at the animal head-on and noticing | |
| the positioning of the eyelids; if the eyes margins do | |
| not meet; eversion has occurred | |
| Eversion of the upper eyelid is pronounced with less | 4 |
| pronounced eversion of the lower eyelid. It is difficult | |
| to retract the lids and observe the perilimbal region | |

### Aqueous Flare

The intensity of the Tyndall phenomenon is scored by comparing the normal Tyndall effect observed when the slit-lamp beam passes through the lens with that seen in the anterior chamber. The presence of aqueous flare is presumptive evidence of breakdown of the blood-aqueous barrier.

| | Score |
|---|---|
| Absence of visible light beam light in the anterior chamber | 0 |
| (no Tyndall effect) | |
| The Tyndall effect is barely discernible. The intensity of | 1 |
| the light beam in the anterior chamber is less than the | |
| intensity of the slit beam as it passes through the lens | |
| The Tyndall beam in the anterior chamber is easily | 2 |
| discernible and is equal in intensity to the slit beam | |
| as it passes through the lens | |
| The Tyndall beam in the anterior chamber is easily | 3a |
| discernible; its intensity is greater than the intensity | |
| of the slit beam as it passes through the lens | |

a, Presence of hypopyon into the aqueous flare leads to a maximum score of 3.

### IRIS

In the following definitions the primary, secondary and tertiary vessels are utilized as an aid to determining a subjective ocular score for iris involvement. The assumption is made that the greater the hyperemia of the vessels and the more the secondary and tertiary vessels are involved, the greater the intensity of iris involvement. The scores range from 0 to +4.

| | Score |
|---|---|
| Normal iris without any hyperemia of the iris vessels. | 0 |
| Occasionally around the 12:00 to 1:00 o'clock position | |
| near the pupillary border and the 6:00 and 7:00 o'clock | |
| position near the pupillary border there is a small area | |
| about 1-3 mm in diameter in which both the secondary and | |
| tertiary vessels are slightly hyperemic | |
| Minimal injection of secondary vessels but not tertiary. | 1 |
| Generally, it is uniform, but may be of greater intensity | |
| at the 1:00 or 6:00 o'clock position. If it is confined | |
| to the 1:00 or 6:00 o'clock position, the tertiary | |
| vessels must be substantially hyperemic | |
| Minimal injection of tertiary vessels and minimal to | 2 |
| moderate injection of the secondary vessels | |
| Moderate injection of the secondary and tertiary vessels | 3 |
| with slight swelling of the iris stroma (this gives the | |
| iris surface a slightly rugose appearance, which is | |
| usually most prominent near the 3:00 and 9:00 o'clock | |
| positions) | |
| Marked injection of the secondary and tertiary vessels | 4b |
| with marked swelling of the iris stroma. The iris appears | |
| rugose; may be accompanied by hemorrhage (hyphema) in | |
| the anterior chamber | |

b Presence of synechia of the iris leads to a maximum score 4.

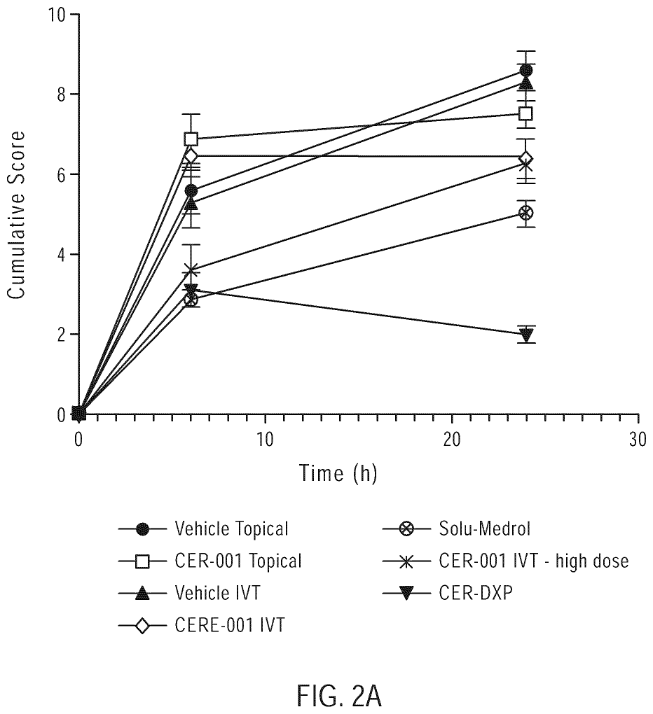

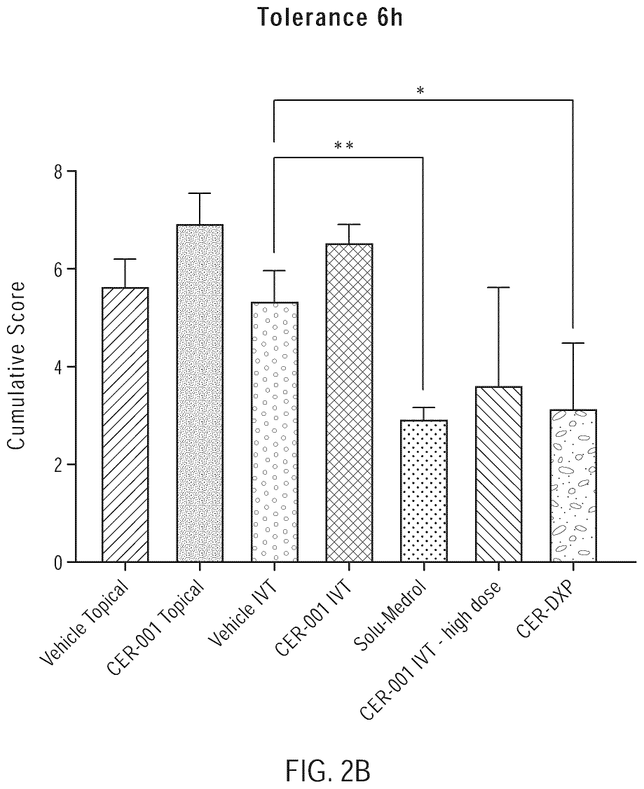

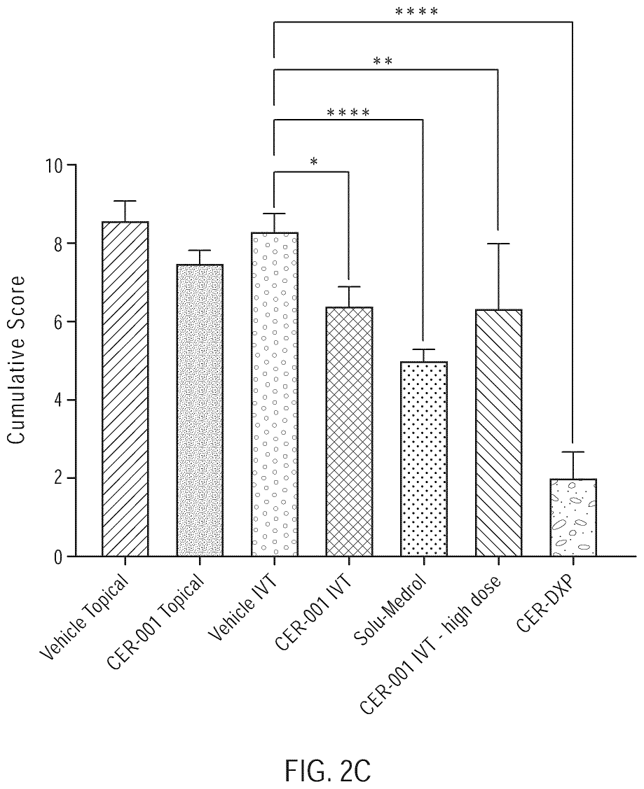

Tolerance for the different treatment groups is shown in FIGS. 2A-2C. Data are expressed as the mean±SEM. One-way ANOVA test results: *: p<0.05; **: p<0.01; ****: p<0.0001.

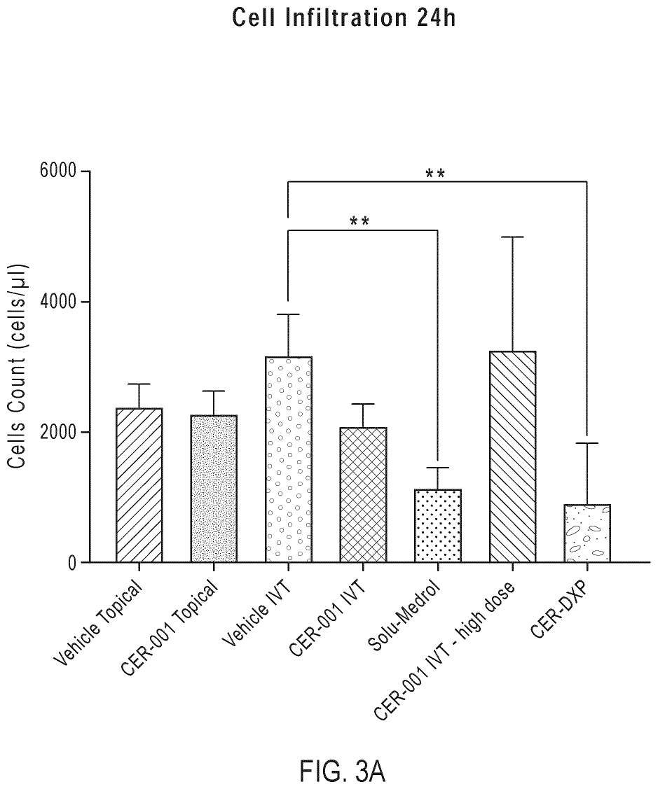

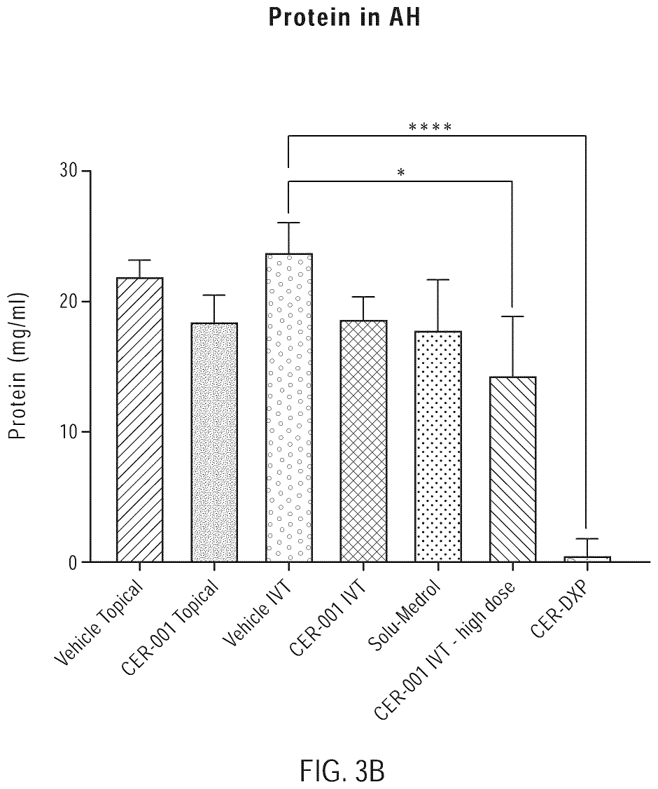

Histological observations were performed to determine the number of infiltrated cells in aqueous humor (AH) after Giemsa staining under microscope. AH cell infiltration is shown in FIG. 3A. Data are expressed as the mean±SEM. One-way ANOVA test results: *: p<0.05; **: p<0.01; ****: p<0.0001.

The protein concentration in AH was measured by a protein-dye binding assay (Bradford test). AH protein content is shown in FIG. 3B. Data are expressed as the mean±SEM. One-way ANOVA test results: *: p<0.05; **: p<0.01; ****: p<0.0001.

For ocular examinations, the total grade consisted of the sum of scores in each of the three categories (conjunctiva (congestion, swelling), aqueous flare, and iris). Group mean and standard deviation values or median values were calculated for AH leukocyte density, AH protein concentration, cumulative ocular clinical score, and body weight.

Transient haze was observed in group 7 (CER-S Cort). Haze scores range from 1 to 3 are defined as follows:
1. The test item is transparent but may create refractive shift when viewing underlying fundus structures.
2. The test item is translucent, permitting only a hazy view of underlying fundus structures.
3. The test item is opaque, obscuring view of underlying fundus structures.

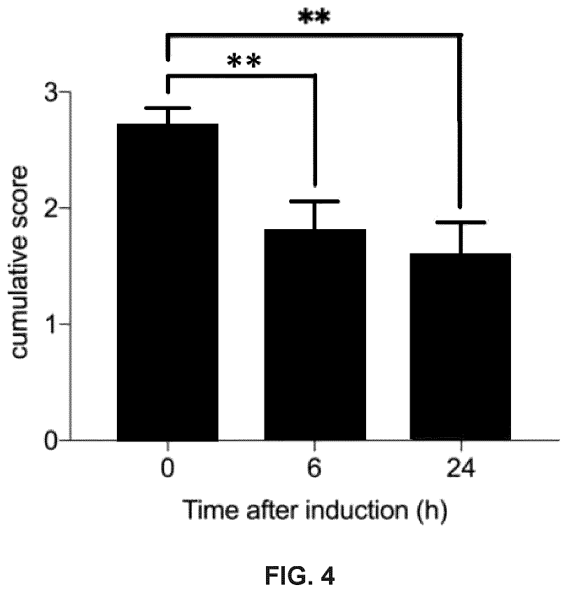

Haze results for group 7 at 0, 6, and 24 hours after induction are shown in FIG. 4. Data are expressed as the mean±SEM. One-way ANOVA test with *: p<0.05; **: p<0.01; ****: p<0.0001.

Tolerance for the different treatment groups is shown in FIGS. 2A-2C, while aqueous humor cell infiltration and protein content are shown in FIGS. 3A-3B, respectively. CER-001 in a single intravitreal administration (including at a high dose of 8 mg/ml) with or without dexamethasone palmitate induced significant tolerance (FIGS. 2A-2C). A positive effect on cell infiltration and protein in the aqueous humor was similarly observed (FIGS. 3A-3B). This Example further supports the use of CER-001 and similar lipid binding protein-based complexes for treating eye diseases such as uveitis and the use of CER-001 and similar lipid binding protein-based complexes to deliver ophthalmic drugs to the eye for treating eye diseases such as uveitis.

### 7.7. Example 7: Efficacy of a Single IVT Administration of CER-001 in a Rabbit Model of DL-α-Aminoadipic Acid-Induced Retinal Neovascularization

The efficacy of CER-001 on retinal DL-α-aminoadipic acid (DL-AAA)-induced neovascularization and permeability was evaluated following a single IVT administration in pigmented rabbits. DL-α-aminoadipic acid (AAA) is a retinal glial cytotoxin that is known to cause glial dysfunction and death, leading to breakdown of the blood-retinal barrier and a persistent retinal neovascularization from 10-12 weeks.

Briefly, pigmented rabbits were randomly assigned to one of the three groups: 8 mg/ml CER-001 (n=6), 15 mg/ml CER-001 (n=4), or vehicle (n=5). 12 weeks before the start of the CER-001 treatment, neovascularization was induced in the right eye of all rabbits by IVT administration of 50 μL of DL-AAA.

Baseline Heidelberg retinal angiography (HRA) assessments were carried out before the administration of CER-001 or vehicle via IVT to the right eye of each rabbit. Following administration of CER-001 or vehicle on day 0, HRA assessments were conducted weekly. Laser flare meter (LFM) and intraocular pressure (IOP) assessments were conducted on day 25 for CER-001 treatment groups and on day 18 for the vehicle treatment group. IOP was used to evaluate treatment-associated changes in intraocular fluid pressure and LFM was used to evaluate intraocular inflammation. Rabbits were euthanized and tissues were sampled on day 40 for CER-001 treatment groups and on day 33 for the vehicle treatment group.

Fluorescein angiography HRA results were used to evaluate vascular leakage. Both CER-001 treatments were associated with a reduction in vascular leakage, corresponding to a decrease in neovascularization and permeability (Table 3). During all HRA assessments, floating bodies were observed in the vitreous of the CER-001 treated rabbits in both groups, whereas they were absent in vehicle-treated rabbits.

TABLE 3
Vascular leakage (% of the baseline)

| | Baseline | Week 1 | Week 2 | Week 3 | Week 4 | Week 5 |
|---|---|---|---|---|---|---|
| | Mean (SD) | Mean (SD) | Mean (SD) | Mean (SD) | Mean (SD) | Mean (SD) |
| CER-001 | 100 (0) | 31 (28) | 22 (43) | 23 (41) | NP | 84 (67) |
| (8 mg/mL) | | | | | | |
| CER-001 | 100 (0) | 25 (16) | 40 (39) | NP | 57 (26) | NP |
| (15 mg/mL) | | | | | | |
| Vehicle | 100 (0) | 99 (41) | 83 (54) | 88 (28) | NP | 128 (33) |

NP = Not performed

Intraocular pressure (IOP) values were comparable between the left (control) and right (treated) eyes of the rabbits in CER-001 (8 mg/mL) and vehicle groups. A slight (<20%) decrease in IOP in the right eye was observed in 50% of the rabbits in the CER-001 (15 mg/mL) group (data not shown). Similarly, laser flare meter (LFM) values for the CER-001 (15 mg/mL)-treated rabbits were elevated relative to the values observed in the lower dose CER-001 group and the vehicle group, as shown in Table 4.

TABLE 4
Laser flare meter values (photons/ms)

| | Right (treated) | Left (untreated) |
|---|---|---|
| | eye Mean (SD) | eye Mean (SD) |
| CER-001 (8 mg/mL) | 44.4 (15.6) | 7.9 (2.7) |
| CER-001 (15 mg/mL) | 108.8 (70.3) | 29.8 (17.9) |
| Vehicle | 23.4 (7.9) | 16.0 (8.0) |

The results of this example indicate that CER-001 can decrease retinal neovascularization and permeability.

### 7.8. Example 8: Efficacy and Ocular Tolerability of CER-001 in a Rabbit Model of Retinal Vascular Hyperpermeability

The efficacy and ocular tolerability of different doses of CER-001 on retinal vascular hyperpermeability was evaluated after a single IVT administration. Briefly, 50 μL of CER-001 (1, 2, 4, or 6 mg/mL), aflibercept (40 mg/mL), or vehicle was administered into the eyes of pigmented rabbits by IVT 2 days prior to the administration of VEGF via IVT. Group 1, n=5, received 6 mg/mL CER-001 in right eyes and 4 mg/mL CER-001 in left eyes. Group 2, n=5, received 2 mg/mL CER-001 in right eyes and 1 mg/mL CER-001 in left eyes. Group 3, n=5, received vehicle in right eyes and aflibercept in left eyes. Vascular leakage was evaluated via fluorophotometry on day 2 after VEGF administration and compared to baseline values determined prior to day-2. Tolerance was assessed by the McDonald-Shadduck scoring system on day-2 (administration of CER-001, aflibercept, or vehicle), day 0 (VEGF administration), and 2 (vascular leakage evaluation). Vascular leakage evaluation results are presented in the Table 5 as ratios of the area under the curve (AUC) for the vitreoretinal segment at day 2 to baseline.

TABLE 5
Vitreoretinal Fluorescence AUC
Ratio: day 2/baseline

| | |
|---|---|
| CER-001 (6 mg/mL) | 24.34 |
| CER-001 (4 mg/mL) | 20.03 |
| CER-001 (2 mg/mL) | 27.87 |
| CER-001 (1 mg/mL) | 24.65 |
| Aflibercept | 1.59 |
| Vehicle | 40.44 |

Fluorophotometry results showed that the administration of the positive control Aflibercept resulted in the lowest vitreoretinal fluorescence ratios. Furthermore, vitreoretinal fluorescence ratio was lower in all CER-001-treated groups relative to the vehicle group. CER-001 administration was associated with floating bodies in the vitreous. McDonald-Shadduck evaluations did not reveal any major clinical signs following the administration of any CER-001 doses. In sum, these results indicate that CER-001 can decrease VEGF-induced retinal vascular permeability.

In a second assessment, the efficacy of CER-001 in a rabbit model of retinal vascular hyperpermeability was evaluated after multiple IVT administrations. This time, 50 μL of CER-001 (8.1 mg/mL) or vehicle was administered into the right eyes of pigmented rabbits by IVT 3 days prior to and one day following the administration of VEGF via IVT. Vascular leakage was evaluated via fluorophotometry on day 2 after VEGF administration.

The treated/untreated eye vitreoretinal fluorescence ratios were lower on day 2 in CER-001 treatment group relative to the vehicle group. Specifically, the mean ratio for the CER-001-treated group was 14±9 and for the vehicle group was 31±18. These results indicate that CER-001 administered via IVT can decrease VEGF-induced retinal vascular permeability.

### 7.9. Example 9: Efficacy of CER-001 Treatment Against Mycobacterium tuberculosis-Induced Chronic Panuveitis in Rabbits

The efficacy of multiple IV administrations of CER-001 against uveitis induced by Mycobacterium tuberculosis was evaluated in albino rabbits. Briefly, each rabbit was injected subcutaneously (SC) with H37Ra to sensitize the rabbits to M. tuberculosis antigen on day 1. Uveitis induction involved IVT injection of H37Ra on days 15 and 30. The rabbits were randomly assigned to a treatment group with either CER-001 (8.1 mg/mL) (n=4) or vehicle (n=4). IV treatments with CER-001 (2 mL/kg, 16.2 mg/kg) or vehicle were conducted on days 1, 5, 9, 13, 17, 21, 25, and 29. Ocular examinations with a slit-lamp were carried out in order to evaluate ocular inflammation in the anterior and the posterior eye sections.

IVT injections of H37Ra antigen resulted in panuveitis in both the anterior and posterior sections of the eyes of vehicle-treated rabbits. For instance, the inflammation in the anterior and posterior eye sections peaked 72 hours after each IVT injection. Rabbits in the CER-001 treatment group displayed less pronounced levels of inflammation in both anterior and posterior eye sections after the first IVT injection. The inflammation score for the posterior eye section did not differ between the two treatment groups after the second IVT injection. The slit-lamp evaluation results are summarized in Table 6 below.

TABLE 6
Ocular observation with a slit-lamp

| | | | | | Prior to 2^{nd} | | |
|---|---|---|---|---|---|---|---|
| | Eye | 24 h after 1^{st} | 72 h after 1^{st} | 7 d after 1^{st} | IVT (same | 24 h after | 72 h after |
| Treatment | Section | IVT | IVT | IVT | day) | 2^{nd} IVT | 2^{nd} IVT |
| CER-001 | Anterior | 1 | 2.5 | 2.75 | 0 | 4 | 2 |
| Vehicle | Anterior | 2 | 4.75 | 2.75 | 0 | 5.25 | 5 |
| CER-001 | Posterior | 0.5 | 0.3 | 0 | 0.3 | 2.7 | 2.3 |
| Vehicle | Posterior | 0.3 | 2.3 | 0.5 | 0.3 | 1.8 | 2.3 |

### 8. SPECIFIC EMBODIMENTS

Various aspects of the present disclosure are described in the embodiments set forth in the following numbered paragraphs of Group 1.

### Group 1:

1. A method of treating a subject with an eye disease, comprising administering to the subject an amount of a lipid binding protein-based complex effective to reduce the severity of the eye disease, optionally complexed with one or more ophthalmic drugs, optionally wherein:
(a) the subject has a cataract or acute anterior uveitis; and/or
(b) the lipid binding protein-based complex is administered by suprachoroidal injection, suprascleral injection, an implant, iontophoresis, or electroporation.

2. The method of embodiment 1, wherein the eye disease is a disease associated with lipid accumulation.

3. The method of embodiment 2, wherein the eye disease is fish-eye disease.

4. The method of embodiment 2, wherein the eye disease is lipid keratopathy.

5. The method of embodiment 4, wherein the lipid keratopathy is secondary lipid keratopathy.

6. The method of embodiment 2, wherein the eye disease is corneal dystrophy, for example an inherited corneal dystrophy, an anterior or superficial corneal dystrophy, a stromal corneal dystrophy, or a posterior corneal dystrophy.

7. The method of any one of embodiments 1 to 6, wherein the subject has corneal opacity and wherein the amount of the lipid binding protein-based complex is an amount effective to reduce the opacity of the subject's cornea(s).

8. The method of embodiment 7, wherein opacity is measured by anterior segment optical coherence tomography (OCT).

9. The method of any one of embodiments 1 to 8, wherein the amount of the lipid binding protein-based complex is an amount effective to improve the subject's contrast sensitivity.

10. The method of any one of embodiments 1 to 9, wherein the amount of the lipid binding protein-based complex is an amount effective to reduce the subject's straylight values.

11. The method of any one of embodiments 1 to 10, wherein the subject is homozygous for an LCAT mutation.

12. The method of any one of embodiments 1 to 10, wherein the subject is heterozygous for an LCAT mutation.

13. The method of any one of embodiments 1 to 2 and 4 to 10, except when depending from embodiment 3, wherein the subject does not have an LCAT deficiency.

14. The method of embodiment 1 or embodiment 2, wherein the subject has a cataract.

15. The method of embodiment 1 or embodiment 2, wherein the eye disease is dry eye.

16. The method of embodiment 15, wherein the dry eye is associated with Meibomian gland dysfunction (MGD).

17. The method of embodiment 16, wherein the MGD is obstructive MGD.

18. The method of embodiment 15, wherein the dry eye is associated with lacrimal gland dysfunction.

19. The method of embodiment 1 or embodiment 2, wherein the eye disease is blepharitis.

20. The method of embodiment 1 or embodiment 2, wherein the eye disease is an inflammatory eye disease.

21. The method of embodiment 1 or embodiment 2, wherein the eye disease is uveitis.

22. The method of embodiment 21, wherein the uveitis is anterior uveitis, intermediate uveitis, posterior uveitis, or panuveitis.

23. The method of embodiment 22, wherein the uveitis is anterior uveitis.

24. The method of claim 23, wherein the uveitis is acute anterior uveitis.

25. The method of embodiment 22, wherein the uveitis is intermediate uveitis.

26 The method of embodiment 22, wherein the uveitis is posterior uveitis.

27. The method of embodiment 22, wherein the uveitis is panuveitis.

28. The method of any one of embodiments 21 to 27, wherein the uveitis is caused by a bacterial infection.

29. The method of embodiment 1 or embodiment 2, wherein the eye disease is macular edema, macular degeneration, retinal detachment, an ocular tumor, a fungal infection, a viral infection, a bacterial infection (e.g., bacterial conjunctivitis or trachoma), multifocal choroiditis, diabetic retinopathy, proliferative vitreoretinopathy (PVR), sympathetic ophthalmia, Vogt Koyanagi-Harada (VKH) syndrome, histoplasmosis, uveal diffusion, vascular occlusion, endophthalmitis, or glaucoma.

30. The method of embodiment 1 or embodiment 2, wherein the eye disease is dry macular degeneration.

31. The method of embodiment 1 or embodiment 2, wherein the eye disease is wet macular degeneration.

32. The method of embodiment 1 or embodiment 2, wherein the eye disease is diabetic retinopathy, optionally wherein the subject has diabetic macular edema.

33. The method of embodiment 1 or embodiment 2, wherein the eye disease is Stargardt disease.

34. The method of any one of embodiments 1 to 33, wherein the subject has impaired vision due to the eye disease and the amount of the lipid binding protein-based complex is an amount which improves the subject's vision.

35. The method of any one of embodiments 1 to 34, wherein the subject has ocular lipid deposits.

36. The method of embodiment 35, wherein the ocular lipid deposits comprise corneal lipid deposits, retinal lipid deposits, palpebral lipid deposits or a combination thereof.

37. The method of embodiment 36, wherein the ocular lipid deposits comprise corneal lipid deposits.

38. The method of embodiment 36 or embodiment 37, wherein the ocular lipid deposits comprise retinal lipid deposits.

39. The method of any one of embodiments 36 to 38, wherein the ocular lipid deposits comprise palpebral lipid deposits.

40. The method of any one of embodiments 35 to 39, wherein the ocular lipid deposits are not calcified.

41. The method of any one of embodiments 35 to 40, wherein the lipid deposits comprise lipid deposits within drusen deposits.

42. The method of any one of embodiments 35 to 40, wherein the lipid deposits comprise lipofuscin granules.

43. The method of any one of embodiments 35 to 40, wherein the lipid deposits comprise cholesterol depots.

44. The method of any one of embodiments 35 to 43, which comprises administering to the subject an amount of a lipid binding protein-based complex effective to reduce the size and/or number of ocular lipid deposits.

45. The method of any one of embodiments 1 to 44, wherein the lipid binding protein-based complex comprises apolipoprotein A-I (ApoA-I), optionally wherein the ApoA-I is not ApoA-I_{Milano}

46. The method of any one of embodiments 1 to 45, wherein the lipid binding protein-based complex does not comprise an apolipoprotein mimetic.

47. The method of any one of embodiments 1 to 46, wherein the lipid binding protein-based complex is a reconstituted HDL or HDL mimetic.

48. The method of embodiment 47, wherein the lipid binding protein-based complex comprises CER-001.

49. The method of embodiment 48, wherein the CER-001 is a lipoprotein complex comprising ApoA-I and phospholipids in a ApoA-I weight:total phospholipid weight ratio of 1:2.7 +/−20% and the phospholipids sphingomyelin and DPPG in a sphingomyelin:DPPG weight:weight ratio of 97:3+/−20%.

50. The method of embodiment 48, wherein the CER-001 is a lipoprotein complex comprising ApoA-I and phospholipids in a ApoA-I weight:total phospholipid weight ratio of 1:2.7 +/−10% and the phospholipids sphingomyelin and DPPG in a sphingomyelin:DPPG weight:weight ratio of 97:3+/−10%.

51. The method of embodiment 48, wherein the CER-001 is a lipoprotein complex comprising ApoA-I and phospholipids in a ApoA-I weight:total phospholipid weight ratio of 1:2.7 and the phospholipids sphingomyelin and DPPG in a sphingomyelin:DPPG weight:weight ratio of 97:3.

52. The method of any one of embodiments 49 to 51, wherein the ApoA-I has the amino acid sequence of amino acids 25-267 of SEQ ID NO:1 of WO 2012/109162.

53. The method of any one of embodiments 49 to 52, wherein the ApoA-I is recombinantly expressed.

54. The method of any one of embodiments 49 to 53, wherein the CER-001 comprises natural sphingomyelin.

55. The method of embodiment 54, wherein the natural sphingomyelin is chicken egg sphingomyelin.

56. The method of any one of embodiments 49 to 53, wherein the CER-001 comprises synthetic sphingomyelin.

57. The method of embodiment 56, wherein the synthetic sphingomyelin is palmitoylsphingomyelin.

58. The method of any one of embodiments 48 to 57, wherein CER-001 is administered in the form of a formulation in which the CER-001 is at least 95% homogeneous.

59. The method of embodiment 58, wherein CER-001 is administered in the form of a formulation in which the CER-001 is at least 97% homogeneous.

60. The method of embodiment 58, wherein CER-001 is administered in the form of a formulation in which the CER-001 is at least 98% homogeneous.

61. The method of embodiment 58, wherein CER-001 is administered in the form of a formulation in which the CER-001 is at least 99% homogeneous.

62. The method of embodiment 47, wherein the lipid binding protein-based complex comprises CSL-111.

63. The method of embodiment 47, wherein the lipid binding protein-based complex comprises CSL-112.

64. The method of embodiment 47, wherein the lipid binding protein-based complex comprises ETC-216.

65. The method of embodiment 47, wherein the lipid binding protein-based complex comprises CER-522.

66. The method of embodiment 47, wherein the lipid binding protein-based complex comprises delipidated HDL.

67. The method of any one of embodiments 1 to 45, wherein the lipid binding protein-based complex is a Cargomer.

68. The method of any one of embodiments 1 to 67, wherein the lipid binding protein-based complex is a carrier for one or more ophthalmic drugs, optionally wherein one or more of the one or more ophthalmic drugs are (i) hydrophobic and/or (ii) poorly water soluble or water insoluble.

69. The method of any one of embodiments 1 to 68, wherein the lipid binding protein-based complex comprises a lipid binding protein-based complex having one or more ophthalmic drugs complexed thereto, optionally wherein one or more of the one or more ophthalmic drugs are (i) hydrophobic and/or (ii) poorly water soluble or water insoluble.

70. The method of embodiment 68 or embodiment 69, wherein the one or more ophthalmic drugs comprise a steroid, a kinase inhibitor, an angiotensin II receptor antagonist, an aldose reductase inhibitor, an immunosuppressant, a carbonic anhydrase inhibitor, an antimicrobial agent, an antiviral agent, an antihistamine, an anti-inflammatory, a prostaglandin analog, or a combination thereof.

71. The method of any one of embodiments 68 to 70, wherein the one or more ophthalmic drugs comprise azithromycin, dexamethasone, difluprednate, estradiol, fluocinolone, fluorometholone, hydrocortisone, loteprednol etabonate, prednisolone, triamcinolone, rimexolone, spironolactone, axitinib, BMS-794833 (N-(4-((2-amino-3-chloropyridin-4-yl)oxy)-3-fluorophenyl)-5-(4-fluorophenyl)-4-oxo-1,4-dihydropyridine-3-carboxamide), carbozantinib, cediranib, dovitinib, lapatinib, lenvatinib, motesanib, nintedanib, orantinib, PD173074 (N-[2-[[4-(Diethylamino)butyl]amino]-6-(3,5-dimethoxyphenyl) pyri-do[2,3-d]pyrimidin-7-yl]-N′-(1,1-dimethylethyl) urea), pazopanib, regorafenib, sorafenib, tofacitinib, ZM323881 (5-((7-Benzyloxyquinazolin-4-yl)amino)-4-fluoro-2-methylphenol), candesartan, irbesartan, losartan, olmesartan, telmisartan, valsartan, 2-methylsorbino, sirolimus, cyclosporine, tacrolimus, acetazolamide, brinzolamide, dorzolamide, ethoxzolamide, diclofenac, indomethacin, nepafenac, latanoprost, travaprost, bimatoprost, or a combination thereof.

72. The method of any one of embodiments 68 to 70, wherein the one or more ophthalmic drugs comprise azithromycin, dexamethasone, difluprednate, estradiol, fluocinolone, fluorometholone, hydrocortisone, loteprednol etabonate, prednisolone, triamcinolone, rimexolone, spironolactone, axitinib, BMS-794833 (N-(4-((2-amino-3-chloropyridin-4-yl)oxy)-3-fluorophenyl)-5-(4-fluorophenyl)-4-oxo-1,4-dihydropyridine-3-carboxamide), carbozantinib, cediranib, dovitinib, lapatinib, lenvatinib, motesanib, nintedanib, orantinib, PD173074 (N-[2-[[4-(Diethylamino)butyl]amino]-6-(3,5-dimethoxyphenyl) pyri-do[2,3-d]pyrimidin-7-yl]-N′-(1,1-dimethylethyl) urea), pazopanib, regorafenib, sorafenib, tofacitinib, ZM323881 (5-((7-Benzyloxyquinazolin-4-yl)amino)-4-fluoro-2-methylphenol), candesartan, irbesartan, losartan, olmesartan, telmisartan, valsartan, 2-methylsorbino, sirolimus, cyclosporine, tacrolimus, acetazolamide, brinzolamide, dorzolamide, ethoxzolamide, methazolamide, acyclovir, chloramphenicol, chlortetracycline, ciprofloxacin, fusidic acid, gancyclovir, norfloxacin, ofloxacin, tetracycline, zidovudine, levocabastine, bromfenac, diclofenac, indomethacin, nepafenac, latanoprost, travaprost, bimatoprost, dexamethasone palmitate, or a combination thereof.

73. The method of any one of embodiments 68 to 72, wherein the one or more ophthalmic drugs comprise azithromycin.

74. The method of any one of embodiments 68 to 73, wherein the one or more ophthalmic drugs comprise spironolactone.

75. The method of any one of embodiments 68 to 74, wherein the one or more ophthalmic drugs comprise dexamethasone palmitate.

76. The method of any one of embodiments 68 to 75, wherein the one or more ophthalmic drugs comprise cyclosporine.

77. The method of any one of embodiments 68 to 76, wherein the one or more ophthalmic drugs comprise latanoprost, travaprost, bimatoprost, tafluprost, or a combination thereof.

78. The method of embodiment 77, wherein the one or more ophthalmic drugs comprise latanoprost.

79. The method of any one of embodiments 68 to 78, wherein the one or more ophthalmic drugs comprise dexamethasone.

80. The method of any one of embodiments 68 to 79, wherein the one or more ophthalmic drugs comprise loteprednol etabonate

81. The method of any one of embodiments 68 to 80, wherein the one or more ophthalmic drugs comprise triamcinolone.

82. The method of any one of embodiments 68 to 81, wherein the one or more ophthalmic drugs comprise acyclovir.

83. The method of any one of embodiments 68 to 82, wherein the one or more ophthalmic drugs comprise travaprost.

84. The method of any one of embodiments 68 to 83, wherein the one or more ophthalmic drugs comprise bimatoprost.

85. The method of any one of embodiments 68 to 84, wherein the one or more ophthalmic drugs comprise tafluprost.

86. The method of any one of embodiments 68 to 85, wherein the one or more ophthalmic drugs comprise pazopanib.

87. The method of any one of embodiments 68 to 86, wherein the one or more ophthalmic drugs comprise sirolimus.

88. The method of any one of embodiments 68 to 86, wherein the one or more ophthalmic drugs comprise tacrolimus.

89. The method of any one of embodiments 68 to 86, wherein the one or more ophthalmic drugs comprise nepafenac.

90. The method of any one of embodiments 1 to 46, wherein the lipid binding protein-based complex is an Apomer.

91. The method of any one of embodiments 1 to 90, wherein the lipid binding protein-based complex is administered peripherally, optionally by infusion.

92. The method of embodiment 91, wherein the lipid binding protein-based complex is administered according to a dosing regimen which comprises:
(a) an induction regimen; and/or
(b) a consolidation regimen; and/or
(c) a maintenance regimen,
optionally wherein the lipid binding protein-based complex comprises CER-001.

93. The method of embodiment 92, which comprises administering one or more doses of the lipid binding protein-based complex according to an induction regimen.

94. The method of embodiment 93, wherein the induction regimen comprises administering multiple doses of the lipid binding protein-based complex to the subject.

95. The method of embodiment 94, wherein the induction regimen comprises administering at least three doses of the lipid binding protein-based complex to the subject.

96. The method of embodiment 94 or embodiment 95, in which multiple doses in the induction regimen are separated by 1 or more days.

97. The method of any one any one of embodiments 93 to 96, wherein the doses following the initial dose of the induction regimen are separated by no more than 3 days.

98. The method of embodiment 97, wherein the doses following the initial dose of the induction regimen are separated by one to three days.

99. The method of embodiment 97, wherein the doses following the initial dose of the induction regimen are separated by two to three days.

100. The method of embodiment 97, wherein the doses following the initial dose of the induction regimen are separated by one to two days.

101. The method of any one of embodiments 93 to 100, wherein the induction regimen is for a duration of at least one week.

102. The method of embodiment 101, wherein the induction regimen is for a duration of two weeks.

103. The method of embodiment 101, wherein the induction regimen is for a duration of three weeks.

104. The method of any one of embodiments 93 to 103, in which the induction regimen comprises administering to the subject three doses of the lipid binding protein-based complex per week.

105. The method of any one of embodiments 93 to 103, wherein the induction regimen comprises administering four or more doses of the lipid binding protein-based complex to the subject.

106. The method of any one of embodiments 93 to 103, wherein the induction regimen comprises administering five or more doses of the lipid binding protein-based complex to the subject.

107. The method of any one of embodiments 93 to 103, wherein the induction regimen comprises administering six or more doses of the lipid binding protein-based complex to the subject.

108. The method of any one of embodiments 93 to 103, wherein the induction regimen comprises administering seven or more doses of the lipid binding protein-based complex to the subject.

109. The method of any one of embodiments 93 to 103, wherein the induction regimen comprises administering eight or more doses of the lipid binding protein-based complex to the subject.

110. The method of any one of embodiments 93 to 103, wherein the induction regimen comprises administering nine or more doses of the lipid binding protein-based complex to the subject.

111. The method of embodiment 110, wherein the induction regimen comprises administering the first dose of the lipid binding protein-based complex to the subject on day 1 and administering subsequent doses of the induction regimen to the subject on days 2, 4, 7, 9, 11, 14, 16, and 18.

112. The method of any one of embodiments 93 to 103, wherein the induction regimen comprises administering ten or more doses of the lipid binding protein-based complex to the subject.

113. The method of embodiment 92, which does not include an induction regimen.

114. The method of any one of embodiments 92 to 113, which comprises administering to the subject one or more doses of the lipid binding protein-based complex according to a consolidation regimen.

115. The method of embodiment 114, wherein the consolidation regimen comprises administering multiple doses of the lipid binding protein-based complex to the subject.

116. The method of embodiment 115, in which multiple doses in the consolidation regimen are separated by 2 or more days.

117. The method of any one of embodiments 114 to 116, wherein the consolidation regimen comprises administering at least two doses of the lipid binding protein-based complex to the subject in one week.

118. The method of any one of embodiments 114 to 117, wherein the doses of the consolidation regimen are separated by no more than four days.

119. The method of any one of embodiments 114 to 118, wherein the doses of the consolidation regimen are separated from one another by three or four days.

120. The method of any one of embodiments 114 to 119, wherein the consolidation regimen is for a duration of at least 3 weeks.

121. The method of any one of embodiments 114 to 120, wherein the consolidation regimen comprises administering three or more doses of the lipid binding protein-based complex to the subject.

122. The method of any one of embodiments 114 to 120, wherein the consolidation regimen comprises administering four or more doses of the lipid binding protein-based complex to the subject.

123. The method of any one of embodiments 114 to 120, wherein the consolidation regimen comprises administering five or more doses of the lipid binding protein-based complex to the subject.

124. The method of any one of embodiments 114 to 120, wherein the consolidation regimen comprises administering six or more doses of the lipid binding protein-based complex to the subject.

125. The method of embodiment 124, wherein the consolidation regimen comprises administering six doses of the lipid binding protein-based complex to the subject.

126. The method of embodiment 125, wherein the consolidation regimen comprises administering the six doses of the lipid binding protein-based complex to the subject on days 21, 24, 28, 31, 35, and 38 following an induction regimen which begins on day 1.

127. The method of any one of embodiments 114 to 120, wherein the consolidation regimen comprises administering seven or more doses of the lipid binding protein-based complex to the subject.

128. The method of any one of embodiments 114 to 120, wherein the consolidation regimen comprises administering eight or more doses of the lipid binding protein-based complex to the subject.

129. The method of any one of embodiments 114 to 120, wherein the consolidation regimen comprises administering nine or more doses of the lipid binding protein-based complex to the subject.

130. The method of any one of embodiments 114 to 120, wherein the consolidation regimen comprises administering ten or more doses of the lipid binding protein-based complex to the subject.

131. The method of any one of embodiments 92 to 113, which does not include a consolidation regimen.

132. The method of any one of embodiments 92 to 131, which comprises administering to the subject multiple doses of the lipid binding protein-based complex according to a maintenance regimen.

133. The method of embodiment 132, wherein the maintenance regimen comprises administering a dose of the lipid binding protein-based complex to the subject once every 3 or more days.

134. The method of embodiment 132, wherein the maintenance regimen comprises administering a dose of the lipid binding protein-based complex to the subject once every 5 or more days.

135. The method of embodiment 132, wherein the maintenance regimen comprises administering a dose of the lipid binding protein-based complex to the subject weekly.

136. The method of embodiment 135, wherein the doses of the maintenance regimen are administered +/−2 days around the strict weekly date.

137. The method of embodiment 132, wherein the maintenance regimen comprises administering a dose of the lipid binding protein-based complex to the subject twice weekly.

138. The method of any one of embodiments 132 to 137, wherein the maintenance regimen comprises administering the lipid binding protein-based complex to the subject for at least one month.

139. The method of any one of embodiments 132 to 137, wherein the maintenance regimen comprises administering the lipid binding protein-based complex to the subject for at least two months.

140. The method of any one of embodiments 132 to 137, wherein the maintenance regimen comprises administering the lipid binding protein-based complex to the subject for at least three months.

141. The method of any one of embodiments 132 to 137, wherein the maintenance regimen comprises administering the lipid binding protein-based complex to the subject for at least six months.

142. The method of any one of embodiments 132 to 137, wherein the maintenance regimen comprises administering the lipid binding protein-based complex to the subject for at least nine months.

143. The method of any one of embodiments 132 to 137, wherein the maintenance regimen comprises administering the lipid binding protein-based complex to the subject for at least a year.

144. The method of any one of embodiments 132 to 137, wherein the maintenance regimen comprises administering the lipid binding protein-based complex to the subject for at least 18 months.

145. The method of any one of embodiments 132 to 137, wherein the maintenance regimen comprises administering the lipid binding protein-based complex to the subject for at least 2 years.

146. The method of any one of embodiments 132 to 137, wherein the maintenance regimen comprises administering the lipid binding protein-based complex to the subject indefinitely.

147. The method of any one of embodiments 132 to 137, wherein the maintenance regimen comprises administering the lipid binding protein-based complex to the subject for 16 or more weeks.

148. The method of any one of embodiments 132 to 137, wherein the maintenance regimen comprises administering the lipid binding protein-based complex to the subject for 20 or more weeks.

149. The method of any one of embodiments 132 to 137, wherein the maintenance regimen comprises administering the lipid binding protein-based complex to the subject for 30 or more weeks.

150. The method of any one of embodiments 132 to 137, wherein the maintenance regimen comprises administering the lipid binding protein-based complex to the subject for 40 or more weeks.

151. The method of any one of embodiments 92 to 150, wherein the dose of the lipid binding protein-based complex administered in the induction regimen is 4 to 30 mg/kg (on a protein weight basis).

152. The method of any one of embodiments 92 to 150, wherein the dose of the lipid binding protein-based complex administered in the induction regimen is 5 to 15 mg/kg (on a protein weight basis).

153. The method of any one of embodiments 92 to 150, wherein the dose of the lipid binding protein-based complex administered in the induction regimen is 10 to 20 mg/kg (on a protein weight basis).

154. The method of any one of embodiments 92 to 150, wherein the dose of the lipid binding protein-based complex administered in the induction regimen is 15 to 25 mg/kg (on a protein weight basis).

155. The method of any one of embodiments 92 to 150, wherein the dose of the lipid binding protein-based complex administered in the induction regimen is 8 mg/kg (on a protein weight basis).

156. The method of any one of embodiments 92 to 150, wherein the dose of the lipid binding protein-based complex administered in the induction regimen is 10 mg/kg (on a protein weight basis).

157. The method of any one of embodiments 92 to 150, wherein the dose of the lipid binding protein-based complex administered in the induction regimen is 300 mg to 3000 mg.

158. The method of any one of embodiments 92 to 150, wherein the dose of the lipid binding protein-based complex administered in the induction regimen is 300 mg to 1500 mg.

159. The method of any one of embodiments 92 to 150, wherein the dose of the lipid binding protein-based complex administered in the induction regimen is 400 mg to 1500 mg.

160. The method of any one of embodiments 92 to 150, wherein the dose of the lipid binding protein-based complex administered in the induction regimen is 500 mg to 1200 mg. 161. The method of any one of embodiments 92 to 150, wherein the dose of the lipid binding protein-based complex administered in the induction regimen is 500 mg to 1000 mg.

162. The method of any one of embodiments 92 to 161, wherein the dose of the lipid binding protein-based complex administered in the consolidation regimen is 4 to 30 mg/kg (on a protein weight basis).

163. The method of any one of embodiments 92 to 161, wherein the dose of the lipid binding protein-based complex administered in the consolidation regimen is 5 to 15 mg/kg (on a protein weight basis).

164. The method of any one of embodiments 92 to 161, wherein the dose of the lipid binding protein-based complex administered in the consolidation regimen is 10 to 20 mg/kg (on a protein weight basis).

165. The method of any one of embodiments 92 to 161, wherein the dose of the lipid binding protein-based complex administered in the consolidation regimen is 15 to 25 mg/kg (on a protein weight basis).

166. The method of any one of embodiments 92 to 161, wherein the dose of the lipid binding protein-based complex administered in the consolidation regimen is 8 mg/kg (on a protein weight basis).

167. The method of any one of embodiments 92 to 161, wherein the dose of the lipid binding protein-based complex administered in the consolidation regimen is 10 mg/kg (on a protein weight basis).

168. The method of any one of embodiments 92 to 161, wherein the dose of the lipid binding protein-based complex administered in the consolidation regimen is 300 mg to 3000 mg.

169. The method of any one of embodiments 92 to 161, wherein the dose of the lipid binding protein-based complex administered in the consolidation regimen is 300 mg to 1500 mg.

170. The method of any one of embodiments 92 to 161, wherein the dose of the lipid binding protein-based complex administered in the consolidation regimen is 400 mg to 1500 mg.

171. The method of any one of embodiments 92 to 161, wherein the dose of the lipid binding protein-based complex administered in the consolidation regimen is 500 mg to 1200 mg.

172. The method of any one of embodiments 92 to 161, wherein the dose of the lipid binding protein-based complex administered in the consolidation regimen is 500 mg to 1000 mg.

173. The method of any one of embodiments 92 to 172, wherein the dose of the lipid binding protein-based complex administered in the maintenance regimen is 4 to 30 mg/kg (on a protein weight basis).

174. The method of any one of embodiments 92 to 172, wherein the dose of the lipid binding protein-based complex administered in the maintenance regimen is 5 to 15 mg/kg (on a protein weight basis).

175. The method of any one of embodiments 92 to 172, wherein the dose of the lipid binding protein-based complex administered in the maintenance regimen is 10 to 20 mg/kg (on a protein weight basis).

176. The method of any one of embodiments 92 to 172, wherein the dose of the lipid binding protein-based complex administered in the maintenance regimen is 15 to 25 mg/kg (on a protein weight basis).

177. The method of any one of embodiments 92 to 172, wherein the dose of the lipid binding protein-based complex administered in the maintenance regimen is 8 mg/kg (on a protein weight basis).

178. The method of any one of embodiments 92 to 172, wherein the dose of the lipid binding protein-based complex administered in the maintenance regimen is 10 mg/kg (on a protein weight basis).

179. The method of any one of embodiments 92 to 172, wherein the dose of the lipid binding protein-based complex administered in the maintenance regimen is 20 mg/kg (on a protein weight basis).

180. The method of any one of embodiments 92 to 172, wherein the dose of the lipid binding protein-based complex administered in the maintenance regimen is 300 mg to 3000 mg.

181. The method of any one of embodiments 92 to 172, wherein the dose of the lipid binding protein-based complex administered in the maintenance regimen is 300 mg to 1500 mg.

182. The method of any one of embodiments 92 to 172, wherein the dose of the lipid binding protein-based complex administered in the maintenance regimen is 400 mg to 1500 mg.

183. The method of any one of embodiments 92 to 172, wherein the dose of the lipid binding protein-based complex administered in the maintenance regimen is 500 mg to 1200 mg.

184. The method of any one of embodiments 92 to 172, wherein the dose of the lipid binding protein-based complex administered in the maintenance regimen is 500 mg to 1000 mg.

185. The method of any one of embodiments 92 to 184, which comprises both an induction regimen and a maintenance regimen.

186. The method of embodiment 185, wherein the dose of the lipid binding protein-based complex administered in the induction regimen and the dose of the lipid binding protein-based complex administered in the maintenance regimen are the same.

187. The method of embodiment 185, wherein the dose of the lipid binding protein-based complex administered in the induction regimen and the dose of the lipid binding protein-based complex administered in the maintenance regimen are different.

188. The method of embodiment 187, wherein the dose of the lipid binding protein-based complex administered in the maintenance regimen is greater than the dose of the lipid binding protein-based complex administered in the induction regimen.

189. The method of embodiment 188, wherein the dose of the lipid binding protein-based complex administered in the maintenance regimen is 1.5 to 3 times the dose of the lipid binding protein-based complex administered in the induction regimen.

190. The method of embodiment 188, wherein the dose of the lipid binding protein-based complex administered in the maintenance regimen is 2 times the dose administered in the induction regimen.

191. The method of any one of embodiments 1 to 90, wherein the lipid binding protein-based complex is administered locally.

192. The method of embodiment 191, wherein the lipid binding protein-based complex is administered intraocularly.

193. The method of embodiment 192, wherein the lipid binding protein-based complex is administered by intraocular injection.

194. The method of embodiment 193, wherein the intraocular injection is suprachoroidal injection.

195. The method of embodiment 193, wherein the intraocular injection is suprascleral injection.

196. The method of embodiment 193, wherein the intraocular injection is intra-vitreal injection.

197. The method of embodiment 193, wherein the intraocular injection is sub-conjunctival injection.

198. The method of embodiment 193, wherein the intraocular injection is parabulbar injection.

199. The method of embodiment 193, wherein the intraocular injection is peribulbar injection.

200. The method of embodiment 193, wherein the intraocular injection is retro-bulbar injection.

201. The method of embodiment 191 or embodiment 192, wherein the lipid binding protein-based complex is administered as an implant.

202. The method of embodiment 201, wherein the implant is a disc.

203. The method of embodiment 201, wherein the implant is a sheet.

204. The method of embodiment 201, wherein the implant is a plug.

205. The method of embodiment 201, wherein the implant is a rod.

206. The method of embodiment 201, wherein the implant is a pellet.

207. The method of any one of embodiments 201 to 206, wherein the implant is biodegradable.

208. The method of any one of embodiments 201 to 206, wherein the implant is not biodegradable.

209. The method of embodiment 191, wherein the lipid binding protein-based complex is administered by iontophoresis.

210. The method of embodiment 209, wherein the lipid binding protein-based complex is administered by transcorneal iontophoresis.

211. The method of embodiment 209, wherein the lipid binding protein-based complex is administered by transscleral iontophoresis.

212. The method of embodiment 191, wherein the lipid binding protein-based complex is administered by electroporation.

213. The method of embodiment 191, wherein the lipid binding protein-based complex is administered topically.

214. The method of embodiment 213, wherein the lipid binding protein-based complex is formulated as an eye drop.

215. The method of any one of embodiments 191 to 214, wherein the lipid binding protein-based complex is administered according to a dosing regimen which comprises:
(a) an induction regimen; and/or
(b) a consolidation regimen; and/or
(c) a maintenance regimen,
optionally wherein the lipid binding protein-based complex is CER-001.

216. The method of embodiment 215, which comprises an induction regimen.

217. The method of embodiment 215 or embodiment 216, which comprises a consolidation regimen.

218. The method of any one of embodiments 215 to 217, which comprises a maintenance regimen.

219. The method of any one of embodiments 1 to 218, wherein an antihistamine is administered prior to administration of one or more of the lipid binding protein-based complex doses.

220. The method of any one of embodiments 1 to 219, wherein the subject is also treated with a lipid control medication.

221. The method of embodiment 220, wherein the lipid control medication comprises a statin.

222. The method of embodiment 221, wherein the statin is atorvastatin, rosuvastatin, simvastatin, fluvastatin, lovastatin, or pravastatin.

223. The method of any one of embodiments 220 to 222, wherein the lipid control medication comprises a cholesterol absorption inhibitor.

224. The method of embodiment 223, wherein the cholesterol absorption inhibitor is ezetimibe.

225. The method of any one of embodiments 220 to 224, wherein the lipid control medication comprises niacin.

226. The method of any one of embodiments 220 to 225, wherein the lipid control medication comprises aspirin.

227. The method of any one of embodiments 220 to 226, wherein the lipid control medication comprises a proprotein convertase subtilisin/kexin type 9 (PCSK9) inhibitor.

228. The method of embodiment 227, wherein the PCSK9 inhibitor is an antibody.

229. The method of embodiment 228, wherein the antibody is alirocumab, bococizumab, evolocumab, 1D05-IgG2 or LY3015014.

230. The method of embodiment 227, wherein the PCSK9 inhibitor is RNAi therapeutic.

231. The method of embodiment 230, wherein the RNAi therapeutic is ALN-PCSSC.

232. The method of any one of embodiments 220 to 231, further comprising administering a therapeutically effective amount of the lipid control medication to the subject.

233. The method of any one of embodiments 1 to 232, wherein the subject is also treated with a standard of care therapy for the eye disease.

234. The method of embodiment 233, further comprising administering the standard of care therapy to the subject.

235. The method of any one of embodiments 1 to 234, wherein the subject is also treated with an antihypertensive medication, optionally wherein the antihypertensive medication comprises one, two, or all three of amlodipine, urapidil, and furosemide.

236. The method of any one of embodiments 1 to 235, wherein the lipid binding protein-based complex does not comprise and is not administered with a cell-penetrating peptide.

237. The method of any one of embodiments 1 to 236, wherein the lipid binding protein-based complex does not comprise and is not administered with a chemical penetration enhancer.

238. The method of any one of embodiments 1 to 237, wherein the lipid binding protein-based complex does not comprise and is not administered with a cytophilic peptide.

239. A composition comprising a lipid binding protein-based complex and one or more ophthalmic drugs, wherein the composition is produced by a process comprising thermal cycling a mixture comprising the lipid binding protein-based complex and the one or more ophthalmic drugs, optionally wherein one or more of the one or more ophthalmic drugs are (i) hydrophobic and/or (ii) poorly water soluble or water insoluble.

240. The composition of embodiment 239, wherein the thermal cycling comprises
(a) heating the mixture from a temperature in a first temperature range to a temperature in a second temperature range,
(b) cooling the mixture of (a) from a temperature in the second temperature range to a temperature in the first temperature range; and
(c) optionally repeating steps (a) and (b) at least once.

241. The composition of embodiment 240, wherein the thermal cycling comprising repeating steps (a) and (b) one time.

242. The composition of embodiment 240, wherein the thermal cycling comprising repeating steps (a) and (b) two times.

243. The composition of embodiment 240, wherein the thermal cycling comprising repeating steps (a) and (b) three times.

244. The composition of embodiment 240, wherein the thermal cycling comprising repeating steps (a) and (b) four times.

245. The composition of embodiment 240, wherein the thermal cycling comprising repeating steps (a) and (b) five times.

246. The composition of any one of embodiments 240 to 245, wherein the first temperature range is 30° C. to 45° C.

247. The composition of embodiment 246, wherein the temperature in the first temperature range is 37° C.

248. The composition of any one of embodiments 240 to 247, wherein the second temperature range is 50° C. to 65° C.

249. The composition of embodiment 248, wherein the temperature in the second temperature range is 55° C.

250. The composition of any one of embodiments 239 to 249, wherein the thermal cycling comprises thermal cycling the mixture between 37° C. and 55° C.

251. A composition comprising a lipid binding protein-based complex and one or more ophthalmic drugs complexed thereto, optionally wherein one or more of the one or more ophthalmic drugs are (i) hydrophobic and/or (ii) poorly water soluble or water insoluble.

252. The composition of any one of embodiments 239 to 251, wherein the lipid binding protein-based complex comprises a lipid binding protein molecule described in Section 6.1.4.

253. The composition of any one of embodiments 239 to 251, wherein the lipid binding protein-based complex comprises apolipoprotein A-I (ApoA-I), optionally wherein the ApoA-I is not ApoA-I_{Milano}.

254. The composition of any one of embodiments 239 to 253, wherein the lipid binding protein-based complex does not comprise an apolipoprotein mimetic.

255. The composition of any one of embodiments 239 to 254, wherein the lipid binding protein-based complex comprises one or more amphipathic molecules described in Section 6.1.5.

256. The composition of any one of embodiments 239 to 255, wherein the lipid binding protein-based complex comprises one or more neutral lipids.

257. The composition of embodiment 256, wherein the one or more neutral lipids comprises a sphingomyelin.

258. The composition of any one of embodiments 239 to 257, wherein the lipid binding protein-based complex comprises one or more negatively charged lipids.

259. The composition of embodiment 258, wherein the one or more negatively charged lipids comprise 1,2-dipalmitoyl-sn-glycero-3-[phospho-rac-(1-glycerol) (DPPG) or a salt thereof.

260. The composition of any one of embodiments 239 to 259, wherein the lipid binding protein-based complex is a reconstituted HDL or HDL mimetic.

261. The composition of embodiment 260, wherein the lipid binding protein-based complex is CER-001.

262. The composition of embodiment 260, wherein the lipid binding protein-based complex is CSL-111.

263. The composition of embodiment 260, wherein the lipid binding protein-based complex is CSL-112.

264. The composition of embodiment 260, wherein the lipid binding protein-based complex is ETC-216.

265. The composition of embodiment 260, wherein the lipid binding protein-based complex is CER-522.

266. The composition of embodiment 260, wherein the lipid binding protein-based complex is delipidated HDL.

267. The composition of any one of embodiments 239 to 259, wherein the lipid binding protein-based complex is an Apomer.

268. The composition of any one of embodiments 239 to 259, wherein the lipid binding protein-based complex is a Cargomer.

269. The composition of any one of embodiments 239 to 268, wherein the one or more ophthalmic drugs comprise a steroid, a kinase inhibitor, an angiotensin II receptor antagonist, an aldose reductase inhibitor, an immunosuppressant, a carbonic anhydrase inhibitor, an antimicrobial agent, an antiviral agent, an antihistamine, an anti-inflammatory, or a combination thereof.

270. The composition of any one of embodiments 239 to 269, wherein the one or more ophthalmic drugs comprise azithromycin, dexamethasone, difluprednate, estradiol, fluocinolone, fluorometholone, hydrocortisone, loteprednol etabonate, prednisolone, triamcinolone, rimexolone, spironolactone, axitinib, BMS-794833 (N-(4-((2-amino-3-chloropyridin-4-yl)oxy)-3-fluorophenyl)-5-(4-fluorophenyl)-4-oxo-1,4-dihydropyridine-3-carboxamide), carbozantinib, cediranib, dovitinib, lapatinib, lenvatinib, motesanib, nintedanib, orantinib, PD173074 (N-[2-[[4-(Diethylamino)butyl]amino]-6-(3,5-dimethoxyphenyl) pyri-do[2,3-d]pyrimidin-7-yl]-N′-(1,1-dimethylethyl) urea), pazopanib, regorafenib, sorafenib, tofacitinib, ZM323881 (5-((7-Benzyloxyquinazolin-4-yl)amino)-4-fluoro-2-methylphenol), candesartan, irbesartan, losartan, olmesartan, telmisartan, valsartan, 2-methylsorbino, sirolimus, cyclosporine, tacrolimus, acetazolamide, brinzolamide, dorzolamide, ethoxzolamide, methazolamide, acyclovir, chloramphenicol, chlortetracycline, ciprofloxacin, fusidic acid, gancyclovir, norfloxacin, ofloxacin, tetracycline, zidovudine, levocabastine, bromfenac, diclofenac, indomethacin, nepafenac, or a combination thereof.

271. The composition of any one of embodiments 239 to 269, wherein the one or more ophthalmic drugs comprise azithromycin, dexamethasone, difluprednate, estradiol, fluocinolone, fluorometholone, hydrocortisone, loteprednol etabonate, prednisolone, triamcinolone, rimexolone, spironolactone, axitinib, BMS-794833 (N-(4-((2-amino-3-chloropyridin-4-yl)oxy)-3-fluorophenyl)-5-(4-fluorophenyl)-4-oxo-1,4-dihydropyridine-3-carboxamide), carbozantinib, cediranib, dovitinib, lapatinib, lenvatinib, motesanib, nintedanib, orantinib, PD173074 (N-[2-[[4-(Diethylamino)butyl]amino]-6-(3,5-dimethoxyphenyl) pyri-do[2,3-d]pyrimidin-7-yl]-N′-(1,1-dimethylethyl) urea), pazopanib, regorafenib, sorafenib, tofacitinib, ZM323881 (5-((7-Benzyloxyquinazolin-4-yl)amino)-4-fluoro-2-methylphenol), candesartan, irbesartan, losartan, olmesartan, telmisartan, valsartan, 2-methylsorbino, sirolimus, cyclosporine, tacrolimus, acetazolamide, brinzolamide, dorzolamide, ethoxzolamide, methazolamide, acyclovir, chloramphenicol, chlortetracycline, ciprofloxacin, fusidic acid, gancyclovir, norfloxacin, ofloxacin, tetracycline, zidovudine, levocabastine, bromfenac, diclofenac, indomethacin, nepafenac, dexamethasone palmitate, or a combination thereof.

272. The composition of embodiment 270 or embodiment 271, wherein the one or more ophthalmic drugs comprise azithromycin, optionally wherein the concentration of azithromycin in the composition is 10 mg/ml.

273. The composition of any one of embodiments 270 to 272, wherein the one or more ophthalmic drugs comprise spironolactone, optionally wherein the concentration of spironolactone in the composition is 1.5 mg/ml.

274. The composition of any one of embodiments 270 to 273, wherein the one or more ophthalmic drugs comprise dexamethasone palmitate, optionally wherein the concentration of dexamethasone palmitate in the composition is 1 mg/ml.

275. The composition of any one of embodiments 270 to 274, wherein the one or more ophthalmic drugs comprise cyclosporine, optionally wherein the concentration of cyclosporine in the composition is 1 mg/ml.

276. The composition of any one of embodiments 239 to 275, wherein the one or more ophthalmic drugs comprise latanoprost, travaprost, bimatoprost, tafluprost, or a combination thereof.

277. The composition of embodiment 276, wherein the one or more ophthalmic drugs comprise latanoprost.

278. The composition of any one of embodiments 239 to 277, wherein the one or more ophthalmic drugs comprise dexamethasone.

279. The composition of any one of embodiments 239 to 278, wherein the one or more ophthalmic drugs comprise loteprednol etabonate

280. The composition of any one of embodiments 239 to 279, wherein the one or more ophthalmic drugs comprise triamcinolone.

281. The composition of any one of embodiments 239 to 280, wherein the one or more ophthalmic drugs comprise acyclovir.

282. The composition of any one of embodiments 239 to 281, wherein the one or more ophthalmic drugs comprise travaprost.

283. The composition of any one of embodiments 239 to 282, wherein the one or more ophthalmic drugs comprise bimatoprost.

284. The composition of any one of embodiments 239 to 283, wherein the one or more ophthalmic drugs comprise tafluprost.

285. The composition of any one of embodiments 239 to 284, wherein the one or more ophthalmic drugs comprise pazopanib.

286. The composition of any one of embodiments 239 to 285, wherein the one or more ophthalmic drugs comprise sirolimus.

287. The composition of any one of embodiments 239 to 286, wherein the one or more ophthalmic drugs comprise tacrolimus.

288. The composition of any one of embodiments 239 to 287, wherein the one or more ophthalmic drugs comprise nepafenac.

289. The composition of any one of embodiments 239 to 288, which does not comprise a cell-penetrating peptide.

290. The composition of any one of embodiments 239 to 289, which does not comprise a chemical penetration enhancer.

291. The composition of any one of embodiments 239 to 290, which does not comprise a cytophilic peptide.

292. The composition of any one of embodiments 239 to 291, which is a pharmaceutical composition further comprising one or more buffers, preservatives, excipients, diluents, or a combination thereof.

293. The composition of any one of embodiments 239 to 292 for use in the method of any one of embodiments 1 to 238.

294. A process for producing a composition comprising a lipid binding protein-based complex and one or more ophthalmic drugs, optionally wherein the composition is the composition of any one of embodiments 251 to 293, the process comprising thermal cycling a mixture comprising the lipid binding protein-based complex and the one or more ophthalmic drugs.

295. The process of embodiment 294, wherein the thermal cycling comprises
(a) heating the mixture from a temperature in a first temperature range to a temperature in a second temperature range,
(b) cooling the mixture of (a) from a temperature in the second temperature range to a temperature in the first temperature range; and
(c) optionally repeating steps (a) and (b) at least once.

296. The process of embodiment 295, wherein steps (a) and (b) are repeated one time.

297. The process of embodiment 295, wherein steps (a) and (b) are repeated two times.

298. The process of embodiment 295, wherein steps (a) and (b) are repeated three times.

299. The process of embodiment 295, wherein steps (a) and (b) are repeated four times.

300. The process of embodiment 295, wherein steps (a) and (b) are repeated five times.

301. The process of any one of embodiments 295 to 300, wherein the first temperature range is 30° C. to 45° C.

302. The process of embodiment 301, wherein the temperature in the first temperature range is 37° C.

303. The process of any one of embodiments 295 to 302, wherein the second temperature range is 50° C. to 65° C.

304. The process of embodiment 303, wherein the temperature in the second temperature range is 55° C.

305. The process of any one of embodiments 294 to 303, which comprises thermal cycling the mixture between 37° C. and 55° C.

306. A composition produced by a method comprising the process of any one of embodiments 294 to 305, optionally wherein the method further comprises a step of combining the product of the process with one or more buffers, preservatives, excipients, diluents, or a combination thereof.

307. A lipid binding protein-based complex for use in the treatment of an eye disease in a subject, optionally complexed with one or more ophthalmic drugs, wherein the administered amount of lipid binding protein-based complex is effective to reduce the severity of the eye disease.

308. The lipid binding protein-based complex for use of embodiment 307, wherein the eye disease is a disease associated with lipid accumulation; preferably the eye disease is selected from eye diseases associated with LCAT deficiency such as fish-eye disease; dry eye disease, such as dry eye disease associated with Meibomian gland dysfunction (MGD) or lacrimal gland dysfunction; blepharitis; an inflammatory eye disease such as uveitis; diseases of the cornea such as lipid keratopathy; macular edema; macular degeneration; retinal detachment; an ocular tumor; a fungal infection; a viral infection; a bacterial infection; multifocal choroiditis; diabetic retinopathy; proliferative vitreoretinopathy (PVR); sympathetic ophthalmia; Vogt Koyanagi-Harada (VKH) syndrome; histoplasmosis; uveal diffusion; vascular occlusion; and endophthalmitis.

309. The lipid binding protein-based complex for use of any one of embodiments 307 or 308, wherein the eye disease is fish-eye disease and the subject is homozygous or heterozygous for an LCAT mutation.

310. The lipid binding protein-based complex for use of any one of embodiments 307 to 309, wherein the lipid binding protein-based complex is a reconstituted HDL, HDL mimetic, a Cargomer or an Apomer; preferably the lipid binding protein-based complex selected from CER-001, CSL-111, CSL-112, CER-522 or ETC-216; more preferably the lipid binding protein-based complex is CER-001.

311. The lipid binding protein-based complex for use of any one of embodiments 307 to 310, wherein the lipid binding protein-based complex comprises one or more ophthalmic drugs complexed to the lipid binding protein-based complex, and wherein the one or more ophthalmic drugs comprise a steroid, a kinase inhibitor, an angiotensin II receptor antagonist, an aldose reductase inhibitor, an immunosuppressant, a carbonic anhydrase inhibitor, an antimicrobial agent, an antiviral agent, an antihistamine, an anti-inflammatory, or a combination thereof.

312. The lipid binding protein-based complex for use of any one of embodiments 307 to 311, wherein the one or more ophthalmic drugs comprise azithromycin, spironolactone, dexamethasone, difluprednate, estradiol, fluocinolone, fluorometholone, hydrocortisone, loteprednol etabonate, prednisolone, triamcinolone, rimexolone, axitinib, BMS-794833 (N-(4-((2-amino-3-chloropyridin-4-yl)oxy)-3-fluorophenyl)-5-(4-fluorophenyl)-4-oxo-1,4-dihydropyridine-3-carboxamide), carbozantinib, cediranib, dovitinib, lapatinib, lenvatinib, motesanib, nintedanib, orantinib, PD173074 (N-[2-[[4-(Diethylamino)butyl]amino]-6-(3,5-dimethoxyphenyl) pyri-do[2,3-d]pyrimidin-7-yl]-N′-(1,1-dimethylethyl) urea), pazopanib, regorafenib, sorafenib, tofacitinib, ZM323881 (5-((7-Benzyloxyquinazolin-4-yl)amino)-4-fluoro-2-methylphenol), candesartan, irbesartan, losartan, olmesartan, telmisartan, valsartan, 2-methylsorbino, sirolimus, acetazolamide, brinzolamide, dorzolamide, ethoxzolamide, diclofenac, indomethacin, nepafenac, latanoprost, travaprost, bimatoprost, tafluprost, dexamethasone palmitate, cyclosporine or a combination thereof.

313. The lipid binding protein-based complex for use of any one of embodiments 307 to 312, wherein the lipid binding protein-based complex is administered according to a dosing regimen which comprises:
(a) an induction regimen; and/or
(b) a consolidation regimen; and/or
(c) a maintenance regimen.

314. The lipid binding protein-based complex for use of embodiment 313, wherein the dosing regimen comprises an induction regimen which comprises administering multiple doses of the lipid binding protein-based complex to the subject, in which multiple doses are separated by 1 or more days.

315. The lipid binding protein-based complex for use of embodiment 314, wherein the doses following the initial dose of the induction regimen are separated by no more than 3 days, preferably the doses following the initial dose of the induction regimen are separated by one to three days.

316. The lipid binding protein-based complex for use of any one of embodiments 313 to 315, wherein the induction regimen is for a duration of at least one week, preferably the induction regimen is for a duration of two weeks, more preferably for a duration of three weeks.

317. The lipid binding protein-based complex for use of any one of embodiments 313 to 316, in which the induction regimen comprises administering to the subject three doses of the lipid binding protein-based complex per week.

318. The lipid binding protein-based complex for use of any one of embodiments 313 to 317, wherein the induction regimen comprises administering at least three doses of the lipid binding protein-based complex to the subject; preferably four or more doses, five or more doses, six or more doses, seven or more doses, eight or more doses, or nine or more doses; more preferably the induction regimen comprises administering nine or more doses of the lipid binding protein-based complex to the subject.

319. The lipid binding protein-based complex for use of any one of embodiments 313 to 318, wherein the induction regimen comprises administering the first dose of the lipid binding protein-based complex to the subject on day 1 and administering subsequent doses of the induction regimen to the subject on days 2, 4, 7, 9, 11, 14, 16, and 18.

320. The lipid binding protein-based complex for use of any one of embodiments 313 to 319, wherein the dosing regimen comprises a consolidation regimen which comprises administering multiple doses of the lipid binding protein-based complex to the subject, in which multiple doses are separated by 2 or more days.

321. The lipid binding protein-based complex for use of any one of embodiments 313 to 320, wherein the doses of the consolidation regimen are separated by no more than four days, preferably the doses of the consolidation regimen are separated from one another by three or four days.

322. The lipid binding protein-based complex for use of any one of embodiments 313 to 321, wherein the consolidation regimen is for a duration of at least three weeks.

323. The lipid binding protein-based complex for use of any one of embodiments 313 to 322, wherein the consolidation regimen comprises administering at least two doses of the lipid binding protein-based complex to the subject per week.

324. The lipid binding protein-based complex for use of any one of embodiments 313 to 323, wherein the consolidation regimen comprises administering at least two doses of the lipid binding protein-based complex to the subject; preferably three or more doses, four or more dose, five or more doses, or six or more doses; more preferably the consolidation regimen comprises administering six or more doses of the lipid binding protein-based complex to the subject.

325. The lipid binding protein-based complex for use of any one of embodiments 313 to 324, wherein the consolidation regimen comprises administering the six doses of the lipid binding protein-based complex to the subject on days 21, 24, 28, 31, 35, and 38 following an induction regimen which begins on day 1.

326. The lipid binding protein-based complex for use of any one of embodiments 313 to 325, wherein the dosing regimen comprises a maintenance regimen which comprises administering a dose of the lipid binding protein-based complex to the subject once every 3 or more days, preferably once every 5 or more days, more preferably one dose per week.

327. The lipid binding protein-based complex for use of any one of embodiments 313 to 326, wherein the maintenance regimen comprises administering the lipid binding protein-based complex to the subject for at least one month.

328. The lipid binding protein-based complex for use of any one of embodiments 313 to 327, wherein the dose of the lipid binding protein-based complex administered in the induction regimen, in the consolidation regimen and/or in the maintenance regimen is 4 to 30 mg/kg (on a protein weight basis); preferably 5 to 15 mg/kg (on a protein weight basis), 10 to 20 mg/kg (on a protein weight basis), or 15 to 25 mg/kg (on a protein weight basis).

329. The lipid binding protein-based complex for use of any one of embodiments 313 to 328, wherein the dose of the lipid binding protein-based complex administered in the induction regimen, in the consolidation regimen and/or in the maintenance regimen is 8 mg/kg (on a protein weight basis) or 10 mg/kg (on a protein weight basis).

330. The lipid binding protein-based complex for use of any one of embodiments 313 to 329, wherein the dose of the lipid binding protein-based complex administered in the induction regimen, in the consolidation regimen and/or in the maintenance regimen is 300 mg to 3000 mg; preferably 300 mg to 1500 mg, 400 mg to 1500 mg, 500 mg to 1200 mg, or 500 mg to 1000 mg.

331. The lipid binding protein-based complex for use of any one of embodiments 307 to 330, wherein the lipid binding protein-based complex is administered peripherally, optionally by infusion.

332. The lipid binding protein-based complex for use of any one of embodiments 307 to 330, wherein the lipid binding protein-based complex is administered locally, for example intraocularly, for example by intraocular injection (e.g., suprachoroidal injection, or suprascleral injection), or topically, for example by eye drop, or via an implant (e.g., a biodegradable or non-biodegradable disc, sheet, plug, rod, or pellet).

333. The lipid binding protein-based complex for use of any one of embodiments 307 to 332, wherein an antihistamine is administered prior to administration of one or more of the lipid binding protein-based complex doses.

334. The lipid binding protein-based complex for use of any one of embodiments 307 to 333, wherein the subject is also treated with a lipid control medication; preferably the lipid control medication comprises a statin such as atorvastatin, rosuvastatin, simvastatin, fluvastatin, lovastatin, or pravastatin; a cholesterol absorption inhibitor such as ezetimibe; niacin; aspirin; a proprotein convertase subtilisin/kexin type 9 (PCSK9) inhibitor such as an antibody selected from alirocumab, bococizumabevolocumab, 1D05-IgG2 and LY3015014, or a RNAi therapeutic such as ALN-PCSSC.

335. The lipid binding protein-based complex for use of any one of embodiments 307 to 334, wherein the subject is also treated with a standard of care therapy for the eye disease.

336. The lipid binding protein-based complex for use of any one of embodiments 307 to 335, wherein the lipid binding protein-based complex does not comprise and is not administered with a cell-penetrating peptide.

337. The lipid binding protein-based complex for use of any one of embodiments 307 to 336, wherein the lipid binding protein-based complex does not comprise and is not administered with a chemical penetration enhancer.

338. The lipid binding protein-based complex for use of any one of embodiments 307 to 337, wherein the lipid binding protein-based complex does not comprise and is not administered with a cytophilic peptide.

339. A lipoprotein complex for use in the treatment of an eye disease in a subject in need thereof,
wherein the lipoprotein complex comprises an ApoA-I apolipoprotein fraction and a lipid fraction which includes one or more phospholipids, and
wherein the subject has ocular lipid deposits.

340. The lipoprotein complex for use according to embodiment 339, wherein the lipoprotein complex comprises an ApoA-I apolipoprotein fraction and a lipid faction comprising at least one neutral phospholipid and, optionally, one or more negatively charged phospholipids.

341. The lipoprotein complex for use according to embodiment 339 or embodiment 340, wherein the lipoprotein complex is selected from CER-001, CSL-111 and ETC-216.

342. The lipoprotein complex for use according to any one of embodiments 339 to 341, wherein the lipoprotein complex comprises an ApoA-I apolipoprotein fraction and a lipoprotein faction comprising sphingomyelin and one or more negatively charged phospholipids.

343. The lipoprotein complex for use according to any one of embodiments 339 to 342, wherein the lipoprotein complex comprises an ApoA-I apolipoprotein fraction and a lipid fraction, wherein the lipid fraction consists essentially of sphingomyelin and about 0.2 to 6 wt % of a negatively charged phospholipid, and wherein the molar ratio of the lipid fraction to the ApoA-I apolipoprotein fraction is ranging from about 2:1 to 200:1.

344. The lipoprotein complex for use according to any one of embodiments 340 to 343, wherein the negatively charged phospholipids comprises 1,2-dipalmitoyl-sn-glycero-3-[phospho-rac-(1-glycerol)] (DPPG) or a salt thereof.

345. The lipoprotein complex for use according to any one of embodiments 339 to 344, wherein the lipoprotein complex is CER-001.

346. The lipoprotein complex for use according to any one of embodiments 339 to 345, wherein the ocular lipid deposits are corneal lipid deposits, retinal lipid deposits, palpebral lipid deposits or a combination thereof.

347. The lipoprotein complex for use according to any one of embodiments 339 to 346, wherein the eye disease is selected from dry eye associated with lipid accumulation including dry eye associated with Meibomian gland dysfunction (MGD) and dry eye associated with lacrimal gland dysfunction, blepharitis, inflammatory eye disease, uveitis including anterior uveitis, intermediate uveitis, posterior uveitis and panuveitis, diseases of the cornea including lipid keratopathy, eye diseases associated with LCAT deficiency including fish-eye disease, dry macular degeneration (dry AMD), Stargardt disease and Leber's idiopathic stellate neuroretinitis, macular edema, macular degeneration, retinal detachment, an ocular tumor, a fungal infection, a viral infection, a bacterial infection including bacterial conjunctivitis and trachoma, multifocal choroiditis, diabetic retinopathy, proliferative vitreoretinopathy (PVR), sympathetic ophthalmia, Vogt Koyanagi-Harada (VKH) syndrome, histoplasmosis, uveal diffusion, vascular occlusion, endophthalmitis, and glaucoma.

348. The lipoprotein complex for use according to any one of embodiments 339 to 347, wherein the administered amount of lipoprotein complex is effective to reduce ocular lipid deposits for the subject.

349. The lipoprotein complex for use according to any one of embodiments 339 to 348, wherein the lipoprotein complex further comprises one or more ophthalmic drugs.

350. The lipoprotein complex for use according to embodiment 349, wherein the ophthalmic drug is a steroid, a kinase inhibitor, an angiotensin II receptor antagonist, an aldose reductase inhibitor, an immunosuppressant, a carbonic anhydrase inhibitor, an antimicrobial agent, an antiviral agent, an antihistamine, an anti-inflammatory, a prostaglandin analog, or a combination thereof; preferably the ophthalmic drug is selected from azithromycin, dexamethasone, dexamethasone palmitate, difluprednate, estradiol, fluocinolone, fluorometholone, hydrocortisone, loteprednol etabonate, prednisolone, triamcinolone, rimexolone, spironolactone, axitinib, BMS-794833 (N-(4-((2-amino-3-chloropyridin-4-yl)oxy)-3-fluorophenyl)-5-(4-fluorophenyl)-4-oxo-1,4-dihydropyridine-3-carboxamide), carbozantinib, cediranib, dovitinib, lapatinib, lenvatinib, motesanib, nintedanib, orantinib, PD173074 (N-[2-[[4-(Diethylamino)butyl]amino]-6-(3,5-dimethoxyphenyl) pyri-do[2,3-d]pyrimidin-7-yl]-N′-(1,1-dimethylethyl) urea), pazopanib, regorafenib, sorafenib, tofacitinib, ZM323881 (5-((7-Benzyloxyquinazolin-4-yl)amino)-4-fluoro-2-methylphenol), candesartan, irbesartan, losartan, olmesartan, telmisartan, valsartan, 2-methylsorbino, sirolimus, cyclosporine, tacrolimus, acetazolamide, brinzolamide, dorzolamide, ethoxzolamide, methazolamide, acyclovir, chloramphenicol, chlortetracycline, ciprofloxacin, fusidic acid, gancyclovir, norfloxacin, ofloxacin, tetracycline, zidovudine, levocabastine, bromfenac, diclofenac, indomethacin, nepafenac, latanoprost, travaprost, bimatoprost, or a combination thereof.

351. The lipoprotein complex for use according to any one of embodiments 339 to 350, wherein the lipoprotein complex is administered peripherally, optionally by infusion.

352. The lipoprotein complex for use according to any one of embodiments 339 to 350, wherein the lipoprotein complex is administered intraocularly, preferably by intraocular injection, more preferably by intravitreal injection, suprachoroidal injection, or suprascleral injection.

353. The lipoprotein complex for use according to any one of embodiments 339 to 350, wherein the lipoprotein complex is administered by topical route, preferably using eye drops.

354. The lipoprotein complex for use according to any one of embodiments 339 to 353, wherein the lipoprotein complex does not comprise and is not administered with a cell-penetrating peptide.

355. The lipoprotein complex for use according to any one of embodiments 339 to 354, wherein the lipoprotein complex does not comprise and is not administered with a chemical penetration enhancer.

356. The lipoprotein complex for use according to any one of embodiments 339 to 355, wherein the lipoprotein complex does not comprise and is not administered with a cytophilic peptide.

357. A method of treating an eye disease in a subject, comprising:
(a) administering to the subject an Apolipoprotein A-I (“ApoA-I”) formulation comprising ApoA-I and one or more lipids, wherein the ApoA-I and the lipids are in the form of lipoprotein complexes, wherein:
   (i) the subject has a cataract, uveitis, an eye disease associated with lipid accumulation, lipid keratopathy, corneal dystrophy, corneal opacity, dry eye disease, blepharitis, an inflammatory eye disease, macular edema, macular degeneration, retinal detachment, an ocular tumor, a fungal infection, a viral infection, a bacterial infection (e.g., bacterial conjunctivitis or trachoma), multifocal choroiditis, diabetic retinopathy, proliferative vitreoretinopathy (PVR), sympathetic ophthalmia, Vogt Koyanagi-Harada (VKH) syndrome, histoplasmosis, uveal diffusion, vascular occlusion, endophthalmitis, glaucoma, Stargardt disease, ocular lipid deposits; and/or
   (ii) the formulation is administered by suprachoroidal injection, suprascleral injection, an implant, iontophoresis, or electroporation.

358. The method of embodiment 357, wherein the ApoA-I has the amino acid sequence of amino acids 25-267 of SEQ ID NO:2.

359. The method of embodiment 357 or embodiment 358, wherein the ApoA-I is recombinantly expressed.

360. The method of any one of embodiments 357 to 359, wherein said one or more lipids comprise neutral lipids.

361. The method of embodiment 360, wherein the neutral lipids comprise sphingomyelin.

362. The method of embodiment 361, wherein the neutral lipids consist of sphingomyelin.

363. The method of embodiment 361 or embodiment 362, wherein the sphingomyelin comprises natural sphingomyelin.

364. The method of embodiment 363, wherein the natural sphingomyelin is chicken egg sphingomyelin.

365. The method of embodiment 361 or embodiment 362, wherein the sphingomyelin comprises synthetic sphingomyelin.

366. The method of embodiment 365, wherein the synthetic sphingomyelin is palmitoylsphingomyelin.

367. The method of any one of embodiments 357 to 362, wherein the one or more lipids further comprise negatively charged lipids.

368. The method of embodiment 367, wherein the negatively charged lipids comprise 1,2-dipalmitoyl-sn-glycero-3-[phospho-rac-(1-glycerol)] (“DPPG”) or a salt thereof.

369. The method of embodiment 368, wherein the negatively charged lipids consist of 1,2-dipalmitoyl-sn-glycero-3-[phospho-rac-(1-glycerol)] (“DPPG”) or a salt thereof.

370. The method of any one of embodiments 363 to 369, wherein the molar ratio of the components of the negatively charged lipid to the neutral lipid to the ApoA-I in the formulation is 2-6:90-120:1.

371. The method of any one of embodiments 360 to 370, wherein said lipids consist of 95 to 99 weight % neutral phospholipid and 1 to 5 weight % negatively charged phospholipid.

372. The method embodiment 371, wherein said lipids consist of 96 to 98 weight % neutral phospholipid and 2 to 4 weight % negatively charged phospholipid.

373. The method of embodiment 372, wherein said lipids consists of 97 weight % neutral phospholipid and 3 weight % negatively charged phospholipid.

374. The method of any one of embodiments 357 to 373 which has an ApoA-I to lipid ratio ranging from 1:2 to 1:3 by weight.

375. The method of embodiment 374, which has an ApoA-I to lipid ratio of about 1:2.7 by weight.

376. The method of any one of embodiments 357 to 375, wherein the lipoprotein complexes are at least 95% homogeneous as reflected by a single peak in gel permeation chromatography.

377. The method of any one of embodiments 357 to 376, wherein the formulation is a carrier for or has complexed thereto one or more ophthalmic drugs, optionally wherein one or more of the one or more ophthalmic drugs are (i) hydrophobic and/or (ii) poorly water soluble or water insoluble.

378. The method of embodiment 377, wherein the one or more ophthalmic drugs comprise a steroid, a kinase inhibitor, an angiotensin II receptor antagonist, an aldose reductase inhibitor, an immunosuppressant, a carbonic anhydrase inhibitor, an antimicrobial agent, an antiviral agent, an antihistamine, an anti-inflammatory, a prostaglandin analog, or a combination thereof.

379. The method of embodiment 377 or embodiment 378, wherein the one or more ophthalmic drugs comprise azithromycin, dexamethasone, difluprednate, estradiol, fluocinolone, fluorometholone, hydrocortisone, loteprednol etabonate, prednisolone, triamcinolone, rimexolone, spironolactone, axitinib, BMS-794833 (N-(4-((2-amino-3-chloropyridin-4-yl)oxy)-3-fluorophenyl)-5-(4-fluorophenyl)-4-oxo-1,4-dihydropyridine-3-carboxamide), carbozantinib, cediranib, dovitinib, lapatinib, lenvatinib, motesanib, nintedanib, orantinib, PD173074 (N-[2-[[4-(Diethylamino)butyl]amino]-6-(3,5-dimethoxyphenyl) pyri-do[2,3-d]pyrimidin-7-yl]-N′-(1,1-dimethylethyl) urea), pazopanib, regorafenib, sorafenib, tofacitinib, ZM323881 (5-((7-Benzyloxyquinazolin-4-yl)amino)-4-fluoro-2-methylphenol), candesartan, irbesartan, losartan, olmesartan, telmisartan, valsartan, 2-methylsorbino, sirolimus, cyclosporine, tacrolimus, acetazolamide, brinzolamide, dorzolamide, ethoxzolamide, methazolamide, acyclovir, chloramphenicol, chlortetracycline, ciprofloxacin, fusidic acid, gancyclovir, norfloxacin, ofloxacin, tetracycline, zidovudine, levocabastine, bromfenac, diclofenac, indomethacin, nepafenac, latanoprost, travaprost, bimatoprost, dexamethasone palmitate, or a combination thereof.

380. The method of any one of embodiments 357 to 379, wherein the eye disease is fish-eye disease.

381. The method of any one of embodiments 357 to 379, wherein the eye disease is secondary lipid keratopathy, an inherited corneal dystrophy, an anterior or superficial corneal dystrophy, a stromal corneal dystrophy, or a posterior corneal dystrophy.

382. The method of any one of embodiments 357 to 379, wherein the subject has corneal opacity and the method comprises administering an amount of the lipid binding protein-based complex effective to reduce the opacity of the subject's cornea(s).

383. The method of any one of embodiments 357 to 379, wherein the eye disease is dry eye disease that is (a) associated with Meibomian gland dysfunction (MGD), optionally wherein the MGD is obstructive MGD or (b) associated with lacrimal gland dysfunction.

384. The method of any one of embodiments 357 to 379, wherein the eye disease is blepharitis an inflammatory eye disease, anterior uveitis, intermediate uveitis, posterior uveitis, or panuveitis.

385. The method of embodiment 384, wherein the eye disease is acute anterior uveitis.

386. The method of any one of embodiments 357 to 379, wherein the eye disease is dry macular degeneration.

387. The method of any one of embodiments 357 to 379, wherein the eye disease is wet macular degeneration.

388. The method of any one of embodiments 357 to 379, wherein the eye disease is diabetic macular edema.

389. The method of any one of embodiments 357 to 388, wherein the subject has impaired vision due to the eye disease and the method comprises administering an amount of the lipid binding protein-based complex to the subject which improves the subject's vision.

390. The method of any one of embodiments 357 to 389, wherein the subject has ocular lipid deposits, optionally wherein the ocular lipid deposits comprise corneal lipid deposits, retinal lipid deposits, palpebral lipid deposits or a combination thereof.

391. An Apolipoprotein A-I (“ApoA-I”) formulation comprising ApoA-I and one or more lipids, wherein the ApoA-I and the lipids are in the form of lipoprotein complexes, for treating an eye disease in a subject, wherein:
(i) the subject has a cataract, uveitis, an eye disease associated with lipid accumulation, lipid keratopathy, corneal dystrophy, corneal opacity, dry eye disease, blepharitis, an inflammatory eye disease, macular edema (e.g., diabetic macular edema), macular degeneration, retinal detachment, an ocular tumor, a fungal infection, a viral infection, a bacterial infection (e.g., bacterial conjunctivitis or trachoma), multifocal choroiditis, diabetic retinopathy, proliferative vitreoretinopathy (PVR), sympathetic ophthalmia, Vogt Koyanagi-Harada (VKH) syndrome, histoplasmosis, uveal diffusion, vascular occlusion, endophthalmitis, glaucoma, Stargardt disease, ocular lipid deposits; and/or
(ii) the formulation is administered by suprachoroidal injection, suprascleral injection, an implant, iontophoresis, or electroporation.

392. The formulation for use according to embodiment 391, wherein the ApoA-I has the amino acid sequence of amino acids 25-267 of SEQ ID NO:2.

393. The formulation for use according to embodiment 391 or embodiment 392, wherein the ApoA-I is recombinantly expressed.

394. The formulation of any one of embodiments 391 to 393, wherein said one or more lipids comprise neutral lipids.

395. The formulation of embodiment 394, wherein the neutral lipids comprise sphingomyelin.

396. The formulation of embodiment 395, wherein the neutral lipids consist of sphingomyelin.

397. The formulation for use according to embodiment 395 or embodiment 396, wherein the sphingomyelin comprises natural sphingomyelin.

398. The formulation for use according to embodiment 397, wherein the natural sphingomyelin is chicken egg sphingomyelin.

399. The formulation for use according to embodiment 395 or embodiment 396, wherein the sphingomyelin comprises synthetic sphingomyelin.

400. The formulation for use according to embodiment 399, wherein the synthetic sphingomyelin is palmitoylsphingomyelin.

401. The formulation for use according to any one of embodiments 391 to 396, wherein the one or more lipids further comprise negatively charged lipids.

402. The formulation for use according to embodiment 401, wherein the negatively charged lipids comprise 1,2-dipalmitoyl-sn-glycero-3-[phospho-rac-(1-glycerol)] (“DPPG”) or a salt thereof.

403. The formulation for use according to embodiment 402, wherein the negatively charged lipids consist of 1,2-dipalmitoyl-sn-glycero-3-[phospho-rac-(1-glycerol)] (“DPPG”) or a salt thereof.

404. The formulation for use according to any one of embodiments 397 to 403, wherein the molar ratio of the components of the negatively charged lipid to the neutral lipid to the ApoA-I in the formulation is 2-6:90-120:1.

405. The formulation for use according to any one of embodiments 394 to 404, wherein said lipids consist of 95 to 99 weight % neutral phospholipid and 1 to 5 weight % negatively charged phospholipid.

406. The method embodiment 405, wherein said lipids consist of 96 to 98 weight % neutral phospholipid and 2 to 4 weight % negatively charged phospholipid.

407. The formulation for use according to embodiment 406, wherein said lipids consists of 97 weight % neutral phospholipid and 3 weight % negatively charged phospholipid.

408. The formulation for use according to any one of embodiments 391 to 407 which has an ApoA-I to lipid ratio ranging from 1:2 to 1:3 by weight.

409. The formulation for use according to embodiment 408, which has an ApoA-I to lipid ratio of about 1:2.7 by weight.

410. The formulation for use according to any one of embodiments 391 to 409, wherein the lipoprotein complexes are at least 95% homogeneous as reflected by a single peak in gel permeation chromatography.

411. The formulation for use according to any one of embodiments 391 to 410, wherein the formulation is a carrier for or has complexed thereto one or more ophthalmic drugs, optionally wherein one or more of the one or more ophthalmic drugs are (i) hydrophobic and/or (ii) poorly water soluble or water insoluble.

412. The formulation for use according to embodiment 411, wherein the one or more ophthalmic drugs comprise a steroid, a kinase inhibitor, an angiotensin II receptor antagonist, an aldose reductase inhibitor, an immunosuppressant, a carbonic anhydrase inhibitor, an antimicrobial agent, an antiviral agent, an antihistamine, an anti-inflammatory, a prostaglandin analog, or a combination thereof.

413. The formulation for use according to embodiment 411 or embodiment 412, wherein the one or more ophthalmic drugs comprise azithromycin, dexamethasone, difluprednate, estradiol, fluocinolone, fluorometholone, hydrocortisone, loteprednol etabonate, prednisolone, triamcinolone, rimexolone, spironolactone, axitinib, BMS-794833 (N-(4-((2-amino-carboxamide), carbozantinib, cediranib, dovitinib, lapatinib, lenvatinib, motesanib, nintedanib, orantinib, PD173074 (N-[2-[[4-(Diethylamino)butyl]amino]-6-(3,5-dimethoxyphenyl) pyri-do[2,3-d]pyrimidin-7-yl]-N′-(1,1-dimethylethyl) urea), pazopanib, regorafenib, sorafenib, tofacitinib, ZM323881 (5-((7-Benzyloxyquinazolin-4-yl)amino)-4-fluoro-2-methylphenol), candesartan, irbesartan, losartan, olmesartan, telmisartan, valsartan, 2-methylsorbino, sirolimus, cyclosporine, tacrolimus, acetazolamide, brinzolamide, dorzolamide, ethoxzolamide, methazolamide, acyclovir, chloramphenicol, chlortetracycline, ciprofloxacin, fusidic acid, gancyclovir, norfloxacin, ofloxacin, tetracycline, zidovudine, levocabastine, bromfenac, diclofenac, indomethacin, nepafenac, latanoprost, travaprost, bimatoprost, dexamethasone palmitate, or a combination thereof.

414. The formulation for use according to any one of embodiments 391 to 413, wherein the eye disease is fish-eye disease.

415. The formulation for use according to any one of embodiments 391 to 413, wherein the eye disease is secondary lipid keratopathy, an inherited corneal dystrophy, an anterior or superficial corneal dystrophy, a stromal corneal dystrophy, or a posterior corneal dystrophy.

416. The formulation for use according to any one of embodiments 391 to 413, wherein the subject has corneal opacity and the method comprises administering an amount of the lipid binding protein-based complex effective to reduce the opacity of the subject's cornea(s).

417. The formulation for use according to any one of embodiments 391 to 413, wherein the eye disease is dry eye disease that is (a) associated with Meibomian gland dysfunction (MGD), optionally wherein the MGD is obstructive MGD or (b) associated with lacrimal gland dysfunction.

418. The formulation for use according to any one of embodiments 391 to 413, wherein the eye disease is blepharitis an inflammatory eye disease, anterior uveitis, intermediate uveitis, posterior uveitis, or panuveitis.

419. The formulation for use according to embodiment 418, wherein the eye disease is acute anterior uveitis.

420. The formulation for use according to any one of embodiments 391 to 413, wherein the eye disease is dry macular degeneration.

421. The formulation for use according to any one of embodiments 391 to 413, wherein the eye disease is wet macular degeneration.

422. The formulation for use according to any one of embodiments 391 to 413, wherein the eye disease is diabetic macular edema.

423. The formulation for use according to any one of embodiments 391 to 422, wherein the subject has impaired vision due to the eye disease and the method comprises administering an amount of the lipid binding protein-based complex to the subject which improves the subject's vision.

424. The formulation for use according to any one of embodiments 391 to 423, wherein the subject has ocular lipid deposits, optionally wherein the ocular lipid deposits comprise corneal lipid deposits, retinal lipid deposits, palpebral lipid deposits or a combination thereof.

425. An Apolipoprotein A-I (“ApoA-I”) formulation comprising ApoA-I and one or more lipids, wherein the ApoA-I and the lipids are in the form of lipoprotein complexes, for the manufacture of a medicament for treating an eye disease in a subject, wherein:
(i) the subject has a cataract, uveitis, an eye disease associated with lipid accumulation, lipid keratopathy, corneal dystrophy, corneal opacity, dry eye disease, blepharitis, an inflammatory eye disease, macular edema (e.g., diabetic macular edema), macular degeneration, retinal detachment, an ocular tumor, a fungal infection, a viral infection, a bacterial infection (e.g., bacterial conjunctivitis or trachoma), multifocal choroiditis, diabetic retinopathy, proliferative vitreoretinopathy (PVR), sympathetic ophthalmia, Vogt Koyanagi-Harada (VKH) syndrome, histoplasmosis, uveal diffusion, vascular occlusion, endophthalmitis, glaucoma, Stargardt disease, ocular lipid deposits; and/or
(ii) the formulation is administered by suprachoroidal injection, suprascleral injection, an implant, iontophoresis, or electroporation.

426. The formulation for use according to embodiment 425, wherein the ApoA-I has the amino acid sequence of amino acids 25-267 of SEQ ID NO:2.

427. The formulation for use according to embodiment 425 or embodiment 426, wherein the ApoA-I is recombinantly expressed.

428. The formulation of any one of embodiments 425 to 427, wherein said one or more lipids comprise neutral lipids.

429. The formulation of embodiment 428, wherein the neutral lipids comprise sphingomyelin.

430. The formulation of embodiment 429, wherein the neutral lipids consist of sphingomyelin.

431. The formulation for use according to embodiment 429 or embodiment 430, wherein the sphingomyelin comprises natural sphingomyelin.

432. The formulation for use according to embodiment 431, wherein the natural sphingomyelin is chicken egg sphingomyelin.

433. The formulation for use according to embodiment 429 or embodiment 430, wherein the sphingomyelin comprises synthetic sphingomyelin.

434. The formulation for use according to embodiment 433, wherein the synthetic sphingomyelin is palmitoylsphingomyelin.

435. The formulation for use according to any one of embodiments 425 to 430, wherein the one or more lipids further comprise negatively charged lipids.

436. The formulation for use according to embodiment 435, wherein the negatively charged lipids comprise 1,2-dipalmitoyl-sn-glycero-3-[phospho-rac-(1-glycerol)] (“DPPG”) or a salt thereof.

437. The formulation for use according to embodiment 436, wherein the negatively charged lipids consist of 1,2-dipalmitoyl-sn-glycero-3-[phospho-rac-(1-glycerol)] (“DPPG”) or a salt thereof.

438. The formulation for use according to any one of embodiments 431 to 437, wherein the molar ratio of the components of the negatively charged lipid to the neutral lipid to the ApoA-I in the formulation is 2-6:90-120:1.

439. The formulation for use according to any one of embodiments 428 to 438, wherein said lipids consist of 95 to 99 weight % neutral phospholipid and 1 to 5 weight % negatively charged phospholipid.

440. The method embodiment 439, wherein said lipids consist of 96 to 98 weight % neutral phospholipid and 2 to 4 weight % negatively charged phospholipid.

441. The formulation for use according to embodiment 440, wherein said lipids consists of 97 weight % neutral phospholipid and 3 weight % negatively charged phospholipid.

442. The formulation for use according to any one of embodiments 425 to 441 which has an ApoA-I to lipid ratio ranging from 1:2 to 1:3 by weight.

443. The formulation for use according to embodiment 442, which has an ApoA-I to lipid ratio of about 1:2.7 by weight.

444. The formulation for use according to any one of embodiments 425 to 443, wherein the lipoprotein complexes are at least 95% homogeneous as reflected by a single peak in gel permeation chromatography.

445. The formulation for use according to any one of embodiments 425 to 444, wherein the formulation is a carrier for or has complexed thereto one or more ophthalmic drugs, optionally wherein one or more of the one or more ophthalmic drugs are (i) hydrophobic and/or (ii) poorly water soluble or water insoluble.

446. The formulation for use according to embodiment 445, wherein the one or more ophthalmic drugs comprise a steroid, a kinase inhibitor, an angiotensin II receptor antagonist, an aldose reductase inhibitor, an immunosuppressant, a carbonic anhydrase inhibitor, an antimicrobial agent, an antiviral agent, an antihistamine, an anti-inflammatory, a prostaglandin analog, or a combination thereof.

447. The formulation for use according to embodiment 445 or embodiment 446, wherein the one or more ophthalmic drugs comprise azithromycin, dexamethasone, difluprednate, estradiol, fluocinolone, fluorometholone, hydrocortisone, loteprednol etabonate, prednisolone, triamcinolone, rimexolone, spironolactone, axitinib, BMS-794833 (N-(4-((2-amino-3-chloropyridin-4-yl)oxy)-3-fluorophenyl)-5-(4-fluorophenyl)-4-oxo-1,4-dihydropyridine-3-carboxamide), carbozantinib, cediranib, dovitinib, lapatinib, lenvatinib, motesanib, nintedanib, orantinib, PD173074 (N-[2-[[4-(Diethylamino)butyl]amino]-6-(3,5-dimethoxyphenyl) pyri-do[2,3-d]pyrimidin-7-yl]-N′-(1,1-dimethylethyl) urea), pazopanib, regorafenib, sorafenib, tofacitinib, ZM323881 (5-((7-Benzyloxyquinazolin-4-yl)amino)-4-fluoro-2-methylphenol), candesartan, irbesartan, losartan, olmesartan, telmisartan, valsartan, 2-methylsorbino, sirolimus, cyclosporine, tacrolimus, acetazolamide, brinzolamide, dorzolamide, ethoxzolamide, methazolamide, acyclovir, chloramphenicol, chlortetracycline, ciprofloxacin, fusidic acid, gancyclovir, norfloxacin, ofloxacin, tetracycline, zidovudine, levocabastine, bromfenac, diclofenac, indomethacin, nepafenac, latanoprost, travaprost, bimatoprost, dexamethasone palmitate, or a combination thereof.

448. The formulation for use according to any one of embodiments 425 to 447, wherein the eye disease is fish-eye disease.

449. The formulation for use according to any one of embodiments 425 to 447, wherein the eye disease is secondary lipid keratopathy, an inherited corneal dystrophy, an anterior or superficial corneal dystrophy, a stromal corneal dystrophy, or a posterior corneal dystrophy.

450. The formulation for use according to any one of embodiments 425 to 447, wherein the subject has corneal opacity and the method comprises administering an amount of the lipid binding protein-based complex effective to reduce the opacity of the subject's cornea(s).

451. The formulation for use according to any one of embodiments 425 to 447, wherein the eye disease is dry eye disease that is (a) associated with Meibomian gland dysfunction (MGD), optionally wherein the MGD is obstructive MGD or (b) associated with lacrimal gland dysfunction.

452. The formulation for use according to any one of embodiments 425 to 447, wherein the eye disease is blepharitis an inflammatory eye disease, anterior uveitis, intermediate uveitis, posterior uveitis, or panuveitis.

453. The formulation for use according to embodiment 452, wherein the eye disease is acute anterior uveitis.

454. The formulation for use according to any one of embodiments 425 to 447, wherein the eye disease is dry macular degeneration.

455. The formulation for use according to any one of embodiments 425 to 447, wherein the eye disease is wet macular degeneration.

456. The formulation for use according to any one of embodiments 425 to 447, wherein the eye disease is diabetic macular edema.

457. The formulation for use according to any one of embodiments 425 to 456, wherein the subject has impaired vision due to the eye disease and the method comprises administering an amount of the lipid binding protein-based complex to the subject which improves the subject's vision.

458. The formulation for use according to any one of embodiments 425 to 457, wherein the subject has ocular lipid deposits, optionally wherein the ocular lipid deposits comprise corneal lipid deposits, retinal lipid deposits, palpebral lipid deposits or a combination thereof.

Various aspects of the present disclosure are described in the embodiments set forth in the following numbered paragraphs of Group 2.

### Group 2:

1. A lipid binding protein-based complex for use in a method of treating an eye disease in a subject, wherein the lipid binding protein-based complex (a) is CER-001 and/or (b) is a carrier for one or more ophthalmic drugs, optionally wherein:
(a) the subject has a cataract or acute anterior uveitis; and/or
(b) the lipid binding protein-based complex is administered by suprachoroidal injection, suprascleral injection, an implant, iontophoresis, or electroporation.

2. The lipid binding protein-based complex for use according to embodiment 1, wherein the eye disease is a disease associated with lipid accumulation.

3. The lipid binding protein-based complex for use according to embodiment 2, wherein the eye disease is fish-eye disease.

4. The lipid binding protein-based complex for use according to embodiment 2, wherein the eye disease is lipid keratopathy, optionally wherein the lipid keratopathy is secondary lipid keratopathy.

5. The lipid binding protein-based complex for use according to embodiment 2, wherein the eye disease is corneal dystrophy, for example an inherited corneal dystrophy, an anterior or superficial corneal dystrophy, a stromal corneal dystrophy, or a posterior corneal dystrophy.

6 The lipid binding protein-based complex for use according to any one of embodiments 1 to 5, wherein the subject has corneal opacity and the method comprises administering an amount of the lipid binding protein-based complex effective to reduce the opacity of the subject's cornea(s).

7. The lipid binding protein-based complex for use according to embodiment 1 or embodiment 2, wherein the subject has a cataract.

8. The lipid binding protein-based complex for use according to embodiment 1 or embodiment 2, wherein the eye disease is dry eye disease, optionally wherein the dry eye disease is (a) associated with Meibomian gland dysfunction (MGD), optionally wherein the MGD is obstructive MGD or (b) associated with lacrimal gland dysfunction.

9. The lipid binding protein-based complex for use according to embodiment 1 or embodiment 2, wherein the eye disease is blepharitis.

10. The lipid binding protein-based complex for use according to embodiment 1 or embodiment 2, wherein the eye disease is an inflammatory eye disease.

11. The lipid binding protein-based complex for use according to embodiment 1 or embodiment 2, wherein the eye disease is uveitis, optionally wherein the uveitis is anterior uveitis, intermediate uveitis, posterior uveitis, or panuveitis.

12. The lipid binding protein-based complex for use according to embodiment 11, wherein the eye disease is acute anterior uveitis.

13. The lipid binding protein-based complex for use according to embodiment 1 or embodiment 2, wherein the eye disease is macular edema, macular degeneration, retinal detachment, an ocular tumor, a fungal infection, a viral infection, a bacterial infection (e.g., bacterial conjunctivitis or trachoma), multifocal choroiditis, diabetic retinopathy, proliferative vitreoretinopathy (PVR), sympathetic ophthalmia, Vogt Koyanagi-Harada (VKH) syndrome, histoplasmosis, uveal diffusion, vascular occlusion, endophthalmitis, or glaucoma.

14. The lipid binding protein-based complex for use according to embodiment 1 or embodiment 2, wherein the eye disease is dry macular degeneration.

15. The lipid binding protein-based complex for use according to embodiment 1 or embodiment 2, wherein the eye disease is wet macular degeneration.

16. The lipid binding protein-based complex for use according to embodiment 1 or embodiment 2, wherein the eye disease is Stargardt disease.

17. The lipid binding protein-based complex for use according to embodiment 1 or embodiment 2, wherein the eye disease is diabetic retinopathy, optionally wherein the subject has diabetic macular edema.

18. The lipid binding protein-based complex for use according to any one of embodiments 1 to 17, wherein the subject has impaired vision due to the eye disease and the method comprises administering an amount of the lipid binding protein-based complex to the subject which improves the subject's vision.

19. The lipid binding protein-based complex for use according to any one of embodiments 1 to 18, wherein the subject has ocular lipid deposits, optionally wherein the ocular lipid deposits comprise corneal lipid deposits, retinal lipid deposits, palpebral lipid deposits or a combination thereof.

20. The lipid binding protein-based complex for use according to any one of embodiments 1 to 19, wherein the lipid binding protein-based complex is CER-001.

21. The lipid binding protein-based complex for use according to any one of embodiments 1 to 20, wherein the lipid binding protein-based complex is a carrier for one or more ophthalmic drugs, optionally wherein (I) the lipid binding protein-based complex is CER-001, CSL-111, CSL-112, ETC-216, CER-522, delipidated HDL, an Apomer, or a Cargomer and/or (II) the one or more ophthalmic drugs are (i) hydrophobic and/or (ii) poorly water soluble or water insoluble.

22. The lipid binding protein-based complex for use according to embodiment 21, wherein the one or more ophthalmic drugs comprise a steroid, a kinase inhibitor, an angiotensin II receptor antagonist, an aldose reductase inhibitor, an immunosuppressant, a carbonic anhydrase inhibitor, an antimicrobial agent, an antiviral agent, an antihistamine, an anti-inflammatory, a prostaglandin analog, or a combination thereof.

23. The lipid binding protein-based complex for use according to embodiment 21 or embodiment 22, wherein the one or more ophthalmic drugs comprise dexamethasone palmitate, azithromycin, dexamethasone, difluprednate, estradiol, fluocinolone, fluorometholone, hydrocortisone, loteprednol etabonate, prednisolone, triamcinolone, rimexolone, spironolactone, axitinib, BMS-794833 (N-(4-((2-amino-3-chloropyridin-4-yl)oxy)-3-fluorophenyl)-5-(4-fluorophenyl)-4-oxo-1,4-dihydropyridine-3-carboxamide), carbozantinib, cediranib, dovitinib, lapatinib, lenvatinib, motesanib, nintedanib, orantinib, PD173074 (N-[2-[[4-(Diethylamino)butyl]amino]-6-(3,5-dimethoxyphenyl) pyri-do[2,3-d]pyrimidin-7-yl]-N′-(1,1-dimethylethyl) urea), pazopanib, regorafenib, sorafenib, tofacitinib, ZM323881 (5-((7-Benzyloxyquinazolin-4-yl)amino)-4-fluoro-2-methylphenol), candesartan, irbesartan, losartan, olmesartan, telmisartan, valsartan, 2-methylsorbino, sirolimus, cyclosporine, tacrolimus, acetazolamide, brinzolamide, dorzolamide, ethoxzolamide, methazolamide, acyclovir, chloramphenicol, chlortetracycline, ciprofloxacin, fusidic acid, gancyclovir, norfloxacin, ofloxacin, tetracycline, zidovudine, levocabastine, bromfenac, diclofenac, indomethacin, nepafenac, latanoprost, travaprost, bimatoprost, or a combination thereof.

24. The lipid binding protein-based complex for use according to any one of embodiments 21 to 23, wherein the one or more ophthalmic drugs comprise dexamethasone palmitate.

25. The lipid binding protein-based complex for use according to any one of embodiments 21 to 23, wherein the one or more ophthalmic drugs comprise dexamethasone.

26. The lipid binding protein-based complex for use according to any one of embodiments 21 to 23, wherein the one or more ophthalmic drugs comprise tacrolimus.

27. The lipid binding protein-based complex for use according to any one of embodiments 1 to 26, wherein method comprises administering the lipid binding protein-based complex peripherally, optionally by infusion.

28. The lipid binding protein-based complex for use according to embodiment 27, wherein the method comprises administering the lipid binding protein-based complex according to a dosing regimen which comprises:
(a) an induction regimen; and/or
(b) a consolidation regimen; and/or
(c) a maintenance regimen,
optionally wherein the lipid binding protein-based complex is CER-001.

29. The lipid binding protein-based complex for use according to any one of embodiments 1 to 26, wherein the method comprises administering the lipid binding protein-based complex locally.

30. The lipid binding protein-based complex for use according to embodiment 29, wherein method comprises administering the lipid binding protein-based complex intraocularly.

31. The lipid binding protein-based complex for use according to embodiment 30, wherein the method comprises administering the lipid binding protein-based complex by intraocular injection, optionally wherein the intraocular injection is suprachoroidal injection, suprascleral injection, intra-vitreal injection, sub-conjunctival injection, parabulbar injection, peribulbar injection, or retro-bulbar injection.

32. The lipid binding protein-based complex for use according to embodiment 29, wherein the method comprises administering the lipid binding protein-based complex via an implant, optionally wherein the implant is a biodegradable or non-biodegradable disc, sheet, plug, rod, or pellet.

33. The lipid binding protein-based complex for use according to embodiment 29, wherein the method comprises administering the lipid binding protein-based complex topically.

34. The lipid binding protein-based complex for use according to embodiment 33, wherein the lipid binding protein-based complex is formulated as an eye drop.

35. A process for making a composition comprising a lipid binding protein-based complex and one or more ophthalmic drugs, the process comprising thermal cycling a mixture comprising the lipid binding protein-based complex and the one or more ophthalmic drugs, optionally wherein (I) the lipid binding protein-based complex is CER-001, CSL-111, CSL-112, ETC-216, CER-522, delipidated HDL, an Apomer, or a Cargomer and/or (II) one or more of the one or more ophthalmic drugs are (i) hydrophobic and/or (ii) poorly water soluble or water insoluble.

36. The process of embodiment 35, wherein the thermal cycling comprises
(a) heating the mixture from a temperature in a first temperature range to a temperature in a second temperature range,
(b) cooling the mixture of (a) from a temperature in the second temperature range to a temperature in the first temperature range; and
(c) optionally repeating steps (a) and (b) at least once.

37. The process of embodiment 36, wherein steps (a) and (b) are repeated, one, two, three, four, or five times.

38. The process of embodiment 36 or embodiment 37, wherein the first temperature range is 30° C. to 45° C.

39. The process of embodiment 38, wherein the temperature in the first temperature range is 37° C.

40. The process of any one embodiments 36 to 39, wherein the second temperature range is 50° C. to 65° C.

41. The process of embodiment 40, wherein the temperature in the second temperature range is 55° C.

42. The process of any one of embodiments 35 to 41, which comprises thermal cycling the mixture between 37° C. and 55° C.

43. A composition produced by a method comprising the process of any one of embodiments 35 to 42, optionally wherein the composition is formulated as an eye drop.

44. A composition which is an implant, optionally wherein the implant is a biodegradable or non-biodegradable disc, sheet, plug, rod, or pellet, comprising a lipid binding protein-based complex and one or more ophthalmic drugs, optionally wherein (I) the lipid binding protein-based complex is CER-001, CSL-111, CSL-112, ETC-216, CER-522, delipidated HDL, an Apomer, or a Cargomer and/or (II) one or more of the one or more ophthalmic drugs are (i) hydrophobic and/or (ii) poorly water soluble or water insoluble.

45. The composition of embodiment 43 or embodiment 44, wherein the lipid binding protein-based complex is CER-001.

46. The composition of any one of embodiments 43 to 45, wherein the one or more ophthalmic drugs comprise a steroid, a kinase inhibitor, an angiotensin II receptor antagonist, an aldose reductase inhibitor, an immunosuppressant, a carbonic anhydrase inhibitor, an antimicrobial agent, an antiviral agent, an antihistamine, an anti-inflammatory, or a combination thereof.

47. The composition of any one of embodiments 43 to 46, wherein the one or more ophthalmic drugs comprise dexamethasone palmitate, azithromycin, dexamethasone, difluprednate, estradiol, fluocinolone, fluorometholone, hydrocortisone, loteprednol etabonate, prednisolone, triamcinolone, rimexolone, spironolactone, axitinib, BMS-794833 (N-(4-((2-amino-carboxamide), carbozantinib, cediranib, dovitinib, lapatinib, lenvatinib, motesanib, nintedanib, orantinib, PD173074 (N-[2-[[4-(Diethylamino)butyl]amino]-6-(3,5-dimethoxyphenyl) pyri-do[2,3-d]pyrimidin-7-yl]-N′-(1,1-dimethylethyl) urea), pazopanib, regorafenib, sorafenib, tofacitinib, ZM323881 (5-((7-Benzyloxyquinazolin-4-yl)amino)-4-fluoro-2-methylphenol), candesartan, irbesartan, losartan, olmesartan, telmisartan, valsartan, 2-methylsorbino, sirolimus, cyclosporine, tacrolimus, acetazolamide, brinzolamide, dorzolamide, ethoxzolamide, methazolamide, acyclovir, chloramphenicol, chlortetracycline, ciprofloxacin, fusidic acid, gancyclovir, norfloxacin, ofloxacin, tetracycline, zidovudine, levocabastine, bromfenac, diclofenac, indomethacin, nepafenac, or a combination thereof.

48. The composition of embodiment 47, wherein the one or more ophthalmic drugs comprise dexamethasone palmitate, optionally wherein the concentration of dexamethasone palmitate in the composition is 1 mg/ml.

49. The composition of embodiment 47, wherein the one or more ophthalmic drugs comprise dexamethasone.

50. The composition of embodiment 47, wherein the one or more ophthalmic drugs comprise ophthalmic drugs comprise tacrolimus.

51. The composition of any one of embodiments 43 to 50, which is a pharmaceutical composition further comprising one or more buffers, preservatives, excipients, diluents, or a combination thereof, optionally wherein the pharmaceutical composition is formulated as an eye drop.

52. A composition which is an implant comprising a lipid binding protein-based complex, optionally wherein the lipid binding protein-based complex is CER-001, optionally wherein the implant is a biodegradable or non-biodegradable disc, sheet, plug, rod, or pellet.

53. The composition of any one of embodiments 43 to 52 for use in a method of treating an eye disease in a subject.

While various specific embodiments have been illustrated and described, it will be appreciated that various changes can be made without departing from the spirit and scope of the disclosure(s)

### 9. INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

Any discussion of documents, acts, materials, devices, articles or the like that has been included in this specification is solely for the purpose of providing a context for the present disclosure. It is not to be taken as an admission that any or all of these matters form part of the prior art base or were common general knowledge in the field relevant to the present disclosure as it existed anywhere before the priority date of this application.

What is claimed is:

1. A lipid binding protein-based complex for use in a method of treating an eye disease in a subject, wherein the lipid binding protein-based complex (a) is CER-001 and/or (b) is a carrier for one or more ophthalmic drugs, wherein:
(a) the subject has a cataract or acute anterior uveitis; and/or
(b) the lipid binding protein-based complex is administered by suprachoroidal injection, suprascleral injection, an implant, iontophoresis, or electroporation.

2. The lipid binding protein-based complex for use according to claim 1, wherein the eye disease is a disease associated with lipid accumulation.

3. The lipid binding protein-based complex for use according to claim 2, wherein the eye disease is fish-eye disease.

4. The lipid binding protein-based complex for use according to claim 2, wherein the eye disease is lipid keratopathy, optionally wherein the lipid keratopathy is secondary lipid keratopathy.

5. The lipid binding protein-based complex for use according to claim 2, wherein the eye disease is corneal dystrophy, for example an inherited corneal dystrophy, an anterior or superficial corneal dystrophy, a stromal corneal dystrophy, or a posterior corneal dystrophy.

6. The lipid binding protein-based complex for use according to any one of claims 1 to 5, wherein the subject has corneal opacity and the method comprises administering an amount of the lipid binding protein-based complex effective to reduce the opacity of the subject's cornea(s).

7. The lipid binding protein-based complex for use according to claim 1 or claim 2, wherein the subject has a cataract.

8. The lipid binding protein-based complex for use according to claim 1 or claim 2, wherein the eye disease is dry eye disease, optionally wherein the dry eye disease is (a) associated with Meibomian gland dysfunction (MGD), optionally wherein the MGD is obstructive MGD or (b) associated with lacrimal gland dysfunction.

9. The lipid binding protein-based complex for use according to claim 1 or claim 2, wherein the eye disease is blepharitis.

10. The lipid binding protein-based complex for use according to claim 1 or claim 2, wherein the eye disease is an inflammatory eye disease.

11. The lipid binding protein-based complex for use according to claim 1 or claim 2, wherein the eye disease is uveitis, optionally wherein the uveitis is anterior uveitis, intermediate uveitis, posterior uveitis, or panuveitis.

12. The lipid binding protein-based complex for use according to claim 11, wherein the eye disease is acute anterior uveitis.

13. The lipid binding protein-based complex for use according to claim 1 or claim 2, wherein the eye disease is macular edema, macular degeneration, retinal detachment, an ocular tumor, a fungal infection, a viral infection, a bacterial infection (e.g., bacterial conjunctivitis or trachoma), multifocal choroiditis, diabetic retinopathy, proliferative vitreoretinopathy (PVR), sympathetic ophthalmia, Vogt Koyanagi-Harada (VKH) syndrome, histoplasmosis, uveal diffusion, vascular occlusion, endophthalmitis, or glaucoma.

14. The lipid binding protein-based complex for use according to claim 1 or claim 2, wherein the eye disease is dry macular degeneration.

15. The lipid binding protein-based complex for use according to claim 1 or claim 2, wherein the eye disease is wet macular degeneration.

16. The lipid binding protein-based complex for use according to claim 1 or claim 2, wherein the eye disease is Stargardt disease.

17. The lipid binding protein-based complex for use according to claim 1 or claim 2, wherein the eye disease is diabetic retinopathy, optionally wherein the subject has diabetic macular edema.

18. The lipid binding protein-based complex for use according to any one of claims 1 to 17, wherein the subject has impaired vision due to the eye disease and the method comprises administering an amount of the lipid binding protein-based complex to the subject which improves the subject's vision.

19. The lipid binding protein-based complex for use according to any one of claims 1 to 18, wherein the subject has ocular lipid deposits, optionally wherein the ocular lipid deposits comprise corneal lipid deposits, retinal lipid deposits, palpebral lipid deposits or a combination thereof.

20. The lipid binding protein-based complex for use according to any one of claims 1 to 19, wherein the lipid binding protein-based complex is CER-001.

21. The lipid binding protein-based complex for use according to any one of claims 1 to 20, wherein the lipid binding protein-based complex is a carrier for one or more ophthalmic drugs, optionally wherein (I) the lipid binding protein-based complex is CER-001, CSL-111, CSL-112, ETC-216, CER-522, delipidated HDL, an Apomer, or a Cargomer and/or (II) the one or more ophthalmic drugs are (i) hydrophobic and/or (ii) poorly water soluble or water insoluble.

22. The lipid binding protein-based complex for use according to claim 21, wherein the one or more ophthalmic drugs comprise a steroid, a kinase inhibitor, an angiotensin II receptor antagonist, an aldose reductase inhibitor, an immunosuppressant, a carbonic anhydrase inhibitor, an antimicrobial agent, an antiviral agent, an antihistamine, an anti-inflammatory, a prostaglandin analog, or a combination thereof.

23. The lipid binding protein-based complex for use according to claim 21 or claim 22, wherein the one or more ophthalmic drugs comprise dexamethasone palmitate, azithromycin, dexamethasone, difluprednate, estradiol, fluocinolone, fluorometholone, hydrocortisone, loteprednol etabonate, prednisolone, triamcinolone, rimexolone, spironolactone, axitinib, BMS-794833 (N-(4-((2-amino-3-chloropyridin-4-yl)oxy)-3-fluorophenyl)-5-(4-fluorophenyl)-4-oxo-1,4-dihydropyridine-3-carboxamide), carbozantinib, cediranib, dovitinib, lapatinib, lenvatinib, motesanib, nintedanib, orantinib, PD173074 (N-[2-[[4-(Diethylamino)butyl]amino]-6-(3,5-dimethoxyphenyl) pyri-do[2,3-d]pyrimidin-7-yl]-N′-(1,1-dimethylethyl) urea), pazopanib, regorafenib, sorafenib, tofacitinib, ZM323881 (5-((7-Benzyloxyquinazolin-4-yl)amino)-4-fluoro-2-methylphenol), candesartan, irbesartan, losartan, olmesartan, telmisartan, valsartan, 2-methylsorbino, sirolimus, cyclosporine, tacrolimus, acetazolamide, brinzolamide, dorzolamide, ethoxzolamide, methazolamide, acyclovir, chloramphenicol, chlortetracycline, ciprofloxacin, fusidic acid, gancyclovir, norfloxacin, ofloxacin, tetracycline, zidovudine, levocabastine, bromfenac, diclofenac, indomethacin, nepafenac, latanoprost, travaprost, bimatoprost, or a combination thereof.

24. The lipid binding protein-based complex for use according to any one of claims 21 to 23, wherein the one or more ophthalmic drugs comprise dexamethasone palmitate.

25. The lipid binding protein-based complex for use according to any one of claims 21 to 23, wherein the one or more ophthalmic drugs comprise dexamethasone.

26. The lipid binding protein-based complex for use according to any one of claims 21 to 23, wherein the one or more ophthalmic drugs comprise tacrolimus.

27. The lipid binding protein-based complex for use according to any one of claims 1 to 26, wherein method comprises administering the lipid binding protein-based complex peripherally, optionally by infusion.

28. The lipid binding protein-based complex for use according to claim 27, wherein the method comprises administering the lipid binding protein-based complex according to a dosing regimen which comprises:
(a) an induction regimen; and/or
(b) a consolidation regimen; and/or
(c) a maintenance regimen,
optionally wherein the lipid binding protein-based complex is CER-001.

29. The lipid binding protein-based complex for use according to any one of claims 1 to 26, wherein the method comprises administering the lipid binding protein-based complex locally.

30. The lipid binding protein-based complex for use according to claim 29, wherein method comprises administering the lipid binding protein-based complex intraocularly.

31. The lipid binding protein-based complex for use according to claim 30, wherein the method comprises administering the lipid binding protein-based complex by intraocular injection, optionally wherein the intraocular injection is suprachoroidal injection, suprascleral injection, intra-vitreal injection, sub-conjunctival injection, parabulbar injection, peribulbar injection, or retro-bulbar injection.

32. The lipid binding protein-based complex for use according to claim 29, wherein the method comprises administering the lipid binding protein-based complex via an implant, optionally wherein the implant is a biodegradable or non-biodegradable disc, sheet, plug, rod, or pellet.

33. The lipid binding protein-based complex for use according to claim 29, wherein the method comprises administering the lipid binding protein-based complex topically.

34. The lipid binding protein-based complex for use according to claim 33, wherein the lipid binding protein-based complex is formulated as an eye drop.

35. A composition which is an implant, optionally wherein the implant is a biodegradable or non-biodegradable disc, sheet, plug, rod, or pellet, comprising a lipid binding protein-based complex and one or more ophthalmic drugs, optionally wherein (1) the lipid binding protein-based complex is CER-001, CSL-111, CSL-112, ETC-216, CER-522, delipidated HDL, an Apomer, or a Cargomer and/or (II) one or more of the one or more ophthalmic drugs are (i) hydrophobic and/or (ii) poorly water soluble or water insoluble.

36. The composition of claim 35, wherein the lipid binding protein-based complex is CER-001.

37. The composition of claim 35 or claim 36, wherein the one or more ophthalmic drugs comprise a steroid, a kinase inhibitor, an angiotensin II receptor antagonist, an aldose reductase inhibitor, an immunosuppressant, a carbonic anhydrase inhibitor, an antimicrobial agent, an antiviral agent, an antihistamine, an anti-inflammatory, or a combination thereof.

38. The composition of any one of claims 35 to 37, wherein the one or more ophthalmic drugs comprise dexamethasone palmitate, azithromycin, dexamethasone, difluprednate, estradiol, fluocinolone, fluorometholone, hydrocortisone, loteprednol etabonate, prednisolone, triamcinolone, rimexolone, spironolactone, axitinib, BMS-794833 (N-(4-((2-amino-3-chloropyridin-4-yl)oxy)-3-fluorophenyl)-5-(4-fluorophenyl)-4-oxo-1,4-dihydropyridine-3-carboxamide), carbozantinib, cediranib, dovitinib, lapatinib, lenvatinib, motesanib, nintedanib, orantinib, PD173074 (N-[2-[[4-(Diethylamino)butyl]amino]-6-(3,5-dimethoxyphenyl) pyri-do[2,3-d]pyrimidin-7-yl]-N′-(1,1-dimethylethyl) urea), pazopanib, regorafenib, sorafenib, tofacitinib, ZM323881 (5-((7-Benzyloxyquinazolin-4-yl)amino)-4-fluoro-2-methylphenol), candesartan, irbesartan, losartan, olmesartan, telmisartan, valsartan, 2-methylsorbino, sirolimus, cyclosporine, tacrolimus, acetazolamide, brinzolamide, dorzolamide, ethoxzolamide, methazolamide, acyclovir, chloramphenicol, chlortetracycline, ciprofloxacin, fusidic acid, gancyclovir, norfloxacin, ofloxacin, tetracycline, zidovudine, levocabastine, bromfenac, diclofenac, indomethacin, nepafenac, or a combination thereof.

39. The composition of claim 38, wherein the one or more ophthalmic drugs comprise dexamethasone palmitate, optionally wherein the concentration of dexamethasone palmitate in the composition is 1 mg/ml.

40. The composition of claim 38, wherein the one or more ophthalmic drugs comprise dexamethasone.

41. The composition of claim 38, wherein the one or more ophthalmic drugs comprise ophthalmic drugs comprise tacrolimus.

42. The composition of any one of claims 35 to 41, which is a pharmaceutical composition further comprising one or more buffers, preservatives, excipients, diluents, or a combination thereof, optionally wherein the pharmaceutical composition is formulated as an eye drop.

43. A composition which is an implant comprising a lipid binding protein-based complex, optionally wherein the lipid binding protein-based complex is CER-001, optionally wherein the implant is a biodegradable or non-biodegradable disc, sheet, plug, rod, or pellet.

44. The composition of any one of claims 1 to 43 for use in a method of treating an eye disease in a subject.

45. A method of treating an eye disease in a subject, comprising:
(a) administering to the subject an Apolipoprotein A-I (“ApoA-I”) formulation comprising ApoA-I and one or more lipids, wherein the ApoA-I and the lipids are in the form of lipoprotein complexes, wherein:
(i) the subject has a cataract, uveitis, an eye disease associated with lipid accumulation, lipid keratopathy, corneal dystrophy, corneal opacity, dry eye disease, blepharitis, an inflammatory eye disease, macular edema (e.g., diabetic macular edema), macular degeneration, retinal detachment, an ocular tumor, a fungal infection, a viral infection, a bacterial infection (e.g., bacterial conjunctivitis or trachoma), multifocal choroiditis, diabetic retinopathy, proliferative vitreoretinopathy (PVR), sympathetic ophthalmia, Vogt Koyanagi-Harada (VKH) syndrome, histoplasmosis, uveal diffusion, vascular occlusion, endophthalmitis, glaucoma, Stargardt disease, ocular lipid deposits; and/or
(ii) the formulation is administered by suprachoroidal injection, suprascleral injection, an implant, iontophoresis, or electroporation.
